(19) United States
(12) Patent Application Publication
(10) Pub. No.: US 2024/0109943 A1
(43) Pub. Date: Apr. 4, 2024

(54) FISH ALLERGY ANTIGEN

(71) Applicant: HOYU CO., LTD., Aichi (JP)

(72) Inventors: Kayoko Matsunaga, Aichi (JP); Akiko Yagami, Aichi (JP); Naoshi Shimojo, Aichi (JP)

(73) Assignee: HOYU CO., LTD., Aichi (JP)

(21) Appl. No.: 18/491,287

(22) Filed: Oct. 20, 2023

Related U.S. Application Data
(62) Division of application No. 16/309,453, filed on Dec. 13, 2018.

(30) Foreign Application Priority Data
Jun. 16, 2016 (JP) 2016-119919

(51) Int. Cl. C07K 14/46; A23L 17/00; A23L 17/30; A23L 33/00; A61K 39/00; A61K 39/35; A61P 37/08; C12Q 1/48; C12Q 1/6888; G01N 33/53; G01N 33/68

(52) U.S. Cl. CPC C07K 14/461; A23L 17/00; A23L 17/30; A23L 33/40; A61K 39/00; A61K 39/35; A61P 37/08; C12Q 1/48; C12Q 1/6888; G01N 33/53; G01N 33/6854; G01N 33/6893; A23V 2002/00; C07K 14/47; G01N 2800/24

(57) ABSTRACT

The present invention provides novel antigens of an allergy to fish, methods and kits for diagnosing an allergy to fish, a pharmaceutical composition comprising the antigen, fish or fish eggs in which the antigen is eliminated, a processed product of the fish or the fish eggs or fish which lay the fish eggs or have hatched from the eggs, and a tester for determining the presence or absence of a fish antigen in an object of interest.

### CROSS-REFERENCE TO RELATED APPLICATIONS

This application is a divisional application of U.S. patent application Ser. No. 16/309,453 filed on Dec. 13, 2018, which is a U.S. national stage filing, under 35 U.S.C. § 371(c), of International Application No. PCT/JP2017/017018, filed on Apr. 28, 2017, which claims priority to Japanese Patent Application No. 2016-119919, filed on Jun. 16, 2016. The entire contents of each of the aforementioned applications, including drawings and sequence listings, are incorporated herein by reference.

### REFERENCE TO SEQUENCE LISTING

The instant application contains a Sequence Listing XML file which has been submitted electronically in XML format and is hereby incorporated by reference in its entirety. Said XML file, created on Oct. 19, 2023 is named Seq_Listing_129249_00403. XML and is 235,242 bytes in size.

### TECHNICAL FIELD

The present invention relates to a novel antigen of an allergy to fish. The present invention also relates to a kit, a composition, and a method for diagnosing allergy to fish. The present invention also relates to a pharmaceutical composition comprising the antigen and fish or fish eggs in which the antigen is eliminated or reduced, a processed product of the fish or the fish eggs or fish which lay the fish eggs or have hatched from the eggs. The present invention further relates to a tester for determining the presence or absence of a fish antigen in an object of interest.

### BACKGROUND ART

In blood and tissues of allergic patients, IgE antibodies specific to particular antigens are produced. Physiological consequences caused by interaction between such IgE antibodies and such particular antigens elicit allergic reactions.

In the process of production of conventional allergy testing agents, antigen reagents are commonly prepared simply by grinding a candidate allergenic food, food material or the like (Patent Literature 1). Therefore, numerous proteins were contained in a reagent and there the content of each protein was very little. For this reason, the only case where conventional allergy tests have permitted detection of a positive allergic reaction is when in a conventional antigen reagent containing many proteins, a particular antigen protein which causes the allergic reaction (allergen component) is present in an amount exceeding a threshold that allows determination of a positive reaction for binding to an IgE antibody. However, no determination of a positive reaction was possible and diagnosis efficiency was not sufficiently high when using a conventional allergy testing agent in patients possessing an IgE antibody binding to an allergen component present in small amounts in an allergen such as food, food material or the like.

The severity and symptoms of an allergic reaction do not necessarily correlate with the content of an allergen component. Even when a patient's IgE antibody reacts with an allergen component present in trace amounts in a candidate allergic food and food material, the allergic reaction may develop allergic symptoms or may affect the severity of those symptoms.

An attempt to increase the diagnostic efficiency is being made by examining IgE antibodies to each protein composing food and food material to distinguish sensitization that directly contributes to a diagnosis from sensitization based on cross-antigenicity by a pan-allergen or the like. The allergens set forth in Table below are currently known as fish allergens (Non Patent Literature 1).

TABLE 1

| | | | | | | |
|---|---|---|---|---|---|---|
| | | | | Food | | |
| Species | Allergen | Biochemical name | MW(SDS-PAGE) | Allergen | Entry Date | Modified Date |

Clupea harengus (Atlantic herring)

| | | | | | | |
|---|---|---|---|---|---|---|
| Clu h 1 | Beta-parvalbumin | 12 | kDa | Yes | 2016 Apr. 4 | 2014 Nov. 3 |

Cyprinus carpio (Common carp)

| | | | | | |
|---|---|---|---|---|---|
| Cyp c 1 | beta-parvalbumin | 12 | Yes | 2016 Apr. 4 | 2013 Sep. 10 |

Gadus callarias (Baltic cod)

| | | | | | |
|---|---|---|---|---|---|
| Gad c 1 | Beta-parvalbumin | 12 | Yes | 2016 Apr. 4 | 2010 Apr. 29 |

Gadus morhua (Atlantic cod)

| Gad m 1 | Beta-parvalbumin | 12 | | Yes | 2016 Apr. 4 | 2011 Jan. 25 |
|---|---|---|---|---|---|---|
| Gad m 2 | Beta-enolase | 47.3 | kDa | Yes | 2016 Apr. 4 | 2012 Jul. 30 |
| Gad m 3 | Aldolase A | 40 | kDa | Yes | 2016 Apr. 4 | 2012 Jul. 30 |

Lates calcarifer (Barramundi)

| | | | | | | |
|---|---|---|---|---|---|---|
| Lat c 1 | Beta-parvalbumin | 11.5 | kDa | Yes | 2016 Apr. 4 | 2014 Nov. 13 |

Lepidorhombus whiffiagonis (Megrim, Whiff, Gallo)

| | | | | | | |
|---|---|---|---|---|---|---|
| Lep w 1 | Beta-parvalbumin | 11.5 | kDa | Yes | 2016 Apr. 4 | 2010 Apr. 29 |

Oncorhynchus keta (Chum salmon)

| | | | | | | |
|---|---|---|---|---|---|---|
| Onc k 5 | beta-prime-component | 18 | kDa | Yes | 2016 Apr. 4 | 2012 Dec. 18 |
| | of vitellogenin | | | | | |

Oncorhynchus mykiss (Rainbow trout)

| | | | | | | |
|---|---|---|---|---|---|---|
| Onc m 1 | Beta-parvalbumin | 12 | kDa | Yes | 2016 Apr. 4 | 2011 Apr. 14 |

Oreochromis mossambicus (Mozambique tilapia)

| | | | | | | |
|---|---|---|---|---|---|---|
| Ore m 4 | Tropomyosin | 33 | kDa | Yes | 2016 Apr. 4 | 2012 Jul. 30 |

Salmo salar (Atlantic salmon)

| Sal s 1 | Beta-parvalbumin 1 | 12 | | Yes | 2016 Apr. 4 | 2010 Apr. 29 |
|---|---|---|---|---|---|---|
| Sal s 2 | Beta-Enolase | 47.3 | kDa | Yes | 2016 Apr. 4 | 2011 Aug. 26 |

| | | | | | |
|---|---|---|---|---|---|
| Sal s 3 | Aldolase A | 40 | Yes | 2016 Apr. 4 | 2012 Jul. 30 |

Sardinops sagax (Pacific pilchard)

| | | | | | | |
|---|---|---|---|---|---|---|
| Sar sa 1 | Beta-parvalbumin | 12 | kDa | Yes | 2016 Apr. 4 | 2010 Apr. 29 |

Sebastes marinus (Ocean perch, redfish, snapper)

| | | | | | | |
|---|---|---|---|---|---|---|
| Seb m 1 | Beta-parvalbumin | 11 | kDa | Yes | 2016 Apr. 4 | 2010 Apr. 29 |

Thunnus albacares (Yellowfin tuna)

| | | | | | | |
|---|---|---|---|---|---|---|
| Thu a 1 | Beta-parvalbumin | 11 | kDa | Yes | 2016 Apr. 4 | 2010 Apr. 29 |

| | | | | | |
|---|---|---|---|---|---|
| Thu a 2 | Beta-enolase | 50 | Yes | 2016 Apr. 4 | 2012 Jul. 30 |
| Thu a 3 | Aldolase A | 40 | Yes | 2016 Apr. 4 | 2012 Jul. 30 |

Xiphias gladius (Swordfish)

| | | | | | | |
|---|---|---|---|---|---|---|
| Xip g 1 | Beta-parvalbumin | 11.5 | kDa | Yes | 2016 Apr. 4 | 2010 Apr. 29 |

However, while it is necessary to exhaustively identify allergen components in candidate allergic foods and food materials in order to enhance the reliability of allergy tests, the patient detection rate by the measurement of such allergen components is far from sufficient. Identification of novel allergens in fish is very important not only for increasing the precision of diagnosis, but also for determining targets of therapeutic agents and low allergenic foods and low allergenic food materials.

Meanwhile, in the field of protein separation and purification, various efforts have conventionally been made to develop methods for separating and purifying a protein or nucleic acid of interest from cell extracts or the like. Such methods may well be exemplified by dialysis based on salt concentration, and centrifugal separation.

Other efforts have been made to develop many purification methods based on electric charges of protein or nucleic acid residues or on the difference in molecular weight. Electric charge-based purification methods can be exemplified by column chromatography using ion exchange resins, and isoelectric focusing. Purifications based on molecular weight difference can be exemplified by centrifugal separation, molecular-sieve column chromatography, and SDS-PAGE (sodium dodecyl sulfate-polyacrylamide gel electrophoresis).

In recent years, a method for separating and purifying many different proteins from a small amount of sample has been used, which is more specifically a two-dimensional electrophoresis consisting of isoelectric focusing in the first dimension, followed by SDS-PAGE in the second dimension. The present applicant has conventionally developed some 2D electrophoresis methods with high separation ability (Patent Literature 2-5).

### CITATION LIST

### Patent Literature

PTL1: Japanese Patent Application Publication No. JP 2002-286716
PTL2: Japanese Patent Application Publication No. JP 2011-33544
PTL3: Japanese Patent Application Publication No. JP 2011-33546
PTL4: Japanese Patent Application Publication No. JP 2011-33547
PTL5: Japanese Patent Application Publication No. JP 2011-33548

### Non-Patent Literature

NPTL1: Allergen Nomenclature, WHO/IUIS Allergen Nomenclature Sub-Committee, [searched on Jun. 9, 2016], internet<URL: http://www.allergen.org/search.php?allergenname=&allergensource=&TaxSource=Animalia+Chordata&TaxOrder=&foodallerg=1&bioname=>

### SUMMARY OF INVENTION

### Technical Problem

The present invention provides novel antigens of an allergy to fish. The present invention also provides methods and kits for diagnosing allergy to fish. The present invention also provides pharmaceutical compositions comprising such an antigen and fish or fish eggs in which such an antigen is eliminated or reduced, a processed product of the fish or the fish eggs or fish which lay the fish eggs or have hatched from the eggs.

### Solution to Problem

In order to solve the aforementioned problems, the present inventors had made intensive studies to identify causative antigens of an allergy to fish. As a result, the inventors succeeded in identifying novel antigens to which an IgE antibody in the serum of a fish-allergic patient specifically binds. The present invention has been completed based on this finding.

Thus, in one embodiment, the present invention can be as defined below.

[1] A kit for diagnosing a fish allergy, the kit comprising, as an antigen, at least one protein defined in any one of the following (1) to (9):
(1) (1A) Alpha-actinin-3 or a variant thereof, which is a protein defined below in any of (1A-a) to (1A-e) which is an antigen for the fish allergy:
   (1A-a) a protein comprising an amino acid sequence with deletion, substitution, insertion or addition of one or several amino acids in SEQ ID NO: 2;
   (1A-b) a protein comprising an amino acid sequence having at least 70% identity to the amino acid sequence of SEQ ID NO: 2;
   (1A-c) a protein comprising an amino acid sequence encoded by a nucleotide sequence with deletion, substitution, insertion or addition of one or several nucleotides in SEQ ID NO: 1;
   (1A-d) a protein comprising an amino acid sequence encoded by a nucleotide sequence having at least 70% identity to the nucleotide sequence of SEQ ID NO: 1; or
   (1A-e) a protein comprising an amino acid sequence encoded by a nucleic acid that hybridizes under stringent conditions with a nucleic acid having a nucleotide sequence complementary to the nucleotide sequence of SEQ ID NO: 1; or
   (1B) a protein comprising an amino acid sequence of SEQ ID NO: 2 or 3;
(2) (2A) EEF1A2 binding protein-like or a variant thereof, which is a protein defined below in any of (2A-a) to (2A-e) which is an antigen for the fish allergy:
   (2A-a) a protein comprising an amino acid sequence with deletion, substitution, insertion or addition of one or several amino acids in SEQ ID NO: 5;
   (2A-b) a protein comprising an amino acid sequence having at least 70% identity to the amino acid sequence of SEQ ID NO: 5;
   (2A-c) a protein comprising an amino acid sequence encoded by a nucleotide sequence with deletion, substitution, insertion or addition of one or several nucleotides in SEQ ID NO: 4;
   (2A-d) a protein comprising an amino acid sequence encoded by a nucleotide sequence having at least 70% identity to the nucleotide sequence of SEQ ID NO: 4; or
   (2A-e) a protein comprising an amino acid sequence encoded by a nucleic acid that hybridizes under stringent conditions with a nucleic acid having a nucleotide sequence complementary to the nucleotide sequence of SEQ ID NO: 4; or
   (2B) a protein comprising at least one amino acid sequence selected from the group consisting of SEQ ID NOs: 5-8;
(3) (3A) Alpha-1,4-glucan phosphorylase or a variant thereof, which is a protein defined below in any of (3A-a) to (3A-e) which is an antigen for the fish allergy:
   (3A-a) a protein comprising an amino acid sequence with deletion, substitution, insertion or addition of one or several amino acids in SEQ ID NO: 10;
   (3A-b) a protein comprising an amino acid sequence having at least 70% identity to the amino acid sequence of SEQ ID NO: 10;
   (3A-c) a protein comprising an amino acid sequence encoded by a nucleotide sequence with deletion, substitution, insertion or addition of one or several nucleotides in SEQ ID NO: 9;
   (3A-d) a protein comprising an amino acid sequence encoded by a nucleotide sequence having at least 70% identity to the nucleotide sequence of SEQ ID NO: 9; or
   (3A-e) a protein comprising an amino acid sequence encoded by a nucleic acid that hybridizes under stringent conditions with a nucleic acid having a nucleotide sequence complementary to the nucleotide sequence of SEQ ID NO: 9; or
   (3B) a protein comprising at least one amino acid sequence selected from the group consisting of SEQ ID NOs: 10-16;
(4) (4A) Elongation factor 2 or a variant thereof, which is a protein defined below in any of (4A-a) to (4A-e) which is an antigen for the fish allergy:
   (4A-a) a protein comprising an amino acid sequence with deletion, substitution, insertion or addition of one or several amino acids in SEQ ID NO: 19;
   (4A-b) a protein comprising an amino acid sequence having at least 70% identity to the amino acid sequence of SEQ ID NO: 19;
   (4A-c) a protein comprising an amino acid sequence encoded by a nucleotide sequence with deletion, substitution, insertion or addition of one or several nucleotides in SEQ ID NO: 18;
   (4A-d) a protein comprising an amino acid sequence encoded by a nucleotide sequence having at least 70% identity to the nucleotide sequence of SEQ ID NO: 18; or
   (4A-e) a protein comprising an amino acid sequence encoded by a nucleic acid that hybridizes under stringent conditions with a nucleic acid having a nucleotide sequence complementary to the nucleotide sequence of SEQ ID NO: 18; or
   (4B) a protein comprising at least one amino acid sequence selected from the group consisting of SEQ ID NOs: 19-24;
(5) (5A) Heat shock cognate 70 kDa protein or a variant thereof, which is a protein defined below in any of (5A-a) to (5A-e) which is an antigen for the fish allergy:
   (5A-a) a protein comprising an amino acid sequence with deletion, substitution, insertion or addition of one or several amino acids in SEQ ID NO: 26;
   (5A-b) a protein comprising an amino acid sequence having at least 70% identity to the amino acid sequence of SEQ ID NO: 26;
   (5A-c) a protein comprising an amino acid sequence encoded by a nucleotide sequence with deletion, substitution, insertion or addition of one or several nucleotides in SEQ ID NO: 25;
   (5A-d) a protein comprising an amino acid sequence encoded by a nucleotide sequence having at least 70% identity to the nucleotide sequence of SEQ ID NO: 25; or
   (5A-e) a protein comprising an amino acid sequence encoded by a nucleic acid that hybridizes under stringent conditions with a nucleic acid having a nucleotide sequence complementary to the nucleotide sequence of SEQ ID NO: 25; or
   (5B) a protein comprising at least one amino acid sequence selected from the group consisting of SEQ ID NOs: 26-31;
(6) (6A) Serotransferrin or a variant thereof, which is a protein defined below in any of (6A-a) to (6A-e) which is an antigen for the fish allergy:
   (6A-a) a protein comprising an amino acid sequence with deletion, substitution, insertion or addition of one or several amino acids in SEQ ID NO: 33;
   (6A-b) a protein comprising an amino acid sequence having at least 70% identity to the amino acid sequence of SEQ ID NO: 33;
   (6A-c) a protein comprising an amino acid sequence encoded by a nucleotide sequence with deletion, substitution, insertion or addition of one or several nucleotides in SEQ ID NO: 32;
   (6A-d) a protein comprising an amino acid sequence encoded by a nucleotide sequence having at least 70% identity to the nucleotide sequence of SEQ ID NO: 32; or
   (6A-e) a protein comprising an amino acid sequence encoded by a nucleic acid that hybridizes under stringent conditions with a nucleic acid having a nucleotide sequence complementary to the nucleotide sequence of SEQ ID NO: 32; or
   (6B) a protein comprising at least one amino acid sequence selected from the group consisting of SEQ ID NOs: 33-41;
(7) (7A) Myosin binding protein H-like or a variant thereof, which is a protein defined below in any of (7A-a) to (7A-e) which is an antigen for the fish allergy:
   (7A-a) a protein comprising an amino acid sequence with deletion, substitution, insertion or addition of one or several amino acids in SEQ ID NO: 43;
   (7A-b) a protein comprising an amino acid sequence having at least 70% identity to the amino acid sequence of SEQ ID NO: 43;
   (7A-c) a protein comprising an amino acid sequence encoded by a nucleotide sequence with deletion, substitution, insertion or addition of one or several nucleotides in SEQ ID NO: 42;
   (7A-d) a protein comprising an amino acid sequence encoded by a nucleotide sequence having at least 70% identity to the nucleotide sequence of SEQ ID NO: 42; or
   (7A-e) a protein comprising an amino acid sequence encoded by a nucleic acid that hybridizes under stringent conditions with a nucleic acid having a nucleotide sequence complementary to the nucleotide sequence of SEQ ID NO: 42; or
   (7B) a protein comprising at least one amino acid sequence selected from the group consisting of SEQ ID NOs: 43-54,
(8) (8A) Desmin (Fragment) or a variant thereof, which is a protein defined below in any of (8A-a) to (8A-e) which is an antigen for the fish allergy:
   (8A-a) a protein comprising an amino acid sequence with deletion, substitution, insertion or addition of one or several amino acids in SEQ ID NO: 56;
   (8A-b) a protein comprising an amino acid sequence having at least 70% identity to the amino acid sequence of SEQ ID NO: 56;
   (8A-c) a protein comprising an amino acid sequence encoded by a nucleotide sequence with deletion, substitution, insertion or addition of one or several nucleotides in SEQ ID NO: 55;
   (8A-d) a protein comprising an amino acid sequence encoded by a nucleotide sequence having at least 70% identity to the nucleotide sequence of SEQ ID NO: 55; or
   (8A-e) a protein comprising an amino acid sequence encoded by a nucleic acid that hybridizes under stringent conditions with a nucleic acid having a nucleotide sequence complementary to the nucleotide sequence of SEQ ID NO: 55; or
   (8B) a protein comprising at least one amino acid sequence selected from the group consisting of SEQ ID NOs: 56-59;
(9) (9A) Capping protein (Actin filament) muscle Z-line beta or a variant thereof, which is a protein defined below in any of (9A-a) to (9A-e) which is an antigen for the fish allergy:
   (9A-a) a protein comprising an amino acid sequence with deletion, substitution, insertion or addition of one or several amino acids in SEQ ID NO: 61;
   (9A-b) a protein comprising an amino acid sequence having at least 70% identity to the amino acid sequence of SEQ ID NO: 61;
   (9A-c) a protein comprising an amino acid sequence encoded by a nucleotide sequence with deletion, substitution, insertion or addition of one or several nucleotides in SEQ ID NO: 60;
   (9A-d) a protein comprising an amino acid sequence encoded by a nucleotide sequence having at least 70% identity to the nucleotide sequence of SEQ ID NO: 60; or (9A-e) a protein comprising an amino acid sequence encoded by a nucleic acid that hybridizes under stringent conditions with a nucleic acid having a nucleotide sequence complementary to the nucleotide sequence of SEQ ID NO: 60; or
   (9B) a protein comprising at least one amino acid sequence selected from the group consisting of SEQ ID NOs: 61-68.

[2] A composition for diagnosing a fish allergy, the composition comprising, as an antigen, at least one of proteins as defined above in any of (1) to (9) of [1].

[3] A method for providing an indicator for diagnosing a fish allergy in a subject, the method comprising the steps of:
(i) contacting a sample obtained from the subject with an antigen, wherein the sample is a solution comprising an IgE antibody;
(ii) detecting binding between an IgE antibody present in the sample from the subject and the antigen; and
(iii) when the binding between the IgE antibody in the subject and the antigen is detected, an indicator of the fact that the subject has a fish allergy is provided;
   wherein the antigen is at least one of proteins as defined above in any of (1) to (9) of [1].

[4] A pharmaceutical composition comprising at least one of proteins as defined above in any of (1) to (9) of [1].

[5] The pharmaceutical composition as set forth in [4] for the treatment of a fish allergy.

[6] A fish or a processed product of fish, in which an antigen is eliminated or reduced, wherein the antigen is at least one of proteins as defined above in any of (1) to (9) of [1].

[7] A tester for determining the presence or absence of a fish antigen in an object of interest, the tester comprising an antibody that binds to at least one of proteins as defined above in any of (1) to (9) of [1].

[8] A tester for determining the presence or absence of an antigen causing an allergy to fish in an object of interest, the tester comprising a primer having a nucleotide sequence complementary to at least one part of one of the nucleotide sequences set forth in SEQ ID NO: 1, 4, 9, 18, 25, 32, 42, 55, or 60.

[9] A kit for diagnosing a fish allergy, the kit comprising, as an antigen, at least one protein defined in any one of the following (10) to (14):
(10) (10A) Myosin heavy chain, fast skeletal muscle-like or a variant thereof, which is a protein defined below in any of (10A-a) to (10A-e) which is an antigen for the fish allergy:
   (10A-a) a protein comprising an amino acid sequence with deletion, substitution, insertion or addition of one or several amino acids in SEQ ID NO: 70;
   (10A-b) a protein comprising an amino acid sequence having at least 70% identity to the amino acid sequence of SEQ ID NO: 70;
   (10A-c) a protein comprising an amino acid sequence encoded by a nucleotide sequence with deletion, substitution, insertion or addition of one or several nucleotides in SEQ ID NO: 69;
   (10A-d) a protein comprising an amino acid sequence encoded by a nucleotide sequence having at least 70% identity to the nucleotide sequence of SEQ ID NO: 69; or
   (10A-e) a protein comprising an amino acid sequence encoded by a nucleic acid that hybridizes under stringent conditions with a nucleic acid having a nucleotide sequence complementary to the nucleotide sequence of SEQ ID NO: 69; or
   (10B) a protein comprising at least one amino acid sequence selected from the group consisting of SEQ ID NOs: 70-108;
(11) (11A) Glycogen phosphorylase, muscle form-like or a variant thereof, which is a protein defined below in any of (11A-a) to (11A-e) which is an antigen for the fish allergy:
   (11A-a) a protein comprising an amino acid sequence with deletion, substitution, insertion or addition of one or several amino acids in SEQ ID NO: 110;
   (11A-b) a protein comprising an amino acid sequence having at least 70% identity to the amino acid sequence of SEQ ID NO: 110;
   (11A-c) a protein comprising an amino acid sequence encoded by a nucleotide sequence with deletion, substitution, insertion or addition of one or several nucleotides in SEQ ID NO: 109;
   (11A-d) a protein comprising an amino acid sequence encoded by a nucleotide sequence having at least 70% identity to the nucleotide sequence of SEQ ID NO: 109; or
   (11A-e) a protein comprising an amino acid sequence encoded by a nucleic acid that hybridizes under stringent conditions with a nucleic acid having a nucleotide sequence complementary to the nucleotide sequence of SEQ ID NO: 109; or
   (11B) a protein comprising at least one amino acid sequence selected from the group consisting of SEQ ID NOs: 110-119;
(12) (12A) Myosin-binding protein C, fast-type-like or a variant thereof, which is a protein defined below in any of (12A-a) to (12A-e) which is an antigen for the fish allergy:
   (12A-a) a protein comprising an amino acid sequence with deletion, substitution, insertion or addition of one or several amino acids in SEQ ID NO: 121;
   (12A-b) a protein comprising an amino acid sequence having at least 70% identity to the amino acid sequence of SEQ ID NO: 121;
   (12A-c) a protein comprising an amino acid sequence encoded by a nucleotide sequence with deletion, substitution, insertion or addition of one or several nucleotides in SEQ ID NO: 120;
   (12A-d) a protein comprising an amino acid sequence encoded by a nucleotide sequence having at least 70% identity to the nucleotide sequence of SEQ ID NO: 120; or
   (12A-e) a protein comprising an amino acid sequence encoded by a nucleic acid that hybridizes under stringent conditions with a nucleic acid having a nucleotide sequence complementary to the nucleotide sequence of SEQ ID NO: 120; or
   (12B) a protein comprising at least one amino acid sequence selected from the group consisting of SEQ ID NOs: 121-136;
(13) (13A) ATP synthase subunit beta, mitochondrial or a variant thereof, which is a protein defined below in any of (13A-a) to (13A-e) which is an antigen for the fish allergy:
   (13A-a) a protein comprising an amino acid sequence with deletion, substitution, insertion or addition of one or several amino acids in SEQ ID NO: 138;
   (13A-b) a protein comprising an amino acid sequence having at least 70% identity to the amino acid sequence of SEQ ID NO: 138;
   (13A-c) a protein comprising an amino acid sequence encoded by a nucleotide sequence with deletion, substitution, insertion or addition of one or several nucleotides in SEQ ID NO: 137;
   (13A-d) a protein comprising an amino acid sequence encoded by a nucleotide sequence having at least 70% identity to the nucleotide sequence of SEQ ID NO: 137; or
   (13A-e) a protein comprising an amino acid sequence encoded by a nucleic acid that hybridizes under stringent conditions with a nucleic acid having a nucleotide sequence complementary to the nucleotide sequence of SEQ ID NO: 137; or
   (13B) a protein comprising at least one amino acid sequence selected from the group consisting of SEQ ID NOs: 138-142;
(14) (14A) L-lactate dehydrogenase A chain-like or a variant thereof, which is a protein defined below in any of (14A-a) to (14A-e) which is an antigen for the fish allergy:
   (14A-a) a protein comprising an amino acid sequence with deletion, substitution, insertion or addition of one or several amino acids in SEQ ID NO: 144;
   (14A-b) a protein comprising an amino acid sequence having at least 70% identity to the amino acid sequence of SEQ ID NO: 144;
   (14A-c) a protein comprising an amino acid sequence encoded by a nucleotide sequence with deletion, substitution, insertion or addition of one or several nucleotides in SEQ ID NO: 143;
   (14A-d) a protein comprising an amino acid sequence encoded by a nucleotide sequence having at least 70% identity to the nucleotide sequence of SEQ ID NO: 143; or
   (14A-e) a protein comprising an amino acid sequence encoded by a nucleic acid that hybridizes under stringent conditions with a nucleic acid having a nucleotide sequence complementary to the nucleotide sequence of SEQ ID NO: 143; or
   (14B) a protein comprising at least one amino acid sequence selected from the group consisting of SEQ ID NOs: 144-149.

[10] A composition for diagnosing a fish allergy, the composition comprising, as an antigen, at least one of proteins as defined above in any of (10) to (14) of [9].

[11] A method for providing an indicator for diagnosing a fish allergy in a subject, the method comprising the steps of:
(i) contacting a sample obtained from the subject with an antigen, wherein the sample is a solution comprising an IgE antibody;
(ii) detecting binding between an IgE antibody present in the sample from the subject and the antigen; and
(iii) when the binding between the IgE antibody in the subject and the antigen is detected, an indicator of the fact that the subject has a fish allergy is provided;
   wherein the antigen is at least one of proteins as defined above in any of (10) to (14) of [9].

[12] A pharmaceutical composition comprising at least one of proteins as defined above in any of (10) to (14) of [9].

[13] The pharmaceutical composition as set forth in for the treatment of a fish allergy.

[14] A fish or a processed product of fish, in which an antigen is eliminated or reduced, wherein the antigen is at least one of proteins as defined above in any of (10) to (14) of [9].

[15] A tester for determining the presence or absence of a fish antigen in an object of interest, the tester comprising an antibody that binds to at least one of proteins as defined above in any of (10) to (14) of [9].

[16] A tester for determining the presence or absence of an antigen causing an allergy to fish in an object of interest, the tester comprising a primer having a nucleotide sequence complementary to at least one part of one of the nucleotide sequences set forth in SEQ ID NO: 69, 109, 120, 137, or 143.

[17] A fish-derived antigen which is at least one of proteins as defined above in any of (1) to (9) of [1] and causes an allergy to fish.

[18] A fish-derived antigen which is at least one of proteins as defined above in any of (10) to (14) of [9] and causes an allergy to fish.

### Advantageous Effects of Invention

The present invention can provide novel antigens of an allergy to fish. Since the novel antigens (allergen components) that trigger a fish allergy were identified in the present invention, it is possible to provide highly sensitive methods and kits for diagnosing an allergy to fish, pharmaceutical compositions comprising such an antigen, fish or fish eggs in which the antigen is eliminated or reduced, a processed product of the fish or the fish eggs or fish which lay the fish eggs or have hatched from the eggs, and testers for determining the presence or absence of a fish antigen in an object of interest.

### BRIEF DESCRIPTION OF DRAWINGS

FIG. 1A is a photograph of a gel showing a protein electrophoretic pattern in two-dimensional electrophoresis of proteins contained in an extract of flesh of salmon (left panel) and a photograph of an immunoblot obtained from the two-dimensional electrophoretic pattern by using a serum of Fish-allergic patient 1 (right panel). The bands at the left of the photograph of the gel are bands of molecular weight markers and the numbers on the left side of the photograph of the gel are molecular weights of respective molecular weight markers (KDa). The numbers on the upper side of the photograph indicate isoelectric points.

FIG. 1B is a photograph of an immunoblot obtained from the two-dimensional electrophoretic pattern of the proteins contained in the extract of the flesh of salmon by using a serum of Fish-allergic patient 2 (left panel) and a photograph of an immunoblot of the proteins stained with serum of Fish-allergic patient 3 (right panel). The numbers on the upper side of each photograph indicate isoelectric points.

FIG. 2 is photographs of immunoblots obtained from two-dimensional electrophoretic patterns of proteins contained in extracts of the flesh of 10 species of fish stained with serum of Fish-allergic patient 1.

FIG. 3 is photographs of immunoblots obtained from two-dimensional electrophoretic patterns of proteins contained in extracts of the flesh of 10 species of fish stained with serum of a subject who does not exhibit fish allergic symptoms (Non-fish-allergic subject).

FIG. 4 is photographs of immunoblots obtained from two-dimensional electrophoretic patterns of proteins contained in extracts of the fish of 6 species of fish by using a serum of Fish-allergic patient 4.

### DESCRIPTION OF EMBODIMENTS

The present invention will be described in detail below, but the present invention is not limited to them.

Unless otherwise defined herein, all scientific and technical terms used in relation to the present invention shall have meanings commonly understood by those skilled in the art.

As referred to herein, the “allergy” refers to the state in which, when a certain antigen reenters a living organism sensitized to said antigen, the living organism shows a hypersensitive reaction detrimental to the living organism. In blood and tissues of individuals with many food-allergic diseases, IgE antibodies specific to antigens are produced. IgE antibodies bind to mast cells or basophils. When an antigen specific to such an IgE antibody reenters the body of a patient with an allergic disease, said antigen combines with the IgE antibody bound to mast cells or basophils, and the IgE antibody crosslinks said antigen on the cell surface, resulting in physiological effects of IgE antibody-antigen interaction. Examples of such physiological effects include release of histamine, serotonin, heparin, eosinophil chemotactic factors, leucotrienes, or the like. These released substances provoke an allergic reaction resulting from the combination of an IgE antibody with particular antigens. Such allergic reactions caused by particular antigens occur through the aforementioned pathway.

As used herein, the term fish means teleost and elasmobranch among fishes, preferably teleost, more preferably those belonging to Salmoniformes, Perciformes, Anguilliformes, Gadiformes, and Pleuronectiformes, further preferably those belonging to Salmonidae, Carangidae, Congridae, Sparidae, Scombridae, Gadidae, Anguillidae, and Paralichthyidae, and even preferably salmon, horse mackerel, sea eel, black porgy, mackerel, sea bream, cod, young yellowtail, eel, and flatfish. The fish may be edible.

As used herein, the term fish eggs means eggs of fish. The fish eggs may be edible.

As referred to herein, the “allergy to fish” refers to the state in which an individual has an allergic reaction caused by proteins, etc. present in fish which act as an antigen. The allergy to fish can produce an allergic reaction upon contact with, or consumption of, an antigen present in fish. In general, allergic reactions caused by consumption of foods are particularly referred to as “food allergies”. The allergy to fish may be a food allergy.

As referred to herein, the “antigen” refers to a substance that provokes an allergic reaction, and is also referred to as an “allergen component”. The antigen is preferably a protein.

As referred to herein, the “protein” refers to a molecule having a structure in which naturally occurring amino acids are joined together by peptide bond. The number of amino acids present in a protein is not particularly limited, but proteins having about 2 to 50 amino acids joined together by peptide bond are in some cases called “peptides”. In the case where amino acids can form different enantiomers, the amino acids are understood to form an L-enantiomer, unless otherwise indicated. The amino acid sequences of proteins or peptides as used herein are represented by one-letter symbols of amino acids in accordance with standard usage and the notational convention commonly used in the art. The leftward direction represents the amino-terminal direction, and the rightward direction represents the carboxy-terminal direction.

Identification of Antigens

Proteins contained in fish were analyzed by the aforementioned technique to identify antigens of an allergy to fish. To be specific, proteins were extracted from the flesh of fish and subjected to two-dimensional electrophoresis under the conditions described below.

The electrophoresis in the first dimension was isoelectric focusing, which was performed using isoelectric focusing gels with a gel-strip length of 5 to 10 cm and a gel pH range of 3 to 10. The pH gradient of the gels in the direction of electrophoresis was as follows: when the total gel-strip length is taken as 1, the gel-strip length up to pH 5 is taken as “a”, the gel-strip length from pH 5 to 7 is taken as “b”, and the gel-strip length above pH 7 is taken as “c”, “a” is in the range of 0.15 to 0.3, “b” is in the range of 0.4 to 0.7, and “c” is in the range of 0.15 to 0.3. More specifically, the isoelectric focusing was performed using the IPG gels, Immobiline Drystrip (pH3-10NL), produced by GE Healthcare Bio-Sciences Corporation (hereinafter abbreviated as “GE”). The electrophoresis system used was IPGphor produced by GE. The maximum current of the electrophoresis system was limited to 75 μA per gel strip. The voltage program adopted to perform the first-dimensional isoelectric focusing was as follows: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

The electrophoresis in the second dimension was SDS-PAGE, which was performed using polyacrylamide gels whose gel concentration at the distal end in the direction of electrophoresis was set to 3 to 6% and whose gel concentration at the proximal end was set to a higher value than that at the distal end. More specifically, the SDS-PAGE was performed using NuPAGE 4-12% Bris-Tris Gels (IPG well, Mini, 1 mm) produced by Life Technologies. The electrophoresis system used was XCell SureLock Mini-Cell produced by Life Technologies. The electrophoresis was run at a constant voltage of 200 V for about 45 minutes using an electrophoresis buffer composed of 50 mM MOPS, 50 mM Tris base, 0.1% (w/v) SDS and 1 mM EDTA.

As a result, the following spots in a two-dimensional electrophoresis gel run under the conditions described above for fish proteins have been revealed to exhibit specific binding to IgE antibodies from patients having an allergy to fish and being diagnosed as having immediate-type allergy.

Spot 1: Molecular weight 80 to 160 kDa, pI 3.0 to 7.0
Spot 2: Molecular weight 110 to 260 kDa, pI 4.0 to 8.0
Spot 3: Molecular weight 80 to 160 kDa, pI 4.0 to 10.0
Spot 4: Molecular weight 80 to 160 kDa, pI 5.0 to 11.0
Spot 5: Molecular weight 50 to 110 kDa, pI 3.0 to 7.0
Spot 6: Molecular weight 60 to 110 kDa, pI 4.0 to 8.0
Spot 7: Molecular weight 40 to 80 kDa, pI 4.0 to 8.0
Spot 8: Molecular weight 40 to 80 kDa, pI 3.0 to 7.0
Spot 9: Molecular weight 20 to 50 kDa, pI 3.0 to 7.0
Spot 10: Molecular weight 160 to 300 kDa, pI 3.0 to 7.0
Spot 11: Molecular weight 80 to 160 kDa, pI 4.0 to 8.0
Spot 12: Molecular weight 100 to 160 kDa, pI 4.0 to 7.0
Spot 13: Molecular weight 30 to 70 kDa, pI 3.0 to 7.0
Spot 14: Molecular weight 20 to 50 kDa, pI 5.0 to 9.0

Antigen

(1) Antigen in Spot 1

As the result of sequence identification of the antigen in spot 1 by mass spectroscopy, the following amino acid sequences of SEQ ID NO: 3 were detected.

Also, the mass spectroscopic data (SEQ ID NO: 3) obtained for spot 1 on a mass spectrometer was analyzed by comparing the data against the NCB1 protein data, and as a result, the antigen in question was identified as Alpha-actinin-3 (amino acid sequence: SEQ ID NO: 2, encoding nucleotide sequence: SEQ ID NO: 1). SEQ ID NO: 3 corresponds to amino acid 572 to 585 in SEQ ID NO: 2.

Accordingly, in the present invention, the antigen in spot 1 can be any of (1A-a) to (1A-e) and (1B) as defined below:
(1A-a) a protein comprising an amino acid sequence with deletion, substitution, insertion or addition of one or several amino acids in SEQ ID NO: 2;
(1A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 2;
(1A-c) a protein comprising an amino acid sequence encoded by a nucleotide sequence with deletion, substitution, insertion or addition of one or several nucleotides in SEQ ID NO: 1;
(1A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 1;
(1A-e) a protein comprising an amino acid sequence encoded by a nucleic acid that hybridizes under stringent conditions with a nucleic acid having a nucleotide sequence complementary to the nucleotide sequence of SEQ ID NO: 1;
(1B) a protein comprising at least one amino acid sequence of SEQ ID NO: 2 or 3, preferably a protein comprising all sequences of the amino acid sequences. As referred to above, the amino acid sequence of SEQ ID NO: 2 or 3 may be an amino acid sequence derived therefrom by deletion, substitution, insertion or addition of one or several amino acids.

The proteins of (1A-a) to (1A-e) and (1B) as defined above also include those proteins whose amino acid residues are modified by phosphorylation, sugar chain modification, aminoacylation, ring-opening, deamination or the like.

The proteins of (1A-a) to (1A-e) and (1B) as defined above can be proteins that are found in a protein spot with a molecular weight of around 80 to 160 kDa, preferably around 90 to 120 kDa and an isoelectric point of 3.0 to 7.0, preferably an isoelectric point of 3.0 to 6.0 on gels used in the two-dimensional electrophoresis performed under the conditions described above in the subsection titled “Identification of antigens”.

Preferably, a protein that is an antigen in spot 1 is an antigen of an allergy to fish.

(2) Antigen in Spot 2

As the result of sequence identification of the antigen in spot 2 by mass spectroscopy, the following amino acid sequences of SEQ ID NOs: 6-8 were detected.

Also, the mass spectroscopic data obtained for spot 2 (SEQ ID NOs: 6-8) on a mass spectrometer was analyzed by comparing the data against the UniProt protein data, and as a result, the antigen in question was identified as EEF1A2 binding protein-like (amino acid sequence: SEQ ID NO: 5, encoding nucleotide sequence: SEQ ID NO: 4). SEQ ID NO: 6 corresponds to amino acid 143-159 in SEQ ID NO: 5, SEQ ID NO: 7 corresponds to amino acid 395-404 in SEQ ID NO: 5, and SEQ ID NO: 8 corresponds to amino acid 427-447 in SEQ ID NO: 5.

Accordingly, in the present invention, the antigen in spot 2 can be any of (2A-a) to (2A-e) and (2B) as defined below:
(2A-a) a protein comprising an amino acid sequence with deletion, substitution, insertion or addition of one or several amino acids in SEQ ID NO: 5;
(2A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 5;
(2A-c) a protein comprising an amino acid sequence encoded by a nucleotide sequence with deletion, substitution, insertion or addition of one or several nucleotides in SEQ ID NO: 4;
(2A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 4;
(2A-e) a protein comprising an amino acid sequence encoded by a nucleic acid that hybridizes under stringent conditions with a nucleic acid having a nucleotide sequence complementary to the nucleotide sequence of SEQ ID NO: 4;
(2B) a protein comprising at least one amino acid sequence selected from the group consisting of SEQ ID NOs: 5-8, preferably a protein comprising at least 2, 3, or all sequences of the amino acid sequences. As referred to above, the amino acid sequence of any of SEQ ID NOs: 5-8 may be an amino acid sequence derived therefrom by deletion, substitution, insertion or addition of one or several amino acids.

The proteins of (2A-a) to (2A-e) and (2B) as defined above also include those proteins whose amino acid residues are modified by phosphorylation, sugar chain modification, aminoacylation, ring-opening, deamination or the like.

The proteins of (2A-a) to (2A-e) and (2B) as defined above can be proteins that are found in a protein spot with a molecular weight of around 110 to 260 kDa, preferably around 120 to 160 kDa and an isoelectric point of 4.0 to 8.0, preferably an isoelectric point of 4.0 to 7.0 on gels used in the two-dimensional electrophoresis performed under the conditions described above in the subsection titled “Identification of antigens”.

Preferably, a protein that is an antigen in spot 2 is an antigen of an allergy to fish.

(3) Antigen in Spot 3

As the result of sequence identification of the antigen in spot 3 by mass spectroscopy, the following amino acid sequences of SEQ ID NOs: 11-16 were detected.

Also, the mass spectroscopic data (SEQ ID NOs: 11-17) obtained for spot 3 on a mass spectrometer was analyzed by comparing the data against the Uniprot protein data, and as a result, the antigen in question was identified as Alpha-1, 4-glucan phosphorylase (amino acid sequence: SEQ ID NO: 10, encoding nucleotide sequence: SEQ ID NO: 9). SEQ ID NO: 11 corresponds to amino acid 836-844 in SEQ ID NO: 10, SEQ ID NO: 12 corresponds to amino acid 13-29 in SEQ ID NO: 10, SEQ ID NO: 13 corresponds to amino acid 657-681 in SEQ ID NO: 10, SEQ ID NO: 14 corresponds to amino acid 471-479 in SEQ ID NO: 10, SEQ ID NO: 15 corresponds to amino acid 546-555 in SEQ ID NO: 10, and SEQ ID NO: 16 corresponds to amino acid 727-741 in SEQ ID NO: 10.

Accordingly, in the present invention, the antigen in spot 3 can be any of (3A-a) to (3A-e) and (3B) as defined below:
(3A-a) a protein comprising an amino acid sequence with deletion, substitution, insertion or addition of one or several amino acids in SEQ ID NO: 10;
(3A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 10;
(3A-c) a protein comprising an amino acid sequence encoded by a nucleotide sequence with deletion, substitution, insertion or addition of one or several nucleotides in SEQ ID NO: 9;
(3A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 9.
(3A-e) a protein comprising an amino acid sequence encoded by a nucleic acid that hybridizes under stringent conditions with a nucleic acid having a nucleotide sequence complementary to the nucleotide sequence of SEQ ID NO: 9;
(3B) a protein comprising at least one amino acid sequence selected from the group consisting of SEQ ID NOs: 10-16, preferably a protein comprising at least 2, 3, 4, 5, 6 or all sequences of the amino acid sequences. More preferably, a protein comprising at least one amino acid sequence selected from the group consisting of SEQ ID NOs: 10, 12, 13, 15, and 16, preferably a protein comprising at least 2, 3, 4, or all sequences of the amino acid sequences. As referred to above, the amino acid sequence of any of SEQ ID NOs: 10-16 may be an amino acid sequence derived therefrom by deletion, substitution, insertion or addition of one or several amino acids.

The proteins of (3A-a) to (3A-e) and (3B) as defined above also include those proteins whose amino acid residues are modified by phosphorylation, sugar chain modification, aminoacylation, ring-opening, deamination or the like.

The proteins of (3A-a) to (3A-e) and (3B) as defined above can be proteins that are found in a protein spot with a molecular weight of around 80 to 160 kDa, preferably around 90 to 120 kDa and an isoelectric point of 4.0 to 10.0, preferably an isoelectric point of 5.0 to 8.0 on gels used in the two-dimensional electrophoresis performed under the conditions described above in the subsection titled “Identification of antigens”.

Preferably, a protein that is an antigen in spot 3 is an antigen of an allergy to fish.

(4) Antigen in Spot 4

As the result of sequence identification of the antigen in spot 4 by mass spectroscopy, the following amino acid sequences of SEQ ID NOs: 20-24 were detected.

Also, the mass spectroscopic data (SEQ ID NOs: 20-24) obtained for spot 4 on a mass spectrometer was analyzed by comparing the data against the Uniprot protein data, and as a result, the antigen in question was identified as Elongation factor 2 (amino acid sequence: SEQ ID NO: 19, encoding nucleotide sequence: SEQ ID NO: 18). SEQ ID NO: 20 corresponds to amino acid 831-840 in SEQ ID NO: 19, SEQ ID NO: 21 corresponds to amino acid 560-571 in SEQ ID NO: 19, SEQ ID NO: 22 corresponds to amino acid 560-572 in SEQ ID NO: 19, SEQ ID NO: 23 corresponds to amino acid 677-688 in SEQ ID NO: 19, and SEQ ID NO: 24 corresponds to amino acid 620-629 in SEQ ID NO: 19.

Accordingly, in the present invention, the antigen in spot 4 can be any of (4-a) to (4-e) and (4B) as defined below:
(4A-a) a protein comprising an amino acid sequence with deletion, substitution, insertion or addition of one or several amino acids in SEQ ID NO: 19;
(4A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 19;
(4A-c) a protein comprising an amino acid sequence encoded by a nucleotide sequence with deletion, substitution, insertion or addition of one or several nucleotides in SEQ ID NO: 18;
(4A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 18;
(4A-e) a protein comprising an amino acid sequence encoded by a nucleic acid that hybridizes under stringent conditions with a nucleic acid having a nucleotide sequence complementary to the nucleotide sequence of SEQ ID NO: 18;
(4B) a protein comprising at least one amino acid sequence selected from the group consisting of SEQ ID NOs: 19-24, preferably a protein comprising at least 2, 3, 4, 5, 6 or all sequences of the amino acid sequences. As referred to above, the amino acid sequence of any of SEQ ID NOs: 19-24 may be an amino acid sequence derived therefrom by deletion, substitution, insertion or addition of one or several amino acids.

The proteins of (4A-a) to (4A-e) and (4B) as defined above also include those proteins whose amino acid residues are modified by phosphorylation, sugar chain modification, aminoacylation, ring-opening, deamination or the like.

The proteins of (4A-a) to (4A-e) and (4B) as defined above can be proteins that are found in a protein spot with a molecular weight of around 80 to 160 kDa, preferably around 90 to 120 kDa and an isoelectric point of 5.0 to 11.0, preferably an isoelectric point of 6.0 to 8.0 on gels used in the two-dimensional electrophoresis performed under the conditions described above in the subsection titled “Identification of antigens”.

Preferably, a protein that is an antigen in spot 4 is an antigen of an allergy to fish.

(5) Antigen in Spot 5

As the result of sequence identification of the antigen in spot 5 by mass spectroscopy, the following amino acid sequences of SEQ ID NOs: 27-31 were detected.

Also, the mass spectroscopic data (SEQ ID NOs: 27-31) obtained for spot 5 on a mass spectrometer was analyzed by comparing the data against the Uniprot protein data, and as a result, the antigen in question was identified as Heat shock cognate 70 kDa protein (amino acid sequence: SEQ ID NO: 26, encoding nucleotide sequence: SEQ ID NO: 25). SEQ ID NO: 27 corresponds to amino acid 113-126 in SEQ ID NO: 26, SEQ ID NO: 28 corresponds to amino acid 513-526 in SEQ ID NO: 26, SEQ ID NO: 29 corresponds to amino acid 89-102 in SEQ ID NO: 26, SEQ ID NO: 30 corresponds to amino acid 584-597 in SEQ ID NO: 26, and SEQ ID NO: 31 corresponds to amino acid 138-159 in SEQ ID NO: 26.

Accordingly, in the present invention, the antigen in spot 5 can be any of (5A-a) to (5A-e) and (5B) as defined below:
(5A-a) a protein comprising an amino acid sequence with deletion, substitution, insertion or addition of one or several amino acids in SEQ ID NO: 26;
(5A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 26;
(5A-c) a protein comprising an amino acid sequence encoded by a nucleotide sequence with deletion, substitution, insertion or addition of one or several nucleotides in SEQ ID NO: 25;
(5A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 25;
(5A-e) a protein comprising an amino acid sequence encoded by a nucleic acid that hybridizes under stringent conditions with a nucleic acid having a nucleotide sequence complementary to the nucleotide sequence of SEQ ID NO: 25;
(5B) a protein comprising at least one amino acid sequence selected from the group consisting of SEQ ID NOs: 26-31, preferably a protein comprising at least 2, 3, 4, 5 or all sequences of the amino acid sequences. As referred to above, the amino acid sequence of any of SEQ ID NOs: 26-31 may be an amino acid sequence derived therefrom by deletion, substitution, insertion or addition of one or several amino acids.

The proteins of (5A-a) to (5A-e) and (5B) as defined above also include those proteins whose amino acid residues are modified by phosphorylation, sugar chain modification, aminoacylation, ring-opening, deamination or the like.

The proteins of (5A-a) to (5A-e) and (5B) as defined above can be proteins that are found in a protein spot with a molecular weight of around 50 to 110 kDa, preferably around 60 to 90 kDa and an isoelectric point of 3.0 to 7.0, preferably an isoelectric point of 3.0 to 6.0 on gels used in the two-dimensional electrophoresis performed under the conditions described above in the subsection titled “Identification of antigens”.

Preferably, a protein that is an antigen is an antigen of an allergy to fish.

(6) Antigen in Spot 6

As the result of sequence identification of the antigen in spot 6 by mass spectroscopy, the following amino acid sequences of SEQ ID NOs: 34-41 were detected.

Also, the mass spectroscopic data (SEQ ID NOs: 34-41) obtained for spot 6 on a mass spectrometer was analyzed by comparing the data against the Uniprot protein data, and as a result, the antigen in question was identified as Serotransferrin (amino acid sequence: SEQ ID NO: 33, encoding nucleotide sequence: SEQ ID NO: 32). SEQ ID NO: 34 corresponds to amino acid 330-341 in SEQ ID NO: 33, SEQ ID NO: 35 corresponds to amino acid 332-341 in SEQ ID NO: 33, SEQ ID NO: 36 corresponds to amino acid 264-274 in SEQ ID NO: 33, SEQ ID NO: 37 corresponds to amino acid 113-124 in SEQ ID NO: 33, SEQ ID NO: 38 corresponds to amino acid 112-124 in SEQ ID NO: 33, SEQ ID NO: 39 corresponds to amino acid 112-125 in SEQ ID NO: 33, SEQ ID NO: 40 corresponds to amino acid 275-292 in SEQ ID NO: 33, and SEQ ID NO: 41 corresponds to amino acid 436-446 in SEQ ID NO: 33.

Accordingly, in the present invention, the antigen in spot 6 can be any of (6A-a) to (6A-e) and (6B) as defined below:
(6A-a) a protein comprising an amino acid sequence with deletion, substitution, insertion or addition of one or several amino acids in SEQ ID NO: 33;
(6A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 33;
(6A-c) a protein comprising an amino acid sequence encoded by a nucleotide sequence with deletion, substitution, insertion or addition of one or several nucleotides in SEQ ID NO: 32;
(6A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 32;
(6A-e) a protein comprising an amino acid sequence encoded by a nucleic acid that hybridizes under stringent conditions with a nucleic acid having a nucleotide sequence complementary to the nucleotide sequence of SEQ ID NO: 32;
(6B) a protein comprising at least one amino acid sequence selected from the group consisting of SEQ ID NOs: 33-41, preferably a protein comprising at least 2, 3, 4, 5, 6, 7, 8 or all sequences of the amino acid sequences. As referred to above, the amino acid sequence of any of SEQ ID NOs: 33-41 may be an amino acid sequence derived therefrom by deletion, substitution, insertion or addition of one or several amino acids.

The proteins of (6A-a) to (6A-e) and (6B) as defined above also include those proteins whose amino acid residues are modified by phosphorylation, sugar chain modification, aminoacylation, ring-opening, deamination or the like.

The proteins of (6A-a) to (6A-e) and (6B) as defined above can be proteins that are found in a protein spot with a molecular weight of around 60 to 110 kDa, preferably around 70 to 100 kDa and an isoelectric point of 4.0 to 8.0, preferably an isoelectric point of 5.0 to 7.0 on gels used in the two-dimensional electrophoresis performed under the conditions described above in the subsection titled “Identification of antigens”.

Preferably, a protein that is an antigen in spot 6 is an antigen of an allergy to fish.

(7) Antigen in Spot 7

As the result of sequence identification of the antigen in spot 7 by mass spectroscopy, the following amino acid sequences of SEQ ID NOs: 44-54 were detected.

Also, the mass spectroscopic data (SEQ ID NOs: 44-54) obtained for spot 7 on a mass spectrometer was analyzed by comparing the data against the Uniprot protein data, and as a result, the antigen in question was identified as Myosin binding protein H-like (amino acid sequence: SEQ ID NO: 43, encoding nucleotide sequence: SEQ ID NO: 42). SEQ ID NO: 44 corresponds to amino acid 275-292 in SEQ ID NO: 43, SEQ ID NO: 45 corresponds to amino acid 49-66 in SEQ ID NO: 43, SEQ ID NO: 46 corresponds to amino acid 67-77 in SEQ ID NO: 43, SEQ ID NO: 47 corresponds to amino acid 310-323 in SEQ ID NO: 43, SEQ ID NO: 48 corresponds to amino acid 382-401 in SEQ ID NO: 43, SEQ ID NO: 49 corresponds to amino acid 402-418 in SEQ ID NO: 43, SEQ ID NO: 50 corresponds to amino acid 210-219 in SEQ ID NO: 43, SEQ ID NO: 51 corresponds to amino acid 210-228 in SEQ ID NO: 43, SEQ ID NO: 52 corresponds to amino acid 152-183 in SEQ ID NO: 43, SEQ ID NO: 53 corresponds to amino acid 100-112 in SEQ ID NO: 43, and SEQ ID NO: 54 corresponds to amino acid 113-129 in SEQ ID NO: 43.

Accordingly, in the present invention, the antigen in spot 7 can be any of (7A-a) to (7A-e) and (7B) as defined below:
(7A-a) a protein comprising an amino acid sequence with deletion, substitution, insertion or addition of one or several amino acids in SEQ ID NO: 43;
(7A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 43;
(7A-c) a protein comprising an amino acid sequence encoded by a nucleotide sequence with deletion, substitution, insertion or addition of one or several nucleotides in SEQ ID NO: 42;
(7A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 42;
(7A-e) a protein comprising an amino acid sequence encoded by a nucleic acid that hybridizes under stringent conditions with a nucleic acid having a nucleotide sequence complementary to the nucleotide sequence of SEQ ID NO: 42;
(7B) a protein comprising at least one amino acid sequence selected from the group consisting of SEQ ID NOs: 43-54, preferably a protein comprising at least 2, 3, 4, 5, 6, 7, 8, 9, 10, 11 or all sequences of the amino acid sequences; As referred to above, the amino acid sequence of any of SEQ ID NOs: 43-54 may be an amino acid sequence derived therefrom by deletion, substitution, insertion or addition of one or several amino acids.

The proteins of (7A-a) to (7A-e) and (7B) as defined above also include those proteins whose amino acid residues are modified by phosphorylation, sugar chain modification, aminoacylation, ring-opening, deamination or the like.

The proteins of (7A-a) to (7A-e) and (7B) as defined above can be proteins that are found in a protein spot with a molecular weight of around 40 to 80 kDa, preferably around 50 to 60 kDa and an isoelectric point of 4.0 to 8.0, preferably an isoelectric point of 4.0 to 7.0 on gels used in the two-dimensional electrophoresis performed under the conditions described above in the subsection titled “Identification of antigens”.

Preferably, a protein that is an antigen in spot 7 is an antigen of an allergy to fish.

(8) Antigen in Spot 8

As the result of sequence identification of the antigen in spot 8 by mass spectroscopy, the following amino acid sequences of SEQ ID NOs: 57-59 were detected.

Also, the mass spectroscopic data (SEQ ID NOs: 57-59) obtained for spot 8 on a mass spectrometer was analyzed by comparing the data against the Uniprot protein data, and as a result, the antigen in question was identified as Desmin (Fragment) (amino acid sequence: SEQ ID NO: 56, encoding nucleotide sequence: SEQ ID NO: 55). SEQ ID NO: 57 corresponds to amino acid 364-380 in SEQ ID NO: 56, SEQ ID NO: 58 corresponds to amino acid 74-94 in SEQ ID NO: 56, and SEQ ID NO: 59 corresponds to amino acid 273-282 in SEQ ID NO: 56.

Accordingly, in the present invention, the antigen in spot 8 can be any of (8A-a) to (8A-e) and (8B) as defined below:
(8A-a) a protein comprising an amino acid sequence with deletion, substitution, insertion or addition of one or several amino acids in SEQ ID NO: 56;
(8A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 56;
(8A-c) a protein comprising an amino acid sequence encoded by a nucleotide sequence with deletion, substitution, insertion or addition of one or several nucleotides in SEQ ID NO: 55;
(8A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 55;
(8A-e) a protein comprising an amino acid sequence encoded by a nucleic acid that hybridizes under stringent conditions with a nucleic acid having a nucleotide sequence complementary to the nucleotide sequence of SEQ ID NO: 55;
(8B) a protein comprising at least one amino acid sequence selected from the group consisting of SEQ ID NOs: 56-59, preferably a protein comprising at least 2, 3, or all sequences of the amino acid sequences. As referred to above, the amino acid sequence of any of SEQ ID NOs: 56-59 may be an amino acid sequence derived therefrom by deletion, substitution, insertion or addition of one or several amino acids.

The proteins of (8A-a) to (8A-e) and (8B) as defined above also include those proteins whose amino acid residues are modified by phosphorylation, sugar chain modification, aminoacylation, ring-opening, deamination or the like.

The proteins of (8A-a) to (8A-e) and (8B) as defined above can be proteins that are found in a protein spot with a molecular weight of around 40 to 80 kDa, preferably around 50 to 60 kDa and an isoelectric point of 3.0 to 7.0, preferably an isoelectric point of 4.0 to 6.0 on gels used in the two-dimensional electrophoresis performed under the conditions described above in the subsection titled “Identification of antigens”.

Preferably, a protein that is an antigen in spot 8 is an antigen of an allergy to fish.

(9) Antigen in Spot 9

As the result of sequence identification of the antigen in spot 9 by mass spectroscopy, the following amino acid sequences of SEQ ID NOs: 62-68 were detected.

Also, the mass spectroscopic data (SEQ ID NOs: 62-68) obtained for spot 9 on a mass spectrometer was analyzed by comparing the data against the UniProt protein data, and as a result, the antigen in question was identified as Capping protein (Actin filament) muscle Z-line beta (amino acid sequence: SEQ ID NO: 61, encoding nucleotide sequence: SEQ ID NO: 60). SEQ ID NO: 62 corresponds to amino acid 200-223 in SEQ ID NO: 61, SEQ ID NO: 63 corresponds to amino acid 238-254 in SEQ ID NO: 61, SEQ ID NO: 64 corresponds to amino acid 261-274 in SEQ ID NO: 61, SEQ ID NO: 65 corresponds to amino acid 224-235 in SEQ ID NO: 61, SEQ ID NO: 66 corresponds to amino acid 169-181 in SEQ ID NO: 61, SEQ ID NO: 67 corresponds to amino acid 245-254 in SEQ ID NO: 61, and SEQ ID NO: 68 corresponds to amino acid 79-95 in SEQ ID NO: 61.

Accordingly, the antigen in spot 9 in the present invention can be any of the proteins as defined below in (9A-a) to (9A-e), and (9B):
(9A-a) a protein comprising an amino acid sequence with deletion, substitution, insertion or addition of one or several amino acids in SEQ ID NO: 61;
(9A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 61;
(9A-c) a protein comprising an amino acid sequence encoded by a nucleotide sequence with deletion, substitution, insertion or addition of one or several nucleotides in SEQ ID NO: 60;
(9A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 60;
(9A-e) a protein comprising an amino acid sequence encoded by a nucleic acid that hybridizes under stringent conditions with a nucleic acid having a nucleotide sequence complementary to the nucleotide sequence of SEQ ID NO: 60;
(9B) a protein comprising at least one amino acid sequence selected from the group consisting of SEQ ID NOs: 61-68, preferably a protein comprising at least 2, 3, 4, 5, 6, 7 or all sequences of the amino acid sequences. As referred to above, the amino acid sequence of any of SEQ ID NOs: 61-68 may be an amino acid sequence derived therefrom by deletion, substitution, insertion or addition of one or several amino acids;

The proteins as defined above in (9A-a) to (9A-e), and (9B) also include those proteins whose amino acid residues are modified by phosphorylation, sugar chain modification, aminoacylation, ring-opening, deamination or the like.

The proteins as defined above in (9A-a) to (9A-e), and (9B) can be proteins that are found in a protein spot with a molecular weight of around 20 to 50 kDa, preferably around 30 to 40 kDa, and an isoelectric point of 3.0 to 7.0, preferably an isoelectric point of 4.0 to 6.0 on gels used in the two-dimensional electrophoresis performed under the conditions described above in the subsection titled “Identification of antigens”.

Preferably, a protein that is an antigen in spot 9 is an antigen of an allergy to fish.

(10) Antigen in Spot 10

As the result of sequence identification of the antigen in spot 10 by mass spectroscopy, the following amino acid sequences of SEQ ID NOs: 71-108 were detected.

Also, the mass spectroscopic data (SEQ ID NOs: 71-108) obtained for spot 10 on a mass spectrometer was analyzed by comparing the data against the NCBI protein data, and as a result, the antigen in question was identified as myosin heavy chain, fast skeletal muscle-like (amino acid sequence: SEQ ID NO: 70, encoding nucleotide sequence: SEQ ID NO: 69). SEQ ID NO: 71 corresponds to amino acid 13-19 in SEQ ID NO:70, SEQ ID NO: 72 corresponds to amino acid 262-271 in SEQ ID NO: 70, SEQ ID NO: 73 corresponds to amino acid 996-1014 in SEQ ID NO: 70, SEQ ID NO: 74 corresponds to amino acid 951-961 in SEQ ID NO: 70, SEQ ID NO: 75 corresponds to amino acid 1293-1303 in SEQ ID NO: 70, SEQ ID NO: 76 corresponds to amino acid 1082-1091 in SEQ ID NO: 70, SEQ ID NO: 77 corresponds to amino acid 1847-1856 in SEQ ID NO:70, SEQ ID NO: 78 corresponds to amino acid 172-186 in SEQ ID NO: 70, SEQ ID NO: 79 corresponds to amino acid 919-937 in SEQ ID NO: 70, SEQ ID NO: 80 corresponds to amino acid 249-258 in SEQ ID NO: 70, SEQ ID NO: 81 corresponds to amino acid 706-717 in SEQ ID NO: 70, SEQ ID NO: 82 corresponds to amino acid 50-59 in SEQ ID NO: 70, SEQ ID NO: 83 corresponds to amino acid 415-430 in SEQ ID NO:70, SEQ ID NO: 84 corresponds to amino acid 834-845 in SEQ ID NO: 70, SEQ ID NO: 85 corresponds to amino acid 617-630 in SEQ ID NO: 70, SEQ ID NO: 86 corresponds to amino acid 1556-1567 in SEQ ID NO: 70, SEQ ID NO: 87 corresponds to amino acid 1391-1408 in SEQ ID NO: 70, SEQ ID NO: 88 corresponds to amino acid 1025-1040 in SEQ ID NO: 70, SEQ ID NO: 89 corresponds to amino acid 1813-1836 in SEQ ID NO:70, SEQ ID NO: 90 corresponds to amino acid 743-755 in SEQ ID NO: 70, SEQ ID NO: 91 corresponds to amino acid 1172-1192 in SEQ ID NO: 70, SEQ ID NO: 92 corresponds to amino acid 353-364 in SEQ ID NO: 70, SEQ ID NO: 93 corresponds to amino acid 1261-1292 in SEQ ID NO: 70, SEQ ID NO: 94 corresponds to amino acid 1783-1789 in SEQ ID NO: 70, SEQ ID NO: 95 corresponds to amino acid 1502-1519 in SEQ ID NO: 70, SEQ ID NO: 96 corresponds to amino acid 1484-1497 in SEQ ID NO:70, SEQ ID NO: 97 corresponds to amino acid 1194-1212 in SEQ ID NO: 70, SEQ ID NO: 98 corresponds to amino acid 1304-1314 in SEQ ID NO: 70, SEQ ID NO: 99 corresponds to amino acid 369-384 in SEQ ID NO: 70, SEQ ID NO: 100 corresponds to amino acid 1315-1322 in SEQ ID NO: 70, SEQ ID NO: 101 corresponds to amino acid 1536-1555 in SEQ ID NO: 70, SEQ ID NO: 102 corresponds to amino acid 237-248 in SEQ ID NO:70, SEQ ID NO: 103 corresponds to amino acid 1699-1725 in SEQ ID NO: 70, SEQ ID NO: 104 corresponds to amino acid 1092-1104 in SEQ ID NO: 70, SEQ ID NO: 105 corresponds to amino acid 407-414 in SEQ ID NO: 70, SEQ ID NO: 106 corresponds to amino acid 1897-1917 in SEQ ID NO: 70, SEQ ID NO:107 corresponds to amino acid 1458-1470 in SEQ ID NO: 70, and SEQ ID NO: 108 corresponds to amino acid 1373-1388 in SEQ ID NO: 70.

Accordingly, the antigen in spot 10 in the present invention can be any of the proteins as defined below in (10A-a) to (10A-e), and (10B):
(10A-a) a protein comprising an amino acid sequence with deletion, substitution, insertion or addition of one or several amino acids in SEQ ID NO: 70;
(10A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 70;
(10A-c) a protein comprising an amino acid sequence encoded by a nucleotide sequence with deletion, substitution, insertion or addition of one or several nucleotides in SEQ ID NO: 69;
(10A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 69;
(10A-e) a protein comprising an amino acid sequence encoded by a nucleic acid that hybridizes under stringent conditions with a nucleic acid having a nucleotide sequence complementary to the nucleotide sequence of SEQ ID NO: 69;
(10B) a protein comprising at least one amino acid sequence selected from the group consisting of SEQ ID NOs: 70-108, preferably a protein comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or all sequences of the amino acid sequences. As referred to above, the amino acid sequence of any of SEQ ID NOs: 70-108 may be an amino acid sequence derived therefrom by deletion, substitution, insertion or addition of one or several amino acids;

The proteins as defined above in (10A-a) to (10A-e), and (10B) also include those proteins whose amino acid residues are modified by phosphorylation, sugar chain modification, aminoacylation, ring-opening, deamination or the like.

The proteins as defined above in (10A-a) to (10A-e), and (10B) can be proteins that are found in a protein spot with a molecular weight of around 160 to 300 kDa, preferably around 160 to 260 kDa, more preferably around 200 to 230 kDa, and an isoelectric point of 3.0 to 7.0, preferably an isoelectric point of 4.0 to 6.0, more preferably an isoelectric point of 4.5 to 5.5 on gels used in the two-dimensional electrophoresis performed under the conditions described above in the subsection titled “Identification of antigens”.

Preferably, a protein that is an antigen in spot 10 is an antigen of an allergy to fish.

(11) Antigen in Spot 11

As the result of sequence identification of the antigen in spot 11 by mass spectroscopy, the following amino acid sequences of SEQ ID NOs: 111-119 were detected.

Also, the mass spectroscopic data (SEQ ID NOs: 111-119) obtained for spot 11 on a mass spectrometer was analyzed by comparing the data against the NCBI protein data, and as a result, the antigen in question was identified as glycogen phosphorylase, muscle form-like (amino acid sequence: SEQ ID NO: 110, encoding nucleotide sequence: SEQ ID NO: 109). SEQ ID NO: 111 corresponds to amino acid 471-479 in SEQ ID NO: 110, SEQ ID NO: 112 corresponds to amino acid 547-555 in SEQ ID NO: 110, SEQ ID NO: 113 corresponds to amino acid 203-215 in SEQ ID NO:110, SEQ ID NO: 114 corresponds to amino acid 727-741 in SEQ ID NO: 110, SEQ ID NO: 115 corresponds to amino acid 508-520 in SEQ ID NO: 110, SEQ ID NO: 116 corresponds to amino acid 742-755 in SEQ ID NO: 110, SEQ ID NO: 117 corresponds to amino acid 13-29 in SEQ ID NO: 110, SEQ ID NO:118 corresponds to amino acid 775-784 in SEQ ID NO: 110, and SEQ ID NO: 119 corresponds to amino acid 643-656 in SEQ ID NO: 110.

Accordingly, the antigens in spot 11 in the present invention can be any of the proteins as defined below in (11A-a) to (11A-e) and (11B):
(11A-a) a protein comprising an amino acid sequence with deletion, substitution, insertion or addition of one or several amino acids in SEQ ID NO: 110;
(11A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 110;
(11A-c) a protein comprising an amino acid sequence encoded by a nucleotide sequence with deletion, substitution, insertion or addition of one or several nucleotides in SEQ ID NO: 109;
(11A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 109;
(11A-e) a protein comprising an amino acid sequence encoded by a nucleic acid that hybridizes under stringent conditions with a nucleic acid having a nucleotide sequence complementary to the nucleotide sequence of SEQ ID NO: 109;
(11B) a protein comprising at least one amino acid sequence selected from the group consisting of SEQ ID NOs: 110-119, preferably a protein comprising at least 2, 3, 4, 5, 6, 7, 8, 9 or all sequences of the amino acid sequences. As referred to above, the amino acid sequence of any of SEQ ID NOs: 110-119 may be an amino acid sequence derived therefrom by deletion, substitution, insertion or addition of one or several amino acids.

The proteins of (11A-a) to (11A-e) and (11B) as defined above also include those proteins whose amino acid residues are modified by phosphorylation, sugar chain modification, aminoacylation, ring-opening, deamination or the like.

The proteins of (11A-a) to (11A-e) and (11B) as defined above can be proteins that are found in a protein spot with a molecular weight of around 80 to 160 kDa, preferably around 80 to 110 kDa, more preferably around 90 to 110 kDa and an isoelectric point of 4.0 to 8.0, preferably an isoelectric point of 5.0 to 7.5, more preferably an isoelectric point of 6.5 to 7.0 on gels used in the two-dimensional electrophoresis performed under the conditions described above in the subsection titled “Identification of antigens”.

Preferably, a protein that is an antigen in spot 11 is an antigen of an allergy to fish.

(12) Antigen in Spot 12

As the result of sequence identification of the antigen in spot 12 by mass spectroscopy, the following amino acid sequences of SEQ ID NOs: 122-136 were detected.

Also, the mass spectroscopic data (SEQ ID NOs: 122-136) obtained for spot 12 on a mass spectrometer was analyzed by comparing the data against the NCBI protein data, and as a result, the antigen in question was identified as myosin-binding protein C, fast-type-like (amino acid sequence: SEQ ID NO: 121, encoding nucleotide sequence: SEQ ID NO: 120). SEQ ID NO: 122 corresponds to amino acid 197-204 in SEQ ID NO: 121, SEQ ID NO: 123 corresponds to amino acid 421-434 in SEQ ID NO: 121, SEQ ID NO: 124 corresponds to amino acid 329-336 in SEQ ID NO:121, SEQ ID NO: 125 corresponds to amino acid 413-420 in SEQ ID NO: 121, SEQ ID NO:126 corresponds to amino acid 240-246 in SEQ ID NO: 121, SEQ ID NO: 127 corresponds to amino acid 214-228 in SEQ ID NO: 121, SEQ ID NO: 128 corresponds to amino acid 1014-1024 in SEQ ID NO: 121, SEQ ID NO: 129 corresponds to amino acid 842-855 in SEQ ID NO: 121, SEQ ID NO: 130 corresponds to amino acid 260-274 in SEQ ID NO:121, SEQ ID NO: 131 corresponds to amino acid 672-685 in SEQ ID NO: 121, SEQ ID NO: 132 corresponds to amino acid 506-512 in SEQ ID NO: 121, SEQ ID NO: 133 corresponds to amino acid 205-213 in SEQ ID NO: 121, SEQ ID NO: 134 corresponds to amino acid 165-182 in SEQ ID NO: 121, SEQ ID NO:135 corresponds to amino acid 321-328 in SEQ ID NO: 121, and SEQ ID NO: 136 corresponds to amino acid 70-82 in SEQ ID NO: 121.

Accordingly, the antigens in spot 12 in the present invention can be any of the proteins as defined below in (12A-a) to (12A-e) and (12B):
(12A-a) a protein comprising an amino acid sequence with deletion, substitution, insertion or addition of one or several amino acids in SEQ ID NO: 121;
(12A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 121;
(12A-c) a protein comprising an amino acid sequence encoded by a nucleotide sequence with deletion, substitution, insertion or addition of one or several nucleotides in SEQ ID NO: 119;
(12A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 119;
(12A-e) a protein comprising an amino acid sequence encoded by a nucleic acid that hybridizes under stringent conditions with a nucleic acid having a nucleotide sequence complementary to the nucleotide sequence of SEQ ID NO: 119;
(12B) a protein comprising at least one amino acid sequence selected from the group consisting of SEQ ID NOs: 121-136, preferably a protein comprising at least 2, 3, 4, 5, 6, 7, 8, 9, 10, 11, 12, 13, 14, 15 or all sequences of the amino acid sequences. As referred to above, the amino acid sequence of any of SEQ ID NOs: 121-136 may be an amino acid sequence derived therefrom by deletion, substitution, insertion or addition of one or several amino acids.

The proteins of (12A-a) to (12A-e) and (12B) as defined above also include those proteins whose amino acid residues are modified by phosphorylation, sugar chain modification, aminoacylation, ring-opening, deamination or the like.

The proteins of (12A-a) to (12A-e) and (12B) as defined above can be proteins that are found in a protein spot with a molecular weight of around 100 to 160 kDa, preferably around 110 to 150 kDa, more preferably around 120 to 140 kDa and an isoelectric point of 4.0 to 7.0, preferably an isoelectric point of 4.0 to 6.0, more preferably an isoelectric point of 5.0 to 6.0 on gels used in the two-dimensional electrophoresis performed under the conditions described above in the subsection titled “Identification of antigens”. Preferably, a protein that is an antigen in spot 12 is an antigen of an allergy to fish.

(13) Antigen in Spot 13

As the result of sequence identification of the antigen in spot 13 by mass spectroscopy, the following amino acid sequences of SEQ ID NOs: 139-142 were detected.

Also, the mass spectroscopic data (SEQ ID NOs: 139-142) obtained for spot 13 on a mass spectrometer was analyzed by comparing the data against the NCBI protein data, and as a result, the antigen in question was identified as ATP synthase subunit beta, mitochondrial (amino acid sequence: SEQ ID NO: 138, encoding nucleotide sequence: SEQ ID NO: 137). SEQ ID NO: 139 corresponds to amino acid 191-201 in SEQ ID NO: 138, SEQ ID NO: 140 corresponds to amino acid 449-469 in SEQ ID NO: 138, SEQ ID NO:141 corresponds to amino acid 202-214 in SEQ ID NO: 138, and SEQ ID NO: 142 corresponds to amino acid 178-187 in SEQ ID NO: 138.

Accordingly, the antigen in spot 13 in the present invention can be any of the proteins as defined below in (13A-a) to (13A-e), and (13B):
(13A-a) a protein comprising an amino acid sequence with deletion, substitution, insertion or addition of one or several amino acids in SEQ ID NO: 138;
(13A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 138;
(13A-c) a protein comprising an amino acid sequence encoded by a nucleotide sequence with deletion, substitution, insertion or addition of one or several nucleotides in SEQ ID NO: 137;
(13A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 137;
(13A-e) a protein comprising an amino acid sequence encoded by a nucleic acid that hybridizes under stringent conditions with a nucleic acid having a nucleotide sequence complementary to the nucleotide sequence of SEQ ID NO: 137;
(13B) a protein comprising at least one amino acid sequence selected from the group consisting of SEQ ID NOs: 138-142, preferably a protein comprising at least 2, 3, 4 or all sequences of the amino acid sequences. As referred to above, the amino acid sequence of any of SEQ ID NOs: 138-142 may be an amino acid sequence derived therefrom by deletion, substitution, insertion or addition of one or several amino acids;

The proteins as defined above in (13A-a) to (13A-e), and (13B) also include those proteins whose amino acid residues are modified by phosphorylation, sugar chain modification, aminoacylation, ring-opening, deamination or the like.

The proteins as defined above in (13A-a) to (13A-e), and (13B) can be proteins that are found in a protein spot with a molecular weight of around 30 to 70 kDa, preferably around 40 to 60 kDa, more preferably around 45 to 55 kDa and an isoelectric point of 3.0 to 7.0, preferably an isoelectric point of 3.0 to 6.0, more preferably an isoelectric point of 4.0 to 5.5 on gels used in the two-dimensional electrophoresis performed under the conditions described above in the subsection titled “Identification of antigens”.

Preferably, a protein that is an antigen in spot 13 is an antigen of an allergy to fish.

(14) Antigen in Spot 14

As the result of sequence identification of the antigen in spot 14 by mass spectroscopy, the following amino acid sequences of SEQ ID NOs: 145-149 were detected.

Also, the mass spectroscopic data (SEQ ID NOs: 145-149) obtained for spot 14 on a mass spectrometer was analyzed by comparing the data against the NCBI protein data, and as a result, the antigen in question was identified as L-lactate dehydrogenase A chain-like (amino acid sequence: SEQ ID NO: 144, encoding nucleotide sequence: SEQ ID NO: 143). SEQ ID NO: 145 corresponds to amino acid 119-126 in SEQ ID NO: 144, SEQ ID NO: 146 corresponds to amino acid 7-22 in SEQ ID NO: 144, SEQ ID NO: 147 corresponds to amino acid 9-22 in SEQ ID NO: 144, SEQ ID NO: 148 corresponds to amino acid 270-278 in SEQ ID NO: 144, and SEQ ID NO: 149 corresponds to amino acid 91-118 in SEQ ID NO: 144.

Accordingly, the antigens in spot 14 in the present invention can be any of the proteins as defined below in (14A-a) to (14A-e) and (14B):
(14A-a) a protein comprising an amino acid sequence with deletion, substitution, insertion or addition of one or several amino acids in SEQ ID NO: 144;
(14A-b) a protein comprising an amino acid sequence having at least 70%, preferably at least 75%, at least 80%, at least 85%, at least 90%, at least 95%, at least 97%, at least 98%, or at least 99% identity to the amino acid sequence of SEQ ID NO: 144;
(14A-c) a protein comprising an amino acid sequence encoded by a nucleotide sequence with deletion, substitution, insertion or addition of one or several nucleotides in SEQ ID NO: 143;
(14A-d) a protein comprising an amino acid sequence encoded by a nucleotide sequence having at least 70%, preferably at least 75%, at least 80%, at least 85%, at least 90%, at least 95%, at least 97%, at least 98%, or at least 99% identity to the nucleotide sequence of SEQ ID NO: 143;
(14A-e) a protein comprising an amino acid sequence encoded by a nucleic acid that hybridizes under stringent conditions with a nucleic acid having a nucleotide sequence complementary to the nucleotide sequence of SEQ ID NO: 143;
(14B) a protein comprising at least one amino acid sequence selected from the group consisting of SEQ ID NOs: 144-149, preferably a protein comprising at least 2, 3, 4, 5 or all sequences of the amino acid sequences. As referred to above, the amino acid sequence of any of SEQ ID NOs: 144-149 may be an amino acid sequence derived therefrom by deletion, substitution, insertion or addition of one or several amino acids.

The proteins of (14A-a) to (14A-e) and (14B) as defined above also include those proteins whose amino acid residues are modified by phosphorylation, sugar chain modification, aminoacylation, ring-opening, deamination or the like.

The proteins of (14A-a) to (14A-e) and (14B) as defined above can be proteins that are found in a protein spot with a molecular weight of around 20 to 50 kDa, preferably around 30 to 50 kDa, more preferably around 30 to 40 kDa and an isoelectric point of 5.0 to 9.0, preferably an isoelectric point of 6.0 to 8.0, more preferably an isoelectric point of 6.5 to 7.5 on gels used in the two-dimensional electrophoresis performed under the conditions described above in the subsection titled “Identification of antigens”.

Preferably, a protein that is an antigen in spot 14 is an antigen of an allergy to fish.

By stating herein “deletion, substitution, insertion or addition of one or several amino acids” in relation to amino acid sequence, it is meant that in an amino acid sequence of interest, one or several amino acids (e.g., 30%, preferably 25%, 20%, 15%, 10%, 5%, 3%, 2%, or 1% of amino acids with respect to the total length of the amino acid sequence) are deleted, one or several amino acids are substituted by any other amino acids, any other amino acids are inserted, and/or any other amino acids are added.

Among the aforementioned modifications, substitution is preferably conservative substitution. The “conservative substitution” refers to the substitution of a certain amino acid residue by a different amino acid residue having similar physicochemical characteristics, and can be any type of substitution as long as it does not substantially change the characteristics of the structure of the original sequence for example, any type of substitution is acceptable as long as any substituted amino acids do not disrupt the helical structure of the original sequence or other secondary structures that characterize the original sequence. The following gives examples of separate groups of amino acid residues that are conservatively substitutable with each other, but substitutable amino acid residues are not limited to the examples given below.
Group A: leucine, isoleucine, valine, alanine, methionine
Group B: aspartic acid, glutamic acid
Group C: asparagine, glutamine
Group D: lysine, arginine
Group E: serine, threonine
Group F: phenylalanine, tyrosine

In the case of non-conservative substitution, one member belonging to one of the aforementioned groups can be replaced with a member belong to any other group. For example, in order to eliminate the possibility of unwanted sugar-chain modification, amino acids of group B, D or E as listed above may be substituted by those of any other group. Also, cysteines may be deleted or substituted by any other amino acids to prevent them from being folded into a protein in its tertiary structure. Also, in order to maintain the balance between hydrophilicity and hydrophobicity or to increase hydrophilicity for the purpose of facilitating synthesis, any amino acids may be substituted in consideration of the hydropathy scales of amino acids, which are a measure of the hydrophilic and hydrophobic properties of amino acids (J. Kyte and R. Doolittle, J. Mol. Biol., Vol. 157, p. 105-132, 1982).

As referred to herein, the percent identity between two amino acid sequences can be determined by visual inspection and mathematical calculation. Alternatively, the percent identity can be determined using a computer program. Examples of such computer programs include BLAST and ClustalW. In particular, various conditions (parameters) for identity searches with the BLAST program are described in Altschul, et al. (Nucl. Acids. Res., 25, p. 3389-3402, 1997), and are publicly available on the websites of the National Center for Biotechnology Information (NCBI) and DNA Data Bank of Japan (DDBJ) (Altschul, et al., BLAST Manual, Altschul, et al., NCB/NLM/NIH Bethesda, MD 20894). Also, the percent identity can be determined using a genetic information processing software program, such as GENETYX Ver.7 (Genetyx Corporation), DINASIS Pro (Hitachi Software Engineering Co., Ltd.), or Vector NTI (Infomax Inc.).

By stating herein “deletion, substitution, insertion or addition of one or several nucleotides” in relation to nucleotide sequence, it is meant that in a nucleotide sequence of interest, one or several nucleotides (e.g., 30%, preferably 25%, 20%, 15%, 10%, 5%, 3%, 2% or 1%, of nucleotides with respect to the total length of the nucleotide sequence) are deleted, one or several nucleotides are substituted by any other nucleotides, any other nucleotides are inserted, and/or any other nucleotides are added. It is preferable that such a nucleotide deletion, substitution, insertion or addition should not give rise to a frame shift in an amino acid coding sequence.

As referred to herein, the percent identity between two nucleotide sequences can be determined by visual inspection and mathematical calculation. Alternatively, the percent identity can be determined using a computer program. Examples of such sequence comparison computer programs include the BLASTN program, version 2.2.7, available on the website of the National Library of Medicine (http://www.ncbi.nlm nih.gov/blast/b12seq/b1s.html) (Altschul, et al. (1990) J. Mol. Biol., 215: 403-10), or the WU-BLAST 2.0 algorithm. Standard default parameter settings for WU-BLAST 2.0 are found and available on the following website: http://blast.wustl.edu.

As referred to herein, “under stringent conditions” means that hybridization takes place under moderately or highly stringent conditions. To be specific, the moderately stringent conditions can be easily determined by those having ordinary skill in the art on the basis of, for example, the length of DNA. Basic conditions are described in Sambrook, et al., Molecular Cloning: A Laboratory Manual, 3rd ed., ch. 6-7, Cold Spring Harbor Laboratory Press, 2001. The moderately stringent conditions include hybridization under the conditions of preferably 1×SSC to 6×SSC at 42° C. to 55° C., more preferably 1×SSC to 3×SSC at 45° C. to 50° C., most preferably 2×SSC at 50° C. In the case of using a hybridization solution containing, for example, about 50% formamide, a temperature around 5 to 15° C. lower than the foregoing should be adopted. Washing is also carried out under the conditions of 0.5×SSC to 6×SSC at 40° C. to 60° C. In the process of hybridization and washing, generally 0.05% to 0.2% SDS, preferably about 0.1% SDS, may be added. Likewise, the highly stringent conditions can be easily determined by those having ordinary skill in the art on the basis of, for example, the length of DNA. Generally, the highly stringent (high stringent) conditions include hybridization and/or washing at a higher temperature and/or a lower salt concentration than those adopted under the moderately stringent conditions. For example, hybridization is carried out under the conditions of 0.1×SSC to 2×SSC at 55° C. to 65° C., more preferably 0.1×SSC to 1×SSC at 60° C. to 65° C., most preferably 0.2×SSC at 63° C. Washing is carried out under the conditions of 0.2×SSC to 2×SSC at 50° C. to 68° C., more preferably 0.2×SSC at 60 to 65° C.

Antigens may be obtained by separating and purifying them from fish using a combination of protein purification methods well known to those skilled in the art. Also, antigens may be obtained by expressing them as recombinant proteins using a genetic recombination technique well known to those skilled in the art and by separating and purifying them using protein purification methods well known to those skilled in the art.

Exemplary protein purification methods include: solubility-based purification methods such as salt precipitation and solvent precipitation; purification methods based on molecular weight difference, such as dialysis, ultrafiltration, gel filtration and SDS-PAGE; charge-based purification methods such as ion exchange chromatography and hydroxylapatite chromatography; specific affinity-based purification methods such as affinity chromatography; purification methods based on hydrophobicity difference, such as reverse-phase high-performance liquid chromatography; and purification methods based on isoelectric point difference, such as isoelectric focusing.

Preparation of a protein by a genetic recombination technique is carried out by preparing an expression vector comprising an antigen-encoding nucleic acid, introducing the expression vector into appropriate host cells by gene transfer or genetic transformation, culturing the host cells under suitable conditions for expression of a recombinant protein, and recovering the recombinant protein expressed in the host cells.

The “vector” refers to a nucleic acid that can be used to introduce a nucleic acid attached thereto into host cells. The “expression vector” is a vector that can induce the expression of a protein encoded by a nucleic acid introduced thereby. Exemplary vectors include plasmid vectors, viral vectors, and the like. Those skilled in the art can select an appropriate expression vector for the expression of a recombinant protein depending on the type of host cells to be used. To facilitate purification, an affinity tags such as 6 His residues may be included. Moreover, a protein may be synthesized so that a signal sequence is included and the protein is secreted out of cells.

The “host cells” refers to cells that undergo gene transfer or genetic transformation by a vector. The host cells can be appropriately selected by those skilled in the art depending on the type of the vector to be used. The host cells can be derived from prokaryotes such as Escherichia coli (E. coli). When prokaryotic cells like E. coli are used as host cells, the antigen of the present invention may be designed to contain an N-terminal methionine residue in order to facilitate the expression of a recombinant protein in the prokaryotic cells. The N-terminal methionine can be cleaved from the recombinant protein after expression. Also, the host cells may be cells derived from eukaryotes, such as single-cell eukaryotes like yeast, plant cells and animal cells (e.g., human cells, monkey cells, hamster cells, rat cells, murine cells or insect cells) or silkworm.

Gene transfer or genetic transformation of an expression vector into host cells can be carried out as appropriate by following a technique known to those skilled in the art. Those skilled in the art can make possible the expression of a recombinant protein by selecting suitable conditions for the expression of the recombinant protein as appropriate depending on the type of host cells and culturing the host cells under the selected conditions. Then, those skilled in the art can homogenize the host cells having the expressed recombinant protein, and separate and purify an antigen expressed as the recombinant protein from the resulting homogenate by using an appropriate combination of such protein purification methods as mentioned above.

Diagnostic Kit and Method

The present invention provides a method for providing an indicator for diagnosing a fish allergy in a subject, the method comprising the steps of:
(i) contacting a sample obtained from the subject with an antigen, wherein the sample is a solution comprising an IgE antibody;
(ii) detecting binding between an IgE antibody present in the sample obtained from the subject and the antigen; and
(iii) when the binding between the IgE antibody in the subject and the antigen is detected, an indicator of the fact that the subject has a fish allergy is provided;
   wherein the antigen is at least one of the proteins identified as the aforementioned antigens (1) to (9) or at least one of the proteins identified as the aforementioned antigens (10) to (14).

The sample obtained from a subject is a solution containing an IgE antibody, as collected from the subject. Examples of such solutions include blood, saliva, sputum, snivel, urine, sweat, and tear. The sample obtained from the subject may be subjected to pretreatment for increasing the concentration of an IgE antibody in the sample before being contacted with an antigen. The pretreatment of a sample may involve, for example, collection of the serum and the plasma from the blood. Furthermore, Fab portions that are binding portions with the antigens may be purified. In a particularly preferred embodiment, the step (i) mentioned above is carried out by contacting an IgE antibody present in the serum obtained from a subject with an antigen.

The IgE antibody may be the IgE antibody itself or mast cells or the like to which the IgE antibody is bound.

Detection of contact and binding between the sample obtained from a subject and an antigen can be carried out by using a known method. Examples of such methods that can be used include detection by ELISA (Enzyme-Linked Immunosorvent Assay), sandwich immunoassay, immunoblotting, immunoprecipitation, or immunochromatography. These are all techniques involving contacting and binding an IgE antibody from a subject with an antigen and detecting the IgE antibody specifically bound to the antigen. Examples thereof include methods involving bringing an enzyme-labeled secondary antibody in contact, adding a substrate (usually a chromogenic or luminescent reagent) of the enzyme, and detecting the product of the enzymatic reaction; methods involving bringing a biotinylated secondary antibody in contact, adding an avidin-conjugated dye, and detecting the dye; and methods involving detecting a fluorescently labeled secondary antibody. Also, detection by a measurement method that permits the evaluation of binding between an antigen and an IgE antibody, such as surface plasmon resonance (SPR), can be used. A plurality of antigen-specific IgE antibodies may be mixed.

The antigen may be provided as an isolated antigen in a state immobilized on a carrier. In this case, the steps (i) and (ii) mentioned above can be carried out using ELISA, sandwich immunoassay, immunochromatography, surface plasmon resonance, or the like. Also, the step (i) mentioned above is carried out by contacting the sample obtained from a subject with an antigen-immobilized surface. The isolated antigen may be obtained by separating and purifying it from fish using a combination of protein purification methods well known to those skilled in the art, or by preparing it using a genetic recombination technique. Moreover, the antibody that binds to the antigen may be in an immobilized state.

The antigen may be in an unimmobilized state to a carrier. In this case, the steps (i) and (ii) mentioned above can be carried out by flow cytometry or the like and the presence of the antigen bound to an antibody may be confirmed using a laser beam. Examples include basophil activation test (BAT) in which an antigen binds to an antibody and the amount of the surface antigen CD203c which is expressed when basophils in the sample are activated is measured, and the like. Another example is histamine release test (HRT) in which it is examined whether histamine is released or not by bringing an antigen in contact with hemocytes via the binding to an antibody in a sample.

The antigen may be transferred from a state separated by two-dimensional electrophoresis and detected by immunoblotting. The two-dimensional electrophoresis is a technique for separating a protein sample by performing isoelectric focusing in the first dimension and performing SDS-PAGE (SDS-polyacrylamide gel electrophoresis) in the second dimension. The conditions for two-dimensional electrophoresis are not particularly limited as long as the conditions permit the separation of the antigen of the present invention. For example, the conditions for two-dimensional electrophoresis as described above in the subsection titled “Identification of antigens” can be adopted. Also, the electrophoresis conditions may be determined by reference to the descriptions in PTLs 1 to 4 mentioned above. For example, two-dimensional electrophoresis can be carried out under the conditions that satisfy at least one selected from the group consisting of the following requirements:
(A) the isoelectric focusing gels used in the first dimension should have a gel-strip length of 5 to 10 cm and a gel pH range of 3 to 10, and the pH gradient of the gels in the direction of electrophoresis should be as follows: where the gel-strip length up to pH 5 is taken as “a”, that length from pH 5 to 7 as “b”, and that length above pH 7 as “c”, the relations “a<b” and “b>c” are satisfied;
(B) in the case of (A), when the total gel-strip length is taken as 1, “a” should be in the range of 0.15 to 0.3, “b” should be in the range of 0.4 to 0.7, and “c” should be in the range of 0.15 to 0.3;
(C) in the first dimensional isoelectric focusing, a constant voltage step should be performed by applying a constant voltage ranging from 100 V to 600 V per gel strip containing a sample, and after the electrophoresis variation width during electrophoresis for 30 minutes falls within the range of 5 μA, a voltage-increasing step should be started at which the voltage is increased from the aforementioned constant voltage;
(D) in the case of (C), the final voltage at the voltage-increasing step should be in the range of 3000 V to 6000 V;
(E) the isoelectric focusing gels used in the first dimension should have a longitudinal gel-strip length of 5 to 10 cm, and the electrophoresis gels used in the second dimension should have a gel concentration at the distal end in the direction of electrophoresis, which is in the range of 3 to 6%; and
(F) in the case of (E), the electrophoresis gels used in the second dimension should have a gel concentration at the proximal end in the direction of electrophoresis, which is set to a higher value than that at the distal end in the direction of electrophoresis.

The aforementioned antigens (1) to (9) and the aforementioned antigens (10) to (14) are antigens which specifically bind to IgE antibodies in patients with an allergy to fish. Therefore, when binding between an IgE antibody from a subject and the antigen is detected, an indicator of the fact that the subject has a fish allergy is provided.

The present invention further provides a kit for diagnosing an allergy to a fish, comprising at least one of the aforementioned antigens (1) to (9) or at least one of the aforementioned antigens (10) to (14). The diagnostic kit of this invention may be used in the aforementioned method for providing an indicator for diagnosing an allergy to a fish or in a diagnostic method as described later. The diagnostic kit of this invention comprises at least one of the aforementioned antigens (1) to (9) or at least one of the aforementioned antigens (10) to (14) and may comprise a chromogenic substrate or luminescent substrate serving as a substrate of an enzyme-labeled anti-IgE antibody and the enzyme or a biotinylated anti-IgE antibody and an avidin-conjugated dye that binds to the biotin. Also, a fluorescent-labeled anti-IgE antibody may be used. In the diagnostic kit of this invention, the antigen may be provided in a state immobilized on a carrier. The diagnostic kit of this invention may also be provided together with instructions on the procedure for diagnosis or a package containing said instructions.

In another embodiment, the diagnostic kit comprises a companion diagnostic agent for an allergy to fish. The companion diagnostic agent is used to analyze the reactivity of a pharmaceutical product for the purpose of identifying a patient in which the pharmaceutical product is expected to work or a patient at risk for serious side effects from the pharmaceutical product, or optimizing a therapy using the pharmaceutical product. Here, examples of optimizing a therapy include determination of the dosage and administration, decision of stopping the administration, determination of the allergen component to be used for the induction of immune tolerance, and the like.

The present invention further provides a composition for diagnosing an allergy to a fish, comprising at least one of the aforementioned antigens (1) to (9) or at least one of the aforementioned antigens (10) to (14). The diagnostic composition of this invention can be used in a diagnostic method as described below. The diagnostic composition of this invention may further comprise a pharmaceutically acceptable carrier and/or additives commonly used with the antigen of this invention depending on the need.

In one embodiment, the present invention provides a method for diagnosing an allergy to a fish in a subject, the method comprising:
(i) contacting a sample obtained from the subject with an antigen;
(ii) detecting binding between an IgE antibody present in the sample obtained from the subject and the antigen; and
(iii) when the binding between the IgE antibody in the subject and the antigen is detected, diagnosing the subject as being allergic to a fish;
   wherein the antigen is at least one of the proteins identified as the aforementioned antigens (1) to (9) or at least one of the proteins identified as the aforementioned antigens (10) to (14). In this method, each of the steps (i) and (ii) is performed as described above regarding each of the corresponding steps of the method for providing an indicator for diagnosing an allergy to a fish.

In another embodiment, the present invention provides a method for diagnosing an allergy to a fish in a subject, the method comprising administering to the subject at least one of the aforementioned antigens (1) to (9) or at least one of the aforementioned antigens (10) to (14). This method may be performed in the form of a skin test characterized by applying the antigen onto the skin Examples of the skin test include various forms of tests, such as: a prick test in which a diagnostic composition is applied onto the skin and then a tiny prick to such an extent as not to provoke bleeding is made in the skin to allow an antigen to infiltrate into the skin, thereby observing a skin reaction; a scratch test in which a diagnostic composition is applied onto the skin and then the skin is lightly scratched to observe a reaction; a patch test in which a diagnostic composition in the form of cream, ointment, etc. is applied onto the skin to observe a reaction; and an intracutaneous test in which an antigen is administered intracutaneously to observe a reaction. The method for infiltrating a diagnostic composition into the skin in a prick test or a scratch test may be a method involving bringing a tip of a lancet in contact with a diagnostic composition and wounding the skin by pricking the skin with the contact site to infiltrate the composition. If a skin reaction such as swelling occurs in a skin portion to which the antigen has been applied, the subject of interest is diagnosed as having an allergy to a fish. The amount of the antigen to be applied to the skin in such tests can be, for example, not more than 100 μg per dose.

In the allergic diagnosis, a load test for the purpose of identifying the antigen is often performed. At least one of the aforementioned antigens (1) to (9) or at least one of the aforementioned antigens (10) to (14) may be used as an active ingredient of a load test for diagnosing an allergy to fish. Here, the antigen protein to be used in the load test may be a protein that has been expressed and purified and may be a protein that has been expressed in a food and a food material, for example, rice-based vaccine produced by transforming rice with a gene of a cedar pollen antigen to express the antigen protein therein.

In yet another embodiment, the present invention provides at least one of the aforementioned antigens (1) to (9) or at least one of the aforementioned antigens (10) to (14) for use in the diagnosis of an allergy to a fish. Here, the present invention encompasses the provision of at least one of the aforementioned antigens (1) to (9) or (10) to (14) mixed with a known antigen.

In still another embodiment, the present invention provides a use of at least one of the aforementioned antigens (1) to (9) or at least one of the aforementioned antigens (10) to (14) for the production of a diagnostic agent for an allergy to a fish.

Pharmaceutical Composition and Treatment Method

The present invention provides a pharmaceutical composition comprising at least one of the aforementioned antigens (1) to (9) or at least one of the aforementioned antigens (10) to (14).

In one embodiment, the aforementioned pharmaceutical composition is used for the treatment of an allergy to fish.

The present invention also provides a method for treating an allergy to fish, the method comprising administering at least one of the aforementioned antigens (1) to (9) or at least one of the aforementioned antigens (10) to (14) to a patient in need of a treatment for an allergy to a fish.

In another embodiment, the present invention provides at least one of the aforementioned antigens (1) to (9) or at least one of the aforementioned antigens (10) to (14) for use in the treatment of an allergy to fish. In yet another embodiment, the present invention provides use of at least one of the aforementioned antigens (1) to (9) or at least one of the aforementioned antigens (10) to (14) for the production of a therapeutic agent for an allergy to fish.

In the process of allergy treatment, a hyposensitization therapy aiming at inducing immunological tolerance by administering an antigen to a patient is often adopted. At least one of the aforementioned antigens (1) to (9) or at least one of the aforementioned antigens (10) to (14) can be used as an active ingredient for a hyposensitization therapy for allergy to fish. Here, the antigen protein to be used in the hyposensitization therapy may be a protein that has been expressed and purified and may be a protein that has been expressed in a food and a food material, for example, rice-based vaccine produced by transforming rice with a gene of a cedar pollen antigen to express the antigen protein therein.

The pharmaceutical composition of the present invention can be administered by common administration routes. Examples of common administration routes include oral, sublingual, percutaneous, intracutaneous, subcutaneous, intravascular, intranasal, intramuscular, intraperitoneal, and intrarectal administrations.

The pharmaceutical composition of the present invention can be used as a pharmaceutical composition to which a commonly used pharmaceutically acceptable adjuvant or excipient or any other additives (e.g., stabilizer, solubilizer, emulsifier, buffer, preservative, colorant) are added by a conventional method together with the antigen of this invention depending on the need. The dosage form of the pharmaceutical composition can be selected by those skilled in the art as appropriate depending on the administration route. The pharmaceutical composition can be in the form of, for example, tablet, capsule, troche, sublingual tablet, parenteral injection, intranasal spray, poultice, solution, cream, lotion, or suppository. The administration dose, frequency and/or period of the pharmaceutical composition of this invention can be selected by a physician as appropriate depending on the administration route and the patient's condition and characteristics such as age and body weight. For example, the pharmaceutical composition may be administered to an adult patient at a dose of not more than 100 μg per dose. The administration interval can be, for example, once daily, once weekly, twice monthly, once per three months or so. The administration period can be, for example, several weeks to several years. The pharmaceutical composition may be administered in such a manner that the dose is increased in incremental steps over the administration period.

Tester

The present invention provides a tester comprising an antibody for at least one of the aforementioned antigens (1) to (9) or at least one of the aforementioned antigens (10) to (14).

The antibody can be prepared by a conventional method. For example, the antibody may be prepared by immunizing a mammal such as rabbit with one of the aforementioned antigens (1) to (9) or one of the aforementioned antigens (10) to (14). The antibody may be an IgE antibody, a polyclonal antibody, a monoclonal antibody, or an antigen-binding fragment thereof (e.g., Fab, F(ab′)2, Fab′).

Further, in the aforementioned tester, the antibody may be provided in a form bound to a carrier. The type of the carrier is not particularly limited as long as it is usable for detection of binding between an antibody and an antigen. Any given carrier known to those skilled in the art can be used.

Examples of a method for determining the presence or absence of an antigen include:
A method in which a prepared tester comprising an IgE antibody is contacted with a sample obtained from a food or food material, and ELISA or the like method is, for example, used to detect whether there is a binding between the IgE antibody and an antigen in the sample, and if the binding between the IgE antibody and the antigen is detected, it is determined that the antibody remains in the food or food material, of interest.
A method in which proteins are extracted from a food or food material, and the proteins are subjected to electrophoresis, and it is detected with an antibody whether there is a spot band of the antigen.
A method in which a food or food material is infiltrated into a filter paper or the like and an antibody solution is reacted with the filter paper to detect an antigen present in the food.

The present invention encompasses, in another embodiment, a tester for determining the presence or absence of an antigen for an allergy to fish in an object of interest, the tester comprising a primer having a nucleotide sequence complementary to at least one part of one of the nucleotide sequences set forth in SEQ ID NO: 1, 4, 9, 18, 25, 32, 42, 55, 60, 69, 109, 120, 137, or 143. Without limiting, the primer has a nucleotide sequence complementary to preferably 12 residues, 15 nucleotides, 20 nucleotides, or 25 nucleotides in the sequence of a 3′ terminal or middle portion of at least one part of one sequence of the nucleotide sequences set forth in, for example, SEQ ID NO: 1, 4, 9, 18, 25, 32, 42, 55, 60, 69, 109, 120, 137, or 143. Particularly when mRNA is targeted, the tester comprises a poly A tail-complementarity primer. In a preferred embodiment, the tester comprising the primer may further comprise a primer comprising a nucleotide sequence of a 5′ terminal portion of at least one sequence of the nucleotide sequences set forth in SEQ ID NO: 1, 4, 9, 18, 25, 32, 42, 55, 60, 69, 109, 120, 137, or 143, preferably a nucleotide sequence consisting of 12 nucleotides, 15 nucleotides, 20 nucleotides, 25 nucleotides.

For example, the presence or absence of the antigen is determined by amplifying DNA or cDNA by polymerase chain reaction (PCR) including RT-PCR by using the aforementioned primer with DNA or mRNA obtained from fish as a template and comparing the amplified DNA or cDNA sequence(s) with SEQ ID NO: 1, 4, 9, 18, 25, 32, 42, 55, 60, 69, 109, 120, 137, or 143. Examples of the method of the amplification of mRNA by PCR include RACE and the like. In the determination, the antigen is determined to be present when the comparison between the amplified DNA or cDNA and SEQ ID NO: 1, 4, 9, 18, 25, 32, 42, 55, 60, 69, 109, 120, 137, or 143 indicates the presence of point mutation(s) encoding the same amino acid(s), or when the amino acid sequence encoded by the amplified DNA or cDNA has 70% or more, preferably 80, 90, 95, 98, or 99% or more identity with an amino acid sequence of SEQ ID NO: 2, 5, 10, 19, 26, 33, 43, 56, 61, 70, 110, 121, 138, or 144, even if the nucleotide sequence of the amplified DNA or cDNA is modified from any of the nucleotide sequences of SEQ ID NO: 1, 4, 9, 18, 25, 32, 42, 55, 60, 69, 109, 120, 137, or 143 by insertion, deletion, substitution, or addition of nucleotide(s).

In one embodiment, the aforementioned tester is used to determine the presence or absence of an antigen in food materials (fish or fish eggs) or in products of interest in a food production line. The tester may also be used for quality inspection of production lines and pre-shipment products by manufacturers, or may be used for self-checking of the presence or absence of an antigen in a food or food material of interest by consumers.

Allergen-Free Food and the Like

The present invention provides fish or fish eggs in which at least one of the aforementioned antigens (1) to (9) or at least one of the aforementioned antigens (10) to (14) is eliminated or reduced, a processed product of the fish or the fish eggs or fish which lay the fish eggs or have hatched from the eggs.

The method for eliminating or reducing the antigen of the present invention in fish, fish eggs, the processed product of the fish or the fish eggs or the fish which lay the fish eggs or have hatched from the eggs is not limited. The elimination or reduction of the antigen of the present invention may be conducted by any method, as long as the method permits the elimination or reduction of the antigen.

For example, the fish or fish eggs in which the antigen of the present invention is eliminated or reduced may be obtained by preparing fish or fish eggs in which the expression of the antigen of the present invention is knocked out, using a gene knock-out technique.

As the gene knock-out technique, there can be used any methods known to those skilled in the art. For example, Oishi, et al. (Scientific Reports, Vol. 6, Article number: 23980, 2016, doi:10.1038/srep23980) describes that the genome editing technique CRISPER/Cas9 is applied to primordial germ cells to obtain individual animals deficient in an allergen gene. The fish or fish eggs devoid of the antigen of this invention may also be obtained by using the same technique. Moreover, fish or fish eggs in which the antigen of the present invention is eliminated or reduced may be obtained by mating by artificial insemination with fish or fish eggs that do not contain the antigen or contains a low content of the antigen. The artificial mating of fish or fish eggs can be performed by a conventional method.

The processed product of the fish or fish eggs in which the antigen of the present invention is eliminated or reduced may be a processed product made from fish in which the antigen of the present invention is eliminated or reduced. In the case of using ordinary fish or fish eggs as a source ingredient, a treatment for removing or reducing the antigen of the present invention is performed before or after preparation of a processed product of fish or fish eggs. Examples of a method of eliminating or reducing the antigen of the present invention in a processed product of fish or fish eggs made from ordinary fish or fish eggs include methods for eliminating the protein component in a food or a food material, such as elution with a high pressure treatment and a neutral salt solution and hot steam and methods for hydrolysis, denaturation, or amino acid modification (chemical modification or elimination of side chains) by heat treatment and acid treatment. The fish in which the antigen of the present invention is eliminated or reduced may be fish that have hatched and grown from the aforementioned fish eggs in which the antigen of the present invention is eliminated or reduced. Moreover, the fish eggs in which the antigen of the present invention is eliminated or reduced may be those obtained from the aforementioned fish in which the antigen of the present invention is eliminated or reduced.

### EXAMPLES

The following describes examples of the present invention. The technical scope of this invention is not limited by these examples.

### Example 1: Confirmation of a Protein Pattern

Proteins contained in salmon were investigated using a two-dimensional electrophoresis method described below.

Protein Extraction

The extraction and the purification of proteins contained in salmon were performed as follows. A solubilization agent (Mammalian Lysis Buffer (MCLI), SIGMA) was added to flesh of salmon and proteins were extracted, and then a urea buffer was added to obtain a protein extract. The composition of the urea buffer is as follows.
30 mM Tris
2 M Thiourea
7 M Urea
4% (w/v) CHAPS:
3-[(3-cholamidopropyl)dimethylammonio]propanesulfonate

Moderate Amount of Dilute Hydrochloric Acid

Distilled water was added and the total volume was adjusted to 100 mL. pH was 8.5.

Subsequently, 25 μg each in terms of protein weight was mixed to obtain an extract.

Thereafter, the precipitation procedure was repeated twice using a 2D-CleanUP Kit (produced by GE). In the first round of precipitation, the collected liquid protein extract was precipitated by adding TCA (trichloroacetic acid) thereto and the precipitated product produced by this procedure (TCA-precipitated product) was collected. In the second round of precipitation, the TCA-precipitated product collected above was further precipitated by adding acetone thereto and the precipitated product (sample) produced by this procedure was collected.

Preparation of a Sample Solution

Part of the collected sample (40 μg on a protein weight basis) was dissolved in 150 μL of a DeStreak Rehydration Solution (produced by GE), which is a swelling buffer for first-dimensional isoelectric focusing gels, thereby obtaining a sample solution for first-dimensional isoelectric focusing (sample solution for swelling). The constituents of the DeStreak Rehydration Solution are as mentioned below.
7M thiourea
2M urea
4% (w/v) of CHAPS
0.5% (v/v) IPG buffer; produced by GE
Moderate amount of BPB (bromophenol blue)

Impregnation of the Sample into First-Dimensional Isoelectric Focusing Gels

First-dimensional isoelectric focusing gel strips (Immobiline Drystrip IPG gels (pH3-10NL); produced by GE) were immersed in 140 μL of the foregoing sample solution for first-dimensional isoelectric focusing (sample solution for swelling) and impregnated with the solution at room temperature overnight.

In this example, an IPGphor electrophoresis system produced by GE was used.

An electrophoresis tray was filled with silicone oil. Filter paper moisten with water was positioned at both ends of the gel strips impregnated with the sample, and the gel strips were set in the electrophoresis tray such that the gel strips were covered with silicone oil. Electrodes were placed on the gel strips with the filter paper intervening therebetween.

The maximum current of the isoelectric focusing system was set to 75 μA per gel strip, and the first-dimensional isoelectric focusing was carried out according to the following voltage program: (1) a constant voltage step was performed at a constant voltage of 300 V until the volt-hours reached 750 Vhr (the current variation width during electrophoresis for 30 minutes before the end of this step was 5 μA); (2) the voltage was increased gradually to 1000 V for 300 Vhr; (3) the voltage was further increased gradually to 5000 V for 4500 Vhr; and then (4) the voltage was held at a constant voltage of 5000 V until the total Vhr reached 12000.

SDS Equilibration of Isoelectric Focusing Gels

After the aforementioned first-dimensional isoelectric focusing was done, the gel strips were taken out of the isoelectric focusing system, immersed in an equilibration buffer containing a reducing agent, and shaken at room temperature for 15 minutes. The constituents of the equilibration buffer containing the reducing agent are as mentioned below.
100 mM Tris-HCl (pH 8.0)
6M urea
30% (v/v) glycerol
2% (w/v) SDS
1% (w/v) DTT

Next, the equilibration buffer containing the reducing agent was removed, and then the gel strips were immersed in an equilibration buffer containing an alkylating agent and shaken at room temperature for 15 minutes to obtain SDS-equilibrated gels. The constituents of the equilibration buffer containing the alkylating agent are as mentioned below.
100 mM Tris-HCl (pH 8.0)
6M urea
30% (v/v) glycerol
2% (w/v) SDS
2.5% (w/v) iodoacetamide

Second-Dimensional SDS-PAGE

In this example, the XCell SureLock Mini-Cell electrophoresis system produced by Life Technologies was used. The second-dimensional electrophoresis gels used were NuPAGE 4-12% Bis-Tris Gels produced by Life Technologies. Also, an electrophoresis buffer composed of the following constituents was prepared and used.
50 mM MOPS
50 mM Tris base
0.1% (w/v) SDS
1 mM EDTA

Further, an agarose solution for gel adhesion was used in this example, which was prepared by dissolving 0.5% (w/v) Agarose S (produced by Nippon Gene Co., Ltd.) and a moderate amount of BPB (bromophenol blue) in the electrophoresis buffer.

SDS-PAGE wells were washed well with the electrophoresis buffer, and then the buffer used for the washing was removed. Next, the washed wells were charged with the fully dissolved agarose solution for gel adhesion. Next, the SDS-equilibrated gel strips were immersed in agarose and closely adhered to second-dimensional electrophoresis gels using tweezers. After it was confirmed that agarose was fully fixed with the gels being closely adhered to each other, electrophoresis was performed at a constant voltage of 200 V for about 45 minutes.

Fluorescent Staining of Gels

The gels were fluorescently stained with SYPRO Ruby (produced by Life Technologies).

First, an airtight container to be used was washed well in advance with 98% (v/v) ethanol. The electrophoresed second-dimensional electrophoresis gel strips were taken out of the SDS-PAGE system, placed onto the washed airtight container, and treated twice by immersion in 50% (v/v) methanol and 7% (v/v) aqueous solution containing acetic acid for 30 minutes. Then, a further immersion treatment was done for 10 minutes, with the solution being replaced by water. Next, the second-dimensional electrophoresis gel strips were immersed in 40 mL of SYPRO Ruby and shaken at room temperature overnight.

Thereafter, the SYPRO Ruby was removed, and then the second-dimensional electrophoresis gel strips were washed with water and shaken in 10% (v/v) methanol and 7% (v/v) aqueous solution containing acetic acid for 30 minutes. Further shaking was done for at least 30 minutes, with the solution being replaced by water.

Analysis

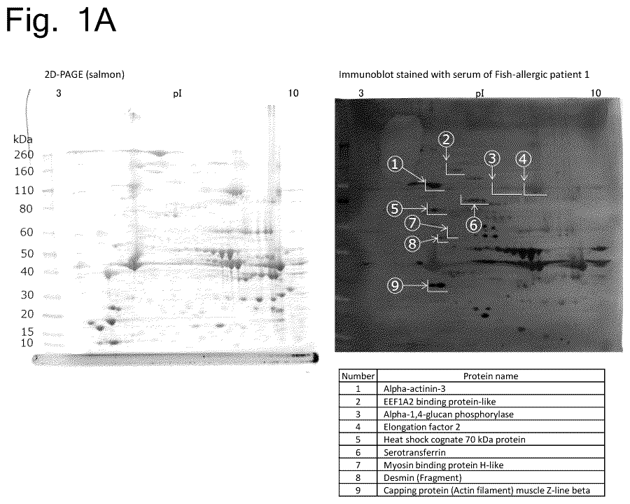

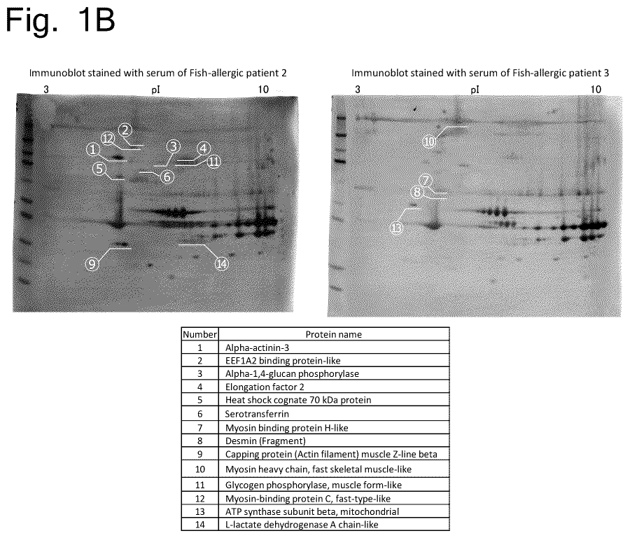

The second-dimensional electrophoresis gels obtained through the foregoing series of treatments were subjected to fluorescent image scanning on Typhoon 9500 (produced by GE). The left panel of FIG. 1A illustrates the result of two-dimensional electrophoresis of proteins contained in flesh of salmon. Molecular weight marker bands are found at the left of the panels. The positions of the bands indicate particular molecular weights (KDa).

### Example 2: Identification of Antigens by Immunoblotting (1)

Identification of antigens by immunoblotting was carried out by taking all the steps up to the step of “Second-dimensional SDS-PAGE” as described above in Example 1, followed by the steps of “Transfer to membrane”, “Immunoblotting” and “Analysis” as described below.

Transfer to Membrane

Transfer to membrane was done using the following transfer system and transfer buffer.

Transfer system: XCell SureLock Mini-Cell and XCell II Blot Module (produced by Life Technologies)
Transfer buffer: NuPAGE Transfer Buffer (X20) (produced by Life Technologies), used in a form diluted 20-fold with milliQ water.

To be specific, proteins in the second-dimensional electrophoresis gels were transferred to a membrane (PVDF membrane) according to the following procedure.
(1) The PVDF membrane was immersed in 100% methanol followed by milliQ water, and then moved into the transfer buffer to hydrophilize the PVDF membrane.
(2) After sponge, filter paper, the second-dimensional electrophoresis gels prepared by second-dimensional SDS-PAGE, the hydrophilized PVDF membrane, filter paper, and sponge were put in place in this order, the transfer system was energized at a constant voltage of 30 V for one hour.

Immunoblotting

Immunoblotting of the membrane was carried out using, as a primary antibody, a serum sample from a patient 1 with a fish allergy or a serum sample from a non-fish-allergic subject. This Fish-allergic patient 1 is a patient diagnosed as immediate-type allergy to fish. This patient developed allergic symptoms to salmon, horse mackerel, sea eel, black porgy, mackerel, sea bream, cod, and young yellowtail and was positive in a prick test.

Immunoblotting of the membrane was carried out according to the following procedure.
(1) The transferred membrane was shaken in a 5% skim milk/PB ST solution (a PBS buffer containing 0.1% Tween 20 nonionic surfactant) at room temperature for one hour.
(2) The membrane was left to stand in a solution of 5% serum as a primary antibody in 5% skim milk/PBST at room temperature for one hour.
(3) The membrane was washed with a PBST solution (5 min×3 times).
(4) The membrane was left to stand in a solution of 1:5000 dilution of the secondary antibody, anti-human IgE-HRP (horseradish peroxidase), with a 5% skim milk/PBST solution at room temperature for one hour.
(5) The membrane was washed with a PBST solution (5 min×3 times).
(6) The membrane was left to stand in Pierce Western Blotting Substrate Plus (produced by Thermo Fisher Scientific) for 5 minutes.

Analysis

The membrane obtained through the foregoing series of treatments was subjected to fluorescent image scanning on Typhoon 9500 (produced by GE).

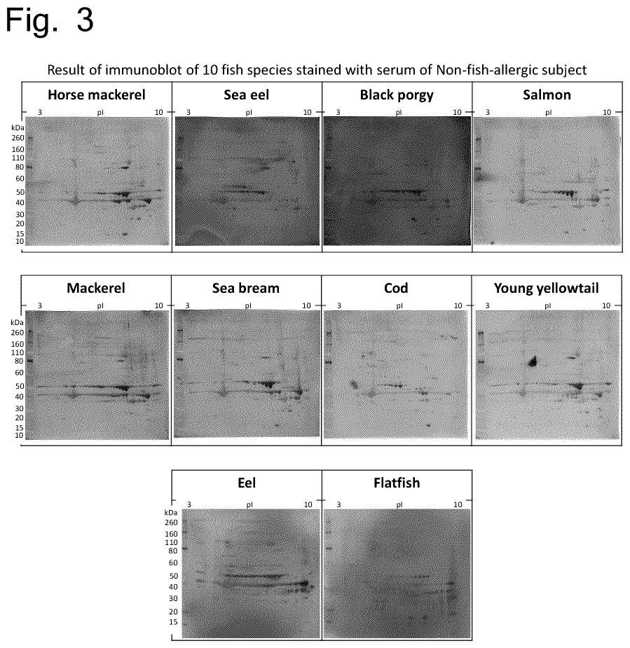

The immunoblot obtained with the serum from Fish-allergic patient 1 was compared with that obtained with the control serum from Non-fish-allergic subject. Nine spots that are different from those with the serum of Non-fish-allergic subject (FIG. 3) and different from the known salmon allergen proteins were detected on an immunoblot of the proteins contained in flesh of salmon with the serum of Fish-allergic patient 1 (FIG. 1A, right panel). The detected spots are indicated in the immunoblot (FIG. 1A, right panel).

The molecular weights and isoelectric points of the 9 spots are as follows.
Spot 1: Molecular weight 90 to 120 kDa, pI 3.0 to 6.0
Spot 2: Molecular weight 120 to 160 kDa, pI 4.0 to 7.0
Spot 3: Molecular weight 90 to 120 kDa, pI 5.0 to 8.0
Spot 4: Molecular weight 90 to 120 kDa, pI 6.0 to 8.0
Spot 5: Molecular weight 60 to 90 kDa, pI 3.0 to 6.0
Spot 6: Molecular weight 70 to 100 kDa, pI 5.0 to 7.0
Spot 7: Molecular weight 40 to 70 kDa, pI 4.0 to 7.0
Spot 8: Molecular weight 40 to 70 kDa, pI 4.0 to 7.0
Spot 9: Molecular weight 20 to 50 kDa, pI 4.0 to 7.0

### Example 3: Mass Spectrometry and Identification of Antigens (1)

The amino acid sequences of the antigens that form the above nine protein spots were identified by mass spectroscopy.

To be specific, protein extraction and mass spectroscopy were done by the following procedure.

(1) Flesh of salmon was subjected to protein extraction, two-dimensional electrophoresis and transfer to membrane by following the procedures described in Example 1 and 2, and the resulting membrane was stained by shaking in a solution of 0.008% Direct blue in 40% ethanol and 10% acetic acid.

(2) Then, the membrane was decolorized by repeating a 5-minute treatment with 40% ethanol and 10% acetic acid three times, washed with water for 5 minutes, and then dried by air.

(3) A protein spot of interest was cut out with a clean cutter blade and put into a centrifugal tube. The cut membrane was subjected to hydrophilization with 50 μL of methanol, followed by washing with 100 μL of water twice and then centrifugal cleaning. Thereafter, 20 μL of 20 mM NH₄HCO₃ and 50% acetonitrile were added.

(4) 1 μL of 1 μmol/μL lysyl endopeptidase (produced by WAKO) was added, and the solution was left to stand at 37° C. for 60 minutes and then collected in a new centrifugal tube. After 20 μL of 20 mM NH₄HCO₃ and 70% acetonitrile were added to the membrane, the membrane was immersed therein at room temperature for 10 minutes, and the resulting solution was further collected. The solution was dissolved with 0.1% formic acid and 10 μL of 4% acetonitrile and transferred to a tube.

(5) The collected solution was dried under reduced pressure, dissolved with 15 μL of solution A (a 0.1% formic acid/4% acetonitrile solution), and analyzed by mass spectroscopy (ESI-TOF6600, produced by AB Sciex).

(6) Identification of proteins based on the MS data obtained with the mass spectrometer was done by searching NCBI or UniProt.

Results

The mass spectrometry of the spots resulted in the detection of the following amino acid sequences.
Spot 1: the amino acid sequences set forth in SEQ ID NO: 3
Spot 2: the amino acid sequences set forth in SEQ ID NO: 6 to 8
Spot 3: the amino acid sequences set forth in SEQ ID NO: 11 to 17
Spot 4: the amino acid sequences set forth in SEQ ID NO: 20 to 24
Spot 5: the amino acid sequences set forth in SEQ ID NO: 27 to 31
Spot 6: the amino acid sequences set forth in SEQ ID NO: 34 to 41
Spot 7: the amino acid sequences set forth in SEQ ID NO: 44 to 54
Spot 8: the amino acid sequences set forth in SEQ ID NO: 57 to 59
Spot 9: the amino acid sequences set forth in SEQ ID NO: 62 to 68

Furthermore, the spots were identified as the following proteins by the analysis of the MS data obtained from the mass spectrometer for spot 1 at NCBI and for spots 2 to 9 with UniProt.
Spot 1: Alpha-actinin-3 (NCBI protein accession number XP_014051545.1, DNA accession number XM_014196070.1) (amino acid sequence: SEQ ID NO: 2, encoding nucleotide sequence: SEQ ID NO: 1)
Spot 2: EEF1A2 binding protein-like (UniProt protein accession number B5RI29, DNA accession number ACH85270.1) (amino acid sequence: SEQ ID NO: 5, encoding nucleotide sequence: SEQ ID NO: 4)
Spot 3: Alpha-1,4-glucan phosphorylase (UniProt protein accession number B5DG55, DNA accession number ACH70729.1) (amino acid sequence: SEQ ID NO: 10, encoding nucleotide sequence: SEQ ID NO: 9)
Spot 4: Elongation factor 2 (UniProt protein accession number C0H9N2, DNA accession number ACN10751.1) (amino acid sequence: SEQ ID NO: 19, encoding nucleotide sequence: SEQ ID NO: 18)
Spot 5: Heat shock cognate 70 kDa protein (UniProt protein accession number B5X3U6, DNA accession number ACI33977.1) (amino acid sequence: SEQ ID NO: 26, encoding nucleotide sequence: SEQ ID NO: 25)
Spot 6: Serotransferrin (UniProt protein accession number P79815, DNA accession number BAA13759.1) (amino acid sequence: SEQ ID NO: 33, encoding nucleotide sequence: SEQ ID NO: 32)
Spot 7: Myosin binding protein H-like (UniProt protein accession number B5DG45, DNA accession number ACH70719.1) (amino acid sequence: SEQ ID NO: 43, encoding nucleotide sequence: SEQ ID NO: 42)
Spot 8: Desmin (Fragment) (UniProt protein accession number Q8UWF1, DNA accession number CAC83053.1) (amino acid sequence: SEQ ID NO: 56, encoding nucleotide sequence: SEQ ID NO: 55)
Spot 9: Capping protein (Actin filament) muscle Z-line beta (UniProt protein accession number B5DFX6, DNA accession number ACH70650.1) (amino acid sequence: SEQ ID NO: 61, encoding nucleotide sequence: SEQ ID NO: 60)

### Example 4: Confirmation of Antigens in Other Fish Species (1)

Using flesh of the 9 fish species, horse mackerel, sea eel, black porgy, mackerel, sea bream, cod, young yellowtail, eel, and flatfish, other than salmon, antigens were confirmed by the same procedure as the procedure described in Example 1 and Example 2.

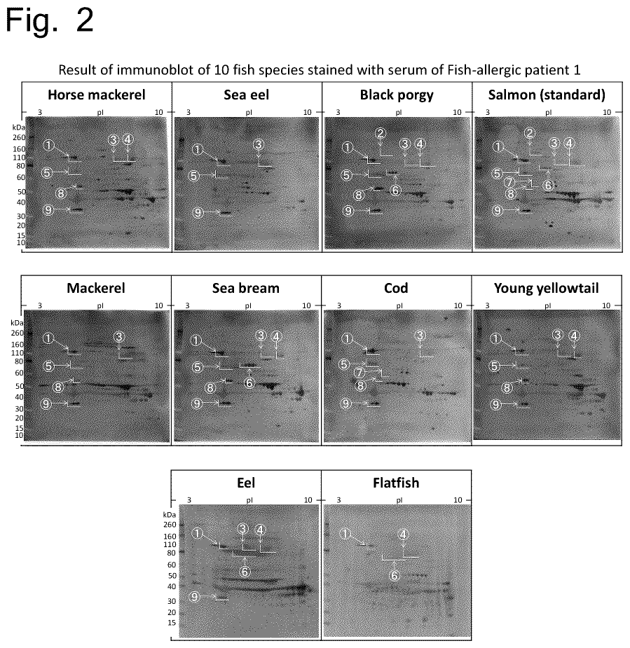

Immunoblots obtained with the serum from Fish-allergic patient 1 was compared with that obtained with the control serum from Non-fish-allergic subject. Spots that are different from those with the serum from Non-fish-allergic subject (FIG. 3) and correspond to a part of the 9 spots obtained in salmon were detected on immunoblots (FIG. 2) of proteins contained in flesh of each of the 9 fish species stained with the serum from Fish-allergic patient 1.

Spots detected in each fish species are as follows.

Horse mackerel: Spots 1, 3-5, 8, and 9

### Sea eel: Spots 1, 3, 5, and 9

Black porgy: Spots 1-6, 8, and 9

### Mackerel: Spots 1, 3, 5, 8, and 9

Sea bream: Spots 1, 3-6, 8, and 9

### Cod: Spots 1, 3, 5, 7-9

Young yellowtail: Spots 1, 3-5, 8, 9

### Eel: Spots 1, 3, 4, 6, 9

### Flatfish: Spots 1, 4, 6

### Example 5: Confirmation of Antigens by Immunoblotting (2)

An immunoblot obtained with serum from a fish-allergic patient was compared with that obtained with the control serum from Non-fish-allergic subject in the same way as in Example 2. Three new spots (Spots 11, 12, 14) that are different from those with the serum from Non-fish-allergic subject (FIG. 3) and different from the known salmon allergen proteins, other than 7 (spots 1-6, 9) of the 9 spots detected in Example 2, were detected on an immunoblot (FIG. 1B, left panel) of proteins contained in flesh of salmon stained with the serum from Fish-allergic patient 2. The total 10 spots detected are indicated in the immunoblot (FIG. 1B, left panel).

The molecular weights and isoelectric points of the 3 new spots detected are as follows.
Spot 11: Molecular weight 90 to 110, pI 6.5 to 7.0
Spot 12: Molecular weight 120 to 140, pI 5.0 to 6.0
Spot 14: Molecular weight 30 to 40, pI 6.5 to 7.5

### Example 6: Mass Spectrometry and Identification of Antigens (2)

The amino acid sequences of the antigens that form the 3 spots newly detected were identified by mass spectroscopy in the same way as in Example 3.

The mass spectrometry of the spots resulted in the detection of the following amino acid sequences.
Spot 11: Amino acid sequences set forth in SEQ ID NOs: 111 to 119
Spot 12: Amino acid sequences set forth in SEQ ID NOs: 122 to 136
Spot 14: Amino acid sequences set forth in SEQ ID NOs: 145 to 149

Furthermore, the spots were identified as the following proteins by the analysis of the MS data obtained from the mass spectrometer for the spots at NCBI.
Spot 11: Glycogen phosphorylase, muscle form-like (NCBI protein accession number XP_013984904.1, DNA accession number XM_014129429.1) (amino acid sequence: SEQ ID NO: 110, encoding nucleotide sequence: SEQ ID NO: 109)
Spot 12: Myosin-binding protein C, fast-type-like (NCBI protein accession number XP_014014310.1, DNA accession number XM_014158835.1) (amino acid sequence: SEQ ID NO: 121, encoding nucleotide sequence: SEQ ID NO: 120)
Spot 14: L-lactate dehydrogenase A chain-like (NCBI protein accession number XP_014003141.1, DNA accession number XM_014147666.1) (amino acid sequence: SEQ ID NO: 144, encoding nucleotide sequence: SEQ ID NO: 143)

### Example 7: Confirmation of Antigens by Immunoblotting (3)

An immunoblot obtained with the serum from a fish-allergic patient was compared with that obtained with the control serum from Non-fish-allergic subject, in the same way as in Example 2. Two new spots (Spots 10, 13) that are different from those with the serum from Non-fish-allergic subject (FIG. 3) and different from the known salmon allergen proteins, other than 2 (Spots 7, 8) of the 9 spots detected in Example 2, were detected on an immunoblot (FIG. 1B, right panel) of proteins contained in flesh of salmon stained with the serum from Fish-allergic patient 3. The total 4 spots detected are indicated in the immunoblot (FIG. 1B, right panel).

The molecular weights and isoelectric points of the 2 new spots detected are as follows.
Spot 10: Molecular weight 200 to 230, pI 4.5 to 5.5
Spot 13: Molecular weight 45 to 55, pI 4.0 to 5.5

### Example 8: Mass Spectrometry and Identification of Antigens (3)

The amino acid sequences of the antigens that form the 2 spots newly detected were identified by mass spectroscopy in the same way as in Example 3.

The mass spectrometry of the spots resulted in the detection of the following amino acid sequences.
Spot 10: Amino acid sequences set forth in SEQ ID NOs: 71 to 108
Spot 13: Amino acid sequences set forth in SEQ ID NOs: 139 to 142

Furthermore, the spots were identified as the following proteins by the analysis of the MS data obtained from the mass spectrometer for the spots at NCBI.
Spot 10: Myosin heavy chain, fast skeletal muscle-like (NCBI protein accession number XP_014039990.1, DNA accession number XM_014184515.1) (amino acid sequence: SEQ ID NO: 70, encoding nucleotide sequence: SEQ ID NO: 69)
Spot 13: ATP synthase subunit beta, mitochondrial (NCBI protein accession number XP_014007238.1, DNA accession number XM_014151763.1) (amino acid sequence: SEQ ID NO: 138, encoding nucleotide sequence: SEQ ID NO: 137)

### Example 9: Confirmation of Antigens in Other Fish Species (2)

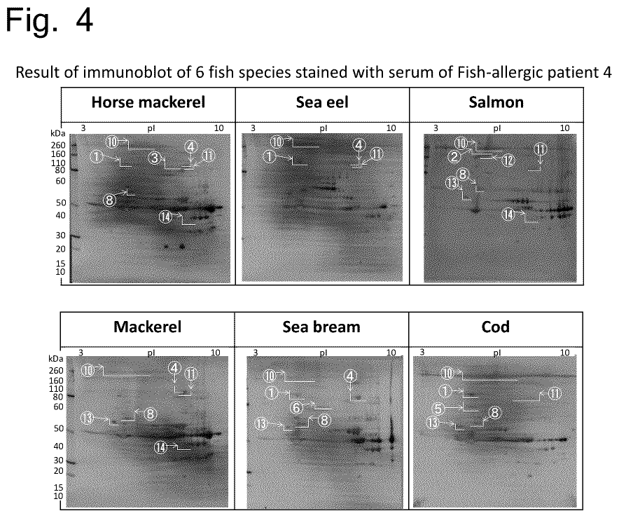

Using flesh of the 6 fish species, horse mackerel, sea eel, salmon, mackerel, sea bream, and cod, the antigen was confirmed in the same way as in Example 4. Immunoblots obtained with the serum from Fish-allergic patient 4 was compared with that obtained with the control serum from Non-fish-allergic subject. Spots that are different from those with the serum from Non-fish-allergic subject (FIG. 3) and correspond to a part of the 14 spots obtained in salmon with the serum from Fish-allergic patients 1 to 3 were detected on immunoblots (FIG. 4) of proteins contained in flesh of each of the 6 fish species stained with the serum from Fish-allergic patient 4.

Spots detected in each fish species are as follows.

Horse mackerel: Spots 1, 3, 4, 8, 10, 11, and 14

### Sea eel: Spots 1, 4, 10, and 11

### Salmon: Spots 2, 8, and 10-14

### Mackerel: Spots 4, 8, 10, 11, 13, and 14

Sea bream: Spots 1, 4, 6, 8, 10, and 13

### Cod: Spots 1, 5, 8, 10, 11, and 13

### INDUSTRIAL APPLICABILITY

The present invention can provide novel antigens of an allergy to fish, methods and kits for diagnosing an allergy to fish, a pharmaceutical composition comprising the antigen, and fish or fish eggs in which the antigen is eliminated or reduced, a processed product of the fish or fish eggs, or fish which lay the fish eggs or have hatched from the eggs. The present invention can further provide a tester for determining the presence or absence of a fish antigen in an object of interest.

1-10. (canceled)

11. A method for detecting binding between an antigen and an IgE antibody present in a sample,
wherein the antigen is defined as: an Alpha-actinin-3 protein or a variant thereof, wherein said Alpha-actinin-3 protein or variant thereof is isolated and immobilized to a carrier or a surface, is capable of binding specifically to an IgE antibody of a patient with an allergy to fish, and is defined below in any of (a) to (d):
(a) comprising the amino acid sequence of SEQ ID NO: 2, with or without a conserved amino acid substitution of a residue of SEQ ID NO: 2;
(b) having 100% identity to the full-length amino acid sequence of SEQ ID NO: 2;
(c) comprising an amino acid sequence encoded by a nucleotide sequence having 100% identity to the nucleotide sequence of SEQ ID NO: 1; or
(d) encoded by a nucleic acid that hybridizes over its full length under high stringent conditions of 0.1×SSC to 2×SSC hybridization at 55° C. to 65° C. and/or 0.2×SSC to 2×SSC washing at 50° C. to 68° C., with the nucleic acid complementary to the nucleotide sequence of SEQ ID NO: 1,
the method comprising a step of contacting an antigen and an IgE antibody present in a sample by using a kit that comprises an antigen(s) which is at least one of said polypeptide wherein said antigen(s) is immobilized to a carrier or a surface.

12. A method for providing an indicator for diagnosing a fish allergy in a subject, wherein said allergy is an allergy to an antigen which is a polypeptide as defined in claim 11,
the method comprising a step of detecting binding between said antigen and an IgE antibody present in a sample obtained from the subject by the method according to claim 11 using a kit that comprises an antigen(s) which is at least one of the polypeptide as defined in claim 11, wherein said antigen(s) is immobilized to a carrier or a surface, wherein an indicator of the fact that the subject has a fish allergy is provided by detection of the binding between the IgE antibody in the subject and the antigen.

13. A method for diagnosing fish allergy in a subject, wherein said allergy is an allergy to an antigen which is a polypeptide as defined in claim 11,
the method comprising a step of detecting binding between said antigen and an IgE antibody present in a sample obtained from the subject by the method according to claim 11 using a kit that comprises an antigen(s) which is at least one of the polypeptide as defined in claim 11, wherein said antigen(s) is immobilized to a carrier or a surface, wherein binding between the IgE antibody present in the sample and the antigen is indicative that the subject is allergic to fish.

14. A method for detecting binding between an antigen and an IgE antibody present in a sample,
wherein the antigen is defined as a Heat shock cognate 70 kDa protein or a variant thereof, wherein said Heat shock cognate 70 kDa protein or variant thereof is isolated and immobilized to a third carrier or a third surface, is capable of binding specifically to an IgE antibody of a patient with an allergy to fish, and is defined below in any of (a) to (d):
(a) comprising the amino acid sequence of SEQ ID NO: 26, with or without a conserved amino acid substitution of a residue of SEQ ID NO: 26;
(b) having 100% identity to the full-length amino acid sequence of SEQ ID NO: 26;
(c) comprising an amino acid sequence encoded by a nucleotide sequence having 100% identity to the nucleotide sequence of SEQ ID NO: 25; or
(d) encoded by a nucleic acid that hybridizes over its full length under high stringent conditions of 0.1×SSC to 2×SSC hybridization at 55° C. to 65° C. and/or 0.2×SSC to 2×SSC washing at 50° C. to 68° C., with the nucleic acid complementary to the nucleotide sequence of SEQ ID NO: 25,
the method comprising a step of contacting an antigen and an IgE antibody present in a sample by using a kit that comprises an antigen(s) which is at least one of said polypeptide, wherein said antigen(s) is immobilized to a carrier or a surface.

15. A method for providing an indicator for diagnosing a fish allergy in a subject, wherein said allergy is an allergy to an antigen which is a polypeptide as defined in claim 14,
the method comprising a step of detecting binding between said antigen and an IgE antibody present in a sample obtained from the subject by the method according to claim 14 using a kit that comprises an antigen(s) which is at least one of the polypeptide as defined in claim 14, wherein said antigen(s) is immobilized to a carrier or a surface, wherein an indicator of the fact that the subject has a fish allergy is provided by detection of the binding between the IgE antibody in the subject and the antigen.

16. A method for diagnosing fish allergy in a subject, wherein said allergy is an allergy to an antigen which is a polypeptide as defined in claim 14,
the method comprising a step of detecting binding between said antigen and an IgE antibody present in a sample obtained from the subject by the method according to claim 14 using a kit that comprises an antigen(s) which is at least one of the polypeptide as defined in claim 14, wherein said antigen(s) is immobilized to a carrier or a surface, wherein binding between the IgE antibody present in the sample and the antigen is indicative that the subject is allergic to fish.

17. A method for detecting binding between an antigen and an IgE antibody present in a sample,
wherein the antigen is defined as: a Capping protein (Actin filament) muscle Z-line beta protein or a variant thereof, wherein said Capping protein (Actin filament) muscle Z-line beta protein or variant thereof is isolated and immobilized to a third carrier or a third surface, is capable of binding specifically to an IgE antibody of a patient with an allergy to fish, and is defined below in any of (a) to (d):
(a) comprising the amino acid sequence of SEQ ID NO: 61, with or without a conserved amino acid substitution of a residue of SEQ ID NO: 61;
(b) having 100% identity to the full-length amino acid sequence of SEQ ID NO: 61;
(c) comprising an amino acid sequence encoded by a nucleotide sequence having 100% identity to the nucleotide sequence of SEQ ID NO: 60; or
(d) encoded by a nucleic acid that hybridizes over its full-length under high stringent conditions of 0.1×SSC to 2×SSC hybridization at 55° C. to 65° C. and/or 0.2×SSC to 2×SSC washing at 50° C. to 68° C., with the nucleic acid complementary to the nucleotide sequence of SEQ ID NO: 60,
the method comprising a step of contacting an antigen and an IgE antibody present in a sample by using a kit that comprises an antigen(s) which is at least one of said polypeptide, wherein said antigen(s) is immobilized to a carrier or a surface.

18. A method for providing an indicator for diagnosing a fish allergy in a subject, wherein said allergy is an allergy to an antigen which is a polypeptide as defined in claim 17,
the method comprising a step of detecting binding between said antigen and an IgE antibody present in a sample obtained from the subject by the method according to claim 17 using a kit that comprises an antigen(s) which is at least one of the polypeptide as defined in claim 17, wherein said antigen(s) is immobilized to a carrier or a surface, wherein an indicator of the fact that the subject has a fish allergy is provided by detection of the binding between the IgE antibody in the subject and the antigen.

19. A method for diagnosing fish allergy in a subject, wherein said allergy is an allergy to an antigen which is a polypeptide as defined in claim 17,
the method comprising a step of detecting binding between said antigen and an IgE antibody present in a sample obtained from the subject by the method according to claim 17 using a kit that comprises an antigen(s) which is at least one of the polypeptide as defined in claim 17, wherein said antigen(s) is immobilized to a carrier or a surface, wherein binding between the IgE antibody present in the sample and the antigen is indicative that the subject is allergic to fish.


SEQUENCE LISTING (149 sequences)

SEQ ID NO: 1 - DNA, 2685 bp, synthetic construct
  misc_feature 1..2685: note = PREDICTED: alpha-actinin-3
  source 1..2685: mol_type = other DNA
atgacggcaatcgaaactcaggtgcaatatggctcctacatgacttcggagcaagtctacatgacccaagaggatgattgggacagggaccttctgttggaccctgcctgggagaaacagcagcgcaagaccttcaccgcctggtgcaactctcacctgcgtaaagcgggcacacagattgagaacattgaggaggacttcaggaatgggctcaagctcatgttgctgttagaggtcatctcaggtgaaaggcttcccaaaccagacaaaggcaaaatgcgtttccacaaaatcgccaacgtgaacaaggccctggatttcatctgcagcaagggagtcaagctggtgtccatcggtgccgaggagattgtggatggtaatgtgaagatgaccctggggatgatctggaccatcattctgcgtttcgccatccaggacatctctgtagaggagacctctgctaaggagggtctgttgctgtggtgccagaggaagactgccccctacaggaatgtcaatgtgcagaacttccacatcagttggaaggatggcctggcactgtgtgccctcatccacagacacagacctgacctcattgactactccaaactgcgcaaggatgaccccatgggcaatctcaacactgcctttgaggtggcagagaagtacctggacatccccaagatgttggatgcagaagatattgtgaacaccccgaagcccgacgagaaagccatcatgacctatgtgtcctgcttctatcatgccttcgctggagccgagcaggccgagacagctgccaataggatctgcaaggtgttggctgtcaaccaggagaacgagaagctcatggaggagtatgagaagctggccagtgagctgctggagtggatccgtcgcaccatcccctggctggagaaccgcgtggctgagcagaccatgcgcgccatgcagcagaagctggaggacttccgtgactaccgtcgcgtccacaagcctcccaaggtgcaggagaagtgtcagctggagattaacttcaacaccctgcagaccaagctgaggctgagcaacaggcccgccttcatgccctccgagggcaagatggtgtcggacattgccaatgcctggaagggtctggagcaggtagagaagggctatgaggagtggctgctcactgagatcagacgcctggagaggctggaccacctggctgagaagttcaagcagaagtcttccctgcatgagtcctggacctcaggtaaggtggagctgctgtccatgaaggactatgagtctgcctcactgatggagatccgtgccctgatgaggaagcacgaggcgtttgagagcgacctggctgcccaccaggacagagtggagcagattgctgccatcgcccaggagctcaatgagctggactatcatgatgccgtcaccatcaacgcccgctgccagggtatttgtgaccagtgggacaacctgggcaccttgacccagaagaggagagactcactggagcgtgtggagaagctgtgggagaccatcgaccagctgtacctggagtttgccaagagagcagcccccttcaacaactggatggacggagccatggaggacttacaggacatgttcattgtgcacagcattgaggagatccagagtctgatcacagcccatgaccagttcaaggccaccctgccagaggcagataaggagcgcgttgcaaccatgggaatccagaatgagatcgtgaagatcgctcagacctacggcatcaagctgtcaggagtcaacccctacaccaacctttccccccaggacatcaccgacaaatgggatgctgtgaaacacctggtgcccctcagggatcagatgctgcaggaggaggtggccaggcagcagtccaacgagaggctgaggcgccagttcgctgcccaggccaacatcatcggaccctggatccagaccaagatggaggagatcagccatgtgtctgtggacatcgccggctccctggaggaacagatgagcaacctgaagcagtacgagcagaacatcatcaactacaagtgcaacatcgacaagctggagggagaccaccaactcagccaggagtccctcatctttgacaacaagcacaccaactacaccatggagcatgtgcgtgtgggctgggagcagctcctcaccaccatcgcccgcaccatcaacgaggtggagaaccagatcctgacccgagatgccaagggcatcagccaggagcagctcaacgagttcagagcctccttcaaccactttgacaggaagagaaatggtatgatggacccagatgacttccgtgcctgtctcatctccatgggctacgatctgggtgaggtggagtttgcccgcatcatgacgctggtggattccaacaacacaggtgtggtgaccttccaggccttcatcgacttcatgacccgcgagacggccgagacagacaccgccgatcaggtcatggcctcattcaagatcctggcctctgacaagacatacatcacagtggaagaactgcgcagggagctgcccccagagcaggccgactactgcatcagccgcatgaccagttacatcggcagcggcgcacccccaggcgccctggactacatctccttctccagtgccctgtacggagagagcgactta

SEQ ID NO: 2 - protein, 895 aa, synthetic construct
  REGION 1..895: note = PREDICTED: alpha-actinin-3
MTAIETQVQYGSYMTSEQVYMTQEDDWDRDLLLDPAWEKQQRKTFTAWCNSHLRKAGTQIENIEEDFRNGLKLMLLLEVISGERLPKPDKGKMRFHKIANVNKALDFICSKGVKLVSIGAEEIVDGNVKMTLGMIWTIILRFAIQDISVEETSAKEGLLLWCQRKTAPYRNVNVQNFHISWKDGLALCALIHRHRPDLIDYSKLRKDDPMGNLNTAFEVAEKYLDIPKMLDAEDIVNTPKPDEKAIMTYVSCFYHAFAGAEQAETAANRICKVLAVNQENEKLMEEYEKLASELLEWIRRTIPWLENRVAEQTMRAMQQKLEDFRDYRRVHKPPKVQEKCQLEINFNTLQTKLRLSNRPAFMPSEGKMVSDIANAWKGLEQVEKGYEEWLLTEIRRLERLDHLAEKFKQKSSLHESWTSGKVELLSMKDYESASLMEIRALMRKHEAFESDLAAHQDRVEQIAAIAQELNELDYHDAVTINARCQGICDQWDNLGTLTQKRRDSLERVEKLWETIDQLYLEFAKRAAPFNNWMDGAMEDLQDMFIVHSIEEIQSLITAHDQFKATLPEADKERVATMGIQNEIVKIAQTYGIKLSGVNPYTNLSPQDITDKWDAVKHLVPLRDQMLQEEVARQQSNERLRRQFAAQANIIGPWIQTKMEEISHVSVDIAGSLEEQMSNLKQYEQNIINYKCNIDKLEGDHQLSQESLIFDNKHTNYTMEHVRVGWEQLLTTIARTINEVENQILTRDAKGISQEQLNEFRASFNHFDRKRNGMMDPDDFRACLISMGYDLGEVEFARIMTLVDSNNTGVVTFQAFIDFMTRETAETDTADQVMASFKILASDKTYITVEELRRELPPEQADYCISRMTSYIGSGAPPGALDYISFSSALYGESDL

SEQ ID NO: 3 - protein, 14 aa, synthetic construct
  REGION 1..14: note = PREDICTED: alpha-actinin-3 fragment
ERVATMGIQNEIVK

SEQ ID NO: 4 - DNA, 3591 bp, synthetic construct
  misc_feature 1..3591: note = EEF1A2 binding protein-like
  source 1..3591: mol_type = other DNA
atgtggaaaaagaaatcaaagctcacggaccagacggccactggccaagttgggatcaagaagagatcaaaagtccctggagttatgatcacgcagtatgtggagaaaataccagatgggaaaagccaccctgacttcacccgcaagcctatcgcgttgaccattcaggagggtaaattcgccttcttcaaagccctggttattggagatccagaaccaaccgtgacatggggcagaaataatggagatgtgtcagatacatcaaaatatgtgacaaaatatgaccctgctacacgtgagcacttatttgagatggccaatgtaaaaccagaacaagcagacacctacaaatgctttgcagctaatgagtttggaagagcagtggtcacagtggtcctcaatgttattgaagttgggttcaagaaagcacaagcggactcacaggtgcaaccagaggcagctgttgcagattttaaatctgtattgaagagaaaaagtaaaattcagcccaaaatggaaaagaaagaagatggagaaatagatccaagattttgggaactcttgataagtgctgacaagaaagactatgagagcctcatgttggagtttggagtcactgacttccgctttatgctgaagacactgaatgagattaagaaggaaagagaggaagagcaagcacagttcattgaaaacctagctaacctgaaacctattgaagttggacccgatggctgtgcaaccttttcaatagacatggatctcattgaacaaagcagcaggatcttcctctacaaggatggagtgatgattccatacagcaaggagttgggagatacaatcaaacacagcctaaagatagtgggccgaaagtatcagttcagcataagggatctgtttcctgatgatgctgggctctaccaggtggatgttgaggacgtaaatgtattctccaccgattttaagattcccatggtggacttcctggtcaagattcaggagtgtaaggccatggagagagaggatgctgtgtttgagtgtgtcctgtcacagcccttcggcaagatcatgtgggttggcaagaacttaccattggaggcaggggataaatatgatattgaggtttcagaagacaagctcatccacagactaatcattaaagacgttgctatggtggacaaaggcatctatgccgctgtggcaggaatcaaatcttgcaatgcctttcttgtggttgaagccgacaagggtgaacccggcaaaaagaaacaacgtaaaaccacaagggcaggaggagctggagttgacctgacggcgattgcccaagagcaggcagttaaaaacacggcagacagagaggtgctgaaggagaaggtgaaagcaatcaatgacgagagagcggctaatgccactgcagcacctgagacttcagctgaagctaaagctaaagttaaggtggtagaggcttcccaaacaggagctccaaaacagggaccggcagttaaagggtctgaccataaatccgtggataatgagggatacagcattaagagtggactctcagatgtctttgctctccgtggcaagaaaggtgaactggtttgtgagatgagccatgaagttgatggggcctggttcaaggatggagagaagttatccaccacagatggaatagccatagtgaaagatggaacgagacacacgatgaccattcatagcagtagtgaagacgacactggagtctatcacattgaagcggggggatttaaatcagaggcaaaagtcactgtgggagaattacctgccattgatgctgatgacctccacaagttttctaagcctgtgacaataaaagtgggccagaatgcatcctggaagatgccttatacaccacaggacaacttggaggtgaaatggtttaaggatggcaaagagttgaaggatggtggtggggtgaagttggtgaaggaggtcaagcacagccggctgctgctccgggagtgtctgcgttctgacgctggggagatcaagattcaactcaaaaacccctttggcactatagaggccacatctcgactgattgtccttgacaagcccggcccaccagaaggcccggtggaaatcttggagaccacctccactgtgattgagctgcagtggggcgctcctaaggacgacggtggctccccggtgactaactacatcattgagcgccagcagctgggacagaccgtgtggaagaagatgggtgatgtcgcagctgacaaaaccacctacagggacaggaatgtggtccatgggaaactgtacatctacaatatctacgcagtgaacccagaggggaccagtgatgcactgcagactgaggaaacaatggctggcgtattgatatttgctggccgacctggagcaccaaaagtggtcagcgcatcaaagacctgcatcaacctgaaatgggagcccccagaagatgacggaggaattaagattgatggctatcaactggaaaaacgcaaaaaggacacagctcagtggattgctttgaacccagtaactgagcctattgaagtgctggagtacgcagtgaaggatgttgttgagggggcggagtatgagttcagggtatcggccatcaacgtttctggggcgggagagttcagtctcccctctgtgatggtgactgcaaagaatcccaacatgaggcctacattcaaagatccagaggacttcatggtgatcagggcgggaaactctgtgagaatcaaagttctctatgaggctgagcctccaccggagatcacttggatgaaggacaatgagcctgtatccagttttatacagatcatcaacacagagggctgttcccagcttgtgatcccctcaacaaaacgctctgattcaggaaattacaccattgtggctaagaataaagttggagaggccagctttgacattgaggtcctagtcacagatgagccaaagcctcctggtgcagtggagctggagcagattgtccatggcaaagttattgtgtcctgggaggcctctccagaccaggagctggacaacaggctgtattacatggtggccgagcacgactccagcacacgcatgtggcacactgtggcagaccgcatctttgacaattcatacacagccaataacatcatgcctggaagggagtaccacttcaaaatctatgccaagaacgacatgggcatgtcagacccctccatgtcacccacctggggcatcaacagcaacagaattcctataaacacaaacacgccagtggaagtcagctttgagaaaccaccatctgtcctggtccctctgaagatgcactcgccaccaaaaggattccagatgtacatgacctgcgccatccggggatgccccacacccagcgtcacgtggcacctgaacaacgtctgcatcaatggtgacagcaactactacatcaccaactcgtatggcgtgtgctccatgtacatccttagggtcaggcccaaggacgccggagaatacaaagttgtcgcagttaactccttcggcaaggcagaatgctccactaaacttgttgttaaagac

SEQ ID NO: 5 - protein, 1197 aa, synthetic construct
  REGION 1..1197: note = EEF1A2 binding protein-like
MWKKKSKLTDQTATGQVGIKKRSKVPGVMITQYVEKIPDGKSHPDFTRKPIALTIQEGKFAFFKALVIGDPEPTVTWGRNNGDVSDTSKYVTKYDPATREHLFEMANVKPEQADTYKCFAANEFGRAVVTVVLNVIEVGFKKAQADSQVQPEAAVADFKSVLKRKSKIQPKMEKKEDGEIDPRFWELLISADKKDYESLMLEFGVTDFRFMLKTLNEIKKEREEEQAQFIENLANLKPIEVGPDGCATFSIDMDLIEQSSRIFLYKDGVMIPYSKELGDTIKHSLKIVGRKYQFSIRDLFPDDAGLYQVDVEDVNVFSTDFKIPMVDFLVKIQECKAMEREDAVFECVLSQPFGKIMWVGKNLPLEAGDKYDIEVSEDKLIHRLIIKDVAMVDKGIYAAVAGIKSCNAFLVVEADKGEPGKKKQRKTTRAGGAGVDLTAIAQEQAVKNTADREVLKEKVKAINDERAANATAAPETSAEAKAKVKVVEASQTGAPKQGPAVKGSDHKSVDNEGYSIKSGLSDVFALRGKKGELVCEMSHEVDGAWFKDGEKLSTTDGIAIVKDGTRHTMTIHSSSEDDTGVYHIEAGGFKSEAKVTVGELPAIDADDLHKFSKPVTIKVGQNASWKMPYTPQDNLEVKWFKDGKELKDGGGVKLVKEVKHSRLLLRECLRSDAGEIKIQLKNPFGTIEATSRLIVLDKPGPPEGPVEILETTSTVIELQWGAPKDDGGSPVTNYIIERQQLGQTVWKKMGDVAADKTTYRDRNVVHGKLYIYNIYAVNPEGTSDALQTEETMAGVLIFAGRPGAPKVVSASKTCINLKWEPPEDDGGIKIDGYQLEKRKKDTAQWIALNPVTEPIEVLEYAVKDVVEGAEYEFRVSAINVSGAGEFSLPSVMVTAKNPNMRPTFKDPEDFMVIRAGNSVRIKVLYEAEPPPEITWMKDNEPVSSFIQIINTEGCSQLVIPSTKRSDSGNYTIVAKNKVGEASFDIEVLVTDEPKPPGAVELEQIVHGKVIVSWEASPDQELDNRLYYMVAEHDSSTRMWHTVADRIFDNSYTANNIMPGREYHFKIYAKNDMGMSDPSMSPTWGINSNRIPINTNTPVEVSFEKPPSVLVPLKMHSPPKGFQMYMTCAIRGCPTPSVTWHLNNVCINGDSNYYITNSYGVCSMYILRVRPKDAGEYKVVAVNSFGKAECSTKLVVKD

SEQ ID NO: 6 - protein, 17 aa, synthetic construct
  REGION 1..17: note = EEF1A2 binding protein-like fragment
AQADSQVQPEAAVADFK

SEQ ID NO: 7 - protein, 10 aa, synthetic construct
  REGION 1..10: note = EEF1A2 binding protein-like fragment
GIYAAVAGIK

SEQ ID NO: 8 - protein, 21 aa, synthetic construct
  REGION 1..21: note = EEF1A2 binding protein-like fragment
TTRAGGAGVDLTAIAQEQAVK

SEQ ID NO: 9 - DNA, 2532 bp, synthetic construct
  misc_feature 1..2532: note = Alpha-1,4 glucan phosphorylase
  source 1..2532: mol_type = other DNA
atgtcaaaaccgttgtcagatcacgatagaaagaagcagatctccgtgagaggtcttgctggtgtggaaaatgtggcagaactgaaggtcgctttcaacaggcatctccattttacgctggtcaaggacagaaatgtggcatccaaacgggattactactttgctctcgccaacaccgtgcgtgaccacttggtgggcaggtggatcagaacccagcaatactactatgagaaagatcccaaacgtgtgtactacatctccctggagttctacatgggtcgcaccctgcagaacaccatggtgaacctggcgctggaaaacgcctgtgatgaggccatataccagctgggtctggacatggaggagctggaggacatggaggaggacgcaggcctgggaaacggtggtcttggacgtcttgccgcctgcttcctggactctatggcttctctgggtctggctgcgtatggctacggtatccgctatgagtttggcatctttaaccagaagatcgtcaatggatggcaggttgaggaggctgatgactggctgcgttacggcaacccctgggagaaggcccgacccgagtacatgcgccccgtcaagttctatggcagaaccgagcacaccccagagggtgtgaaatgggttgacactcaggtagtgttggctctgccatatgacacccctatccccgggtacagaaacaacattgtcaacaccatgagactgtggtctgcaaaggccccatgcgacttcaacctgaaagacttcaacgttggtgggtacattcaggctgtgttggacagaaacctatgcgagaacatttcccgtgtgctgtaccccaatgataacttctttgagggcaaggagctgcgtctgaagcaggagtactttgtggtggccgccacccttcaggacatcgtccgtcgtttcaaggcctctaagtttggctccagagagatcgtccgcacagacttcgcccagctgcccaacaaagttgccatccagctgaatgacactcaccctgccatggccattcctgagctgatgagggtactggttgatgaggagaagctggagtgggacaaggcctgggacgtgtgtgtccgtacctgtgcctacacaaaccacaccgtgctgcctgaggccctggagcgctggcccattgacctgttccatcacctgctgccccgtcacctggagattatctacgagatcaaccgtcgcttcctgcagtacgtcgcctcgaagttccctggcgacaacgaccgtctgcgccgcatgtccctgattgaggagggagaatgcaagaaagtcaacatggctcatatgtgtatcgttggatcccatgctgtcaacggcgtggcccgcatccactcggagatcctcgtcgccactctgttcaaggacttttatgagttggacccacacaagttccagaacaagaccaatggcatcaccccccgtcgctggctggttatgtgcaaccccggcttggctgaggtcatcgcagagagaattggagaggagtttgtccgtgaccttgaccagctgaagaaactgttgaagttcattgatgatgatgctttcatccgtgacatagccaaagtcaagcaggagaacaagctgaagttcgctgtgcacctggaagaacactacaaagtaaagatcaacccccagtccatgtttgacttccaagtcaaaagaatccacgagtacaagagacagctgctcaactgtctgcacatgatcacctactacaaccgtatcaagaaggagccaaacaagcactggaccccaagaaccatcatggtcggaggaaaggctgccccaggctaccacacagccaagatgatcattcgtctcatcaccgctatcggtgaggttgtcaaccacgaccccgtgatcggcgaccgcctcaaagtcatcttcttggagaactacagagtcaccctggctgagaaagccatcccctctgctgacctgtctgagcagatctctacagctggcactgaggcctctggcactggcaacatgaagttcatgctgaatggcgctctgaccatcggcaccatggacggagccaacgtggagatggccgaggaggccggagagaagaacctcttcatcttcggcatgagagtggaggaagtggacgcaatggacgccggcaaaggataccacgcctctgagtactacaaccgtattcccgagctgaaacaggccatggaccagatctctggtggcttcttcagccataagcagccagacctcttcaaggaacttgtggacctgctgatgcaccacgacaggttcaaggtgtttgctgactacgaagcctacatcaaaagtcaggataaggtcaacgaactgtacaagaaacccaaggaatggaccaagatggtgatccataacattgcaggctgtggtaaattctccagcgaccgcaccatttcccagtacgcccgggagatctggggcatggagcccagcctggagaagatccctgcccccgatgagcaactcaaa

SEQ ID NO: 10 - protein, 844 aa, synthetic construct
  REGION 1..844: note = Alpha-1,4 glucan phosphorylase
MSKPLSDHDRKKQISVRGLAGVENVAELKVAFNRHLHFTLVKDRNVASKRDYYFALANTVRDHLVGRWIRTQQYYYEKDPKRVYYISLEFYMGRTLQNTMVNLALENACDEAIYQLGLDMEELEDMEEDAGLGNGGLGRLAACFLDSMASLGLAAYGYGIRYEFGIFNQKIVNGWQVEEADDWLRYGNPWEKARPEYMRPVKFYGRTEHTPEGVKWVDTQVVLALPYDTPIPGYRNNIVNTMRLWSAKAPCDFNLKDFNVGGYIQAVLDRNLCENISRVLYPNDNFFEGKELRLKQEYFVVAATLQDIVRRFKASKFGSREIVRTDFAQLPNKVAIQLNDTHPAMAIPELMRVLVDEEKLEWDKAWDVCVRTCAYTNHTVLPEALERWPIDLFHHLLPRHLEIIYEINRRFLQYVASKFPGDNDRLRRMSLIEEGECKKVNMAHMCIVGSHAVNGVARIHSEILVATLFKDFYELDPHKFQNKTNGITPRRWLVMCNPGLAEVIAERIGEEFVRDLDQLKKLLKFIDDDAFIRDIAKVKQENKLKFAVHLEEHYKVKINPQSMFDFQVKRIHEYKRQLLNCLHMITYYNRIKKEPNKHWTPRTIMVGGKAAPGYHTAKMIIRLITAIGEVVNHDPVIGDRLKVIFLENYRVTLAEKAIPSADLSEQISTAGTEASGTGNMKFMLNGALTIGTMDGANVEMAEEAGEKNLFIFGMRVEEVDAMDAGKGYHASEYYNRIPELKQAMDQISGGFFSHKQPDLFKELVDLLMHHDRFKVFADYEAYIKSQDKVNELYKKPKEWTKMVIHNIAGCGKFSSDRTISQYAREIWGMEPSLEKIPAPDEQLK

SEQ ID NO: 11 - protein, 9 aa, synthetic construct
  REGION 1..9: note = Alpha-1,4 glucan phosphorylase fragment
IPAPDEQLK

SEQ ID NO: 12 - protein, 17 aa, synthetic construct
  REGION 1..17: note = Alpha-1,4 glucan phosphorylase fragment
QISVRGLAGVENVAELK

SEQ ID NO: 13 - protein, 25 aa, synthetic construct
  REGION 1..25: note = Alpha-1,4 glucan phosphorylase fragment
AIPSADLSEQISTAGTEASGTGNMK

SEQ ID NO: 14 - protein, 9 aa, synthetic construct
  REGION 1..9: note = Alpha-1,4 glucan phosphorylase fragment
DFYELDPHK

SEQ ID NO: 15 - protein, 10 aa, synthetic construct
  REGION 1..10: note = Alpha-1,4 glucan phosphorylase fragment
FAVHLEEHYK

SEQ ID NO: 16 - protein, 15 aa, synthetic construct
  REGION 1..15: note = Alpha-1,4 glucan phosphorylase fragment
GYHASEYYNRIPELK

SEQ ID NO: 17 - protein, 9 aa, synthetic construct
  REGION 1..9: note = Alpha-1,4 glucan phosphorylase fragment
IPAPDEQLK

SEQ ID NO: 18 - DNA, 2574 bp, synthetic construct
  misc_feature 1..2574: note = Elongation factor 2
  source 1..2574: mol_type = other DNA
atggtgaactttacagtggaccagatccgtgccatcatggacaagaaatccaacattcgtaacatgtctgtgatcgctcacgtggaccacggcaagtccacgctgaccgactccctggtgtctaaagccgggatcatcgcggggtctcgcgccggagagacacgcttcacagacactcgcaaagacgagcaggagcgctgtattaccatcaagtccacggctatctcgatgtactatgagctgggggaaaacgacatggccttcatcaagcagtctaaggatgggcttggcttcctcatcaacctgattgactcaccgggccatgtggacttctcctctgaggtcacagccgcccttagggtcaccgacggcgccctggtggtggttgactgcgtctcaggtgtgtgtgtgcagacagagaccgtgttgaggcaggccattgctgagcgcatcaagccagtgctgatgatgaacaagatggaccgggccctgctggagctgcagctggagcctgaggacctgttccagaccttccagcgcatcgtggagaatgtcaacgtcatcattgccacctacggagaggatgaagcgggaccaatgggtgccatcatgattgaccctgtgattggtaccgtggggtttgggtctggcctccacggctgggccttcactctaaagcagtttgctgagatgtacgtgacaaagttttctgccggcaaagacacccagctgggatcggcggagaggtgtaagaaggtggaggacatgatgaagaagctgtggggggagaggttttttgacccagccactgggaagttcagtaagtccaacctcggccctgacggtaagaagctgccccgcaccttctctcagctggtcctggaccctatcttcaaggtatttgatgccatcatgaacttcaagaaggatgagacagccaagctgatagagaagctggacatcaagctggactctgaggacaaggagaaggagggcaagcccctgttgaaggcagtgatgcgtcgctggctcccagccggagaagccctgctccagatgatcaccatccacctgccctcccccgtcacggcccagaagtaccgctgtgagctgctctacgagggaccaggagacgacgaggccgccatgggtatcaagaactgcgaccccaaggctcccctgatgatgtacatatctaagatggtgcccaccacagacaagggtcgcttctatgcctttggccgtgtgttctctggctgtgtgtccaccggtctgaaggtgcgcatcatgggaccaaacttcacccctgggaagaaggaagacctctacatcaagcccatccagaggaccattctgatgatggggcgttatgtggagcccattgaggatgtaccatgtgggaacatcgttgggctggttggagttgaccagtatctgattaagactgggaccatcaccacctttgaacaggcccacaacatgcgtgtgatgaagttcagcgtcagccctgtggtgagggtggctgtggaggccaagaaccctgctgacctgcccaagctggtggaggggctgaagcgtctggccaagtctgaccccatggtgcagtgtatcatcgaggagtctggagagcacatcatcgccggggccggagagcttcatctggagatctgtctcaaggatctggaggaggaccacgccggcattcccctgaagaaatctgatccagtggtgtcctacagggagactgtgtctgaggagtctgaagtgatgtgtctatccaagtcccctaacaagcacaaccgtctgtacatgcgggctaaacctttccctgacggcctggccgaggacatcgagaagggggacgtcagcccccgacaggagctgaaaatccgcgcccgtttcctggctgacaagtacgagtgggacgtgtcggaggcccgtaagatctggtgcttcggccctgacggtaccggtcccaacctgctgatggatgttaccaagggagtccagtacctgaatgagatcaaggacagtgttgtggctggcttccagtgggctgtcaaggagggtgtgttgtgtgaggagaacatgcgtgcagtccgcttcgacatccacgacgtgaccctgcacacagacgcaattcaccgcggtggcggacagatcatccccacggcccgcagagtgctgtatgcctgccagctcaccgcccagccacgactcatggagccggtctacttagtggagatccagtgcccagagcaggtagtgggtgggatctacggcgtgctgaacaggaagcgaggccatgtgttcgaggagtcccaggtgatgggcacgcccatgttcatcgtcaaggcctacctgcctgtcaacgagtcatttgggttcaccgctgacctgcgctccaacacgggcggccaggctttcccccagtgtgtgtttgatcactggcagatcctccagggagatccccaggaccccaccaccaagaccgccattgtggtggccgagaccaggaaacgcaaggggctgaaagagggcatcccggccctggacaactacctggacaaattg

SEQ ID NO: 19 - protein, 858 aa, synthetic construct
  REGION 1..858: note = Elongation factor 2
MVNFTVDQIRAIMDKKSNIRNMSVIAHVDHGKSTLTDSLVSKAGIIAGSRAGETRFTDTRKDEQERCITIKSTAISMYYELGENDMAFIKQSKDGLGFLINLIDSPGHVDFSSEVTAALRVTDGALVVVDCVSGVCVQTETVLRQAIAERIKPVLMMNKMDRALLELQLEPEDLFQTFQRIVENVNVIIATYGEDEAGPMGAIMIDPVIGTVGFGSGLHGWAFTLKQFAEMYVTKFSAGKDTQLGSAERCKKVEDMMKKLWGERFFDPATGKFSKSNLGPDGKKLPRTFSQLVLDPIFKVFDAIMNFKKDETAKLIEKLDIKLDSEDKEKEGKPLLKAVMRRWLPAGEALLQMITIHLPSPVTAQKYRCELLYEGPGDDEAAMGIKNCDPKAPLMMYISKMVPTTDKGRFYAFGRVFSGCVSTGLKVRIMGPNFTPGKKEDLYIKPIQRTILMMGRYVEPIEDVPCGNIVGLVGVDQYLIKTGTITTFEQAHNMRVMKFSVSPVVRVAVEAKNPADLPKLVEGLKRLAKSDPMVQCIIEESGEHIIAGAGELHLEICLKDLEEDHAGIPLKKSDPVVSYRETVSEESEVMCLSKSPNKHNRLYMRAKPFPDGLAEDIEKGDVSPRQELKIRARFLADKYEWDVSEARKIWCFGPDGTGPNLLMDVTKGVQYLNEIKDSVVAGFQWAVKEGVLCEENMRAVRFDIHDVTLHTDAIHRGGGQIIPTARRVLYACQLTAQPRLMEPVYLVEIQCPEQVVGGIYGVLNRKRGHVFEESQVMGTPMFIVKAYLPVNESFGFTADLRSNTGGQAFPQCVFDHWQILQGDPQDPTTKTAIVVAETRKRKGLKEGIPALDNYLDKL

SEQ ID NO: 20 - protein, 10 aa, synthetic construct
  REGION 1..10: note = Elongation factor 2 fragment
TAIVVAETRK

SEQ ID NO: 21 - protein, 12 aa, synthetic construct
  REGION 1..12: note = Elongation factor 2 fragment
DLEEDHAGIPLK

SEQ ID NO: 22 - protein, 13 aa, synthetic construct
  REGION 1..13: note = Elongation factor 2 fragment
DLEEDHAGIPLKK

SEQ ID NO: 23 - protein, 12 aa, synthetic construct
  REGION 1..12: note = Elongation factor 2 fragment
DSVVAGFQWAVK

SEQ ID NO: 24 - protein, 10 aa, synthetic construct
  REGION 1..10: note = Elongation factor 2 fragment
GDVSPRQELK

SEQ ID NO: 25 - DNA, 1989 bp, synthetic construct
  misc_feature 1..1989: note = Heat shock cognate 70 kDa protein
  source 1..1989: mol_type = other DNA
atgtcaaagggaccagcagttggcattgacctgggcaccacctactcctgtgtgggtgtgtttcagcatggcaaagtggagatcatagccaacgaccagggaaacaggaccacacccagttatgtcgccttcacagacacagaaaggctgattggggacgcagccaagaaccaagtggccatgaaccccacaaacacagtgtttgatgctaagcggctgatagggcggaagtttgacgacagtgtcgtccaggcagacatgaaacactggccgtttacagtgatcaacgactcgacacggcccaaggtccaagtggagtacaagggagagaccaaggccttctacccagaggagatctcctccatggtgctggtcaagatgaaggagattgcagaggcctacctgggcaagactataaccaatgcagtggtcactgtgccagcttacttcaacgactctcagcgccaggccaccaaagatgcagggactatctcaggactcaacgtactccgcatcatcaatgagccaactgctgctgctatcgcctatggcctggacaagaaggtgggagtggagagaaacgtcctaatctttgacctaggcggaggtacgtttgatgtgtctatcctgaccatcgaagacgggatctttgaggtgaagtccacggccggagacacccatcttggaggagaggacttcgacaaccgcatggtcaaccacttcatctctgagttcaagcgcaaatacaagaaggacatcagcgataacaagagggccgtgcgtcgtctgcgcaccgcctgtgaacgtgccaagcgcaccctgtcctccagtacccaggccagcattgagattgactctctgtatgagggcgtcgacttctacacctccatcaccagggctcgctttgaggagctgaacgctgacctgttcagaggcaccctggaccccgtggagaagtctctgagggacgccaagatggacaaggcccaggttcacgacatcgtcctagtgggaggctccacacgcatccccaagatccaaaagttgctgcaggacttcttcaatgggaaggagctcaacaagagcatcaaccctgatgaggcggttgcctatggtgcagctgttcaggcggccatcttgtccggagacaagtctgagaacgtgcaggacctgctgctgctggacgtcacgccactgtccctgggcattgagacggccggaggggtcatgaccgtgctcatcaagaggaacaccaccatccccaccaagcagacgcagaccttcacaacatactcagacaaccagcctggggtgctcattcaggtatatgagggggaaagagccatgaccaaagacaacaacctactggggaagtttgagttgtgtgggattccaccagccccccggggtgtgcctcagatcgaggtcacctttgacattgacgccaacggcatcatgaacgtgtctgccgctgacaagagcaccggcaaggagaataagatcaccatcaccaatgacaaaggtcgtctgagtaaggaggacatagagcgcatggtccaggaggctgaacagtacaaagctgcggacgatgtccagagggacaaggtggcgtccaagaatggcctggagtcatacgccttcaacatgaagtccaccgtggaggacgagaagctcaagggcaagctcagcgacgaggacaaacagaagatcctggacaaatgcaatgaggtcatcagctggctggacaagaaccagtctgcagagaaggaggagtttgagcaccaccagaaggagctggagaaggtctgtaaccccatcatcaccaagctgtaccagggtgctggggggatgcctggagggatgcctggagggatgcctggagggatgcctggagggatgcctggggggatgcctggagggatgcccggggctggtggtgccgcacccggaggaggtggatcctctggaccaacaattgaggaagtcgac

SEQ ID NO: 26 - protein, 663 aa, synthetic construct
  REGION 1..663: note = Heat shock cognate 70 kDa protein
MSKGPAVGIDLGTTYSCVGVFQHGKVEIIANDQGNRTTPSYVAFTDTERLIGDAAKNQVAMNPTNTVFDAKRLIGRKFDDSVVQADMKHWPFTVINDSTRPKVQVEYKGETKAFYPEEISSMVLVKMKEIAEAYLGKTITNAVVTVPAYFNDSQRQATKDAGTISGLNVLRIINEPTAAAIAYGLDKKVGVERNVLIFDLGGGTFDVSILTIEDGIFEVKSTAGDTHLGGEDFDNRMVNHFISEFKRKYKKDISDNKRAVRRLRTACERAKRTLSSSTQASIEIDSLYEGVDFYTSITRARFEELNADLFRGTLDPVEKSLRDAKMDKAQVHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELCGIPPAPRGVPQIEVTFDIDANGIMNVSAADKSTGKENKITITNDKGRLSKEDIERMVQEAEQYKAADDVQRDKVASKNGLESYAFNMKSTVEDEKLKGKLSDEDKQKILDKCNEVISWLDKNQSAEKEEFEHHQKELEKVCNPIITKLYQGAGGMPGGMPGGMPGGMPGGMPGGMPGGMPGAGGAAPGGGGSSGPTIEEVD

SEQ ID NO: 27 - protein, 14 aa, synthetic construct
  REGION 1..14: note = Heat shock cognate 70 kDa protein fragment
AFYPEEISSMVLVK

SEQ ID NO: 28 - protein, 14 aa, synthetic construct
  REGION 1..14: note = Heat shock cognate 70 kDa protein fragment
EDIERMVQEAEQYK

SEQ ID NO: 29 - protein, 14 aa, synthetic construct
  REGION 1..14: note = Heat shock cognate 70 kDa protein fragment
HWPFTVINDSTRPK

SEQ ID NO: 30 - protein, 14 aa, synthetic construct
  REGION 1..14: note = Heat shock cognate 70 kDa protein fragment
NQSAEKEEFEHHQK

SEQ ID NO: 31 - protein, 22 aa, synthetic construct
  REGION 1..22: note = Heat shock cognate 70 kDa protein fragment
TITNAVVTVPAYFNDSQRQATK

SEQ ID NO: 32 - DNA, 2061 bp, synthetic construct
  misc_feature 1..2061: note = Serotransferrin
  source 1..2061: mol_type = other DNA
atgaaactgcttctcgtctcagcgctgctggggtgcttcgctacggtgtatgctgccccagctgagggaatggttagatggtgcgtaaagtcggaaaaagagctgaagaaatgccacgatctcgcagccaatgtggcggggttttcatgcgtgaggagggacgactctcttgaatgcatccaagccatcaagagggaagaggctgatgctatcactctggatggaggagatatttacatagctggcctccacaactacaacctgcagcccatcattgcagaggactatggtgaggactctgacacctgctattatgctgtggccgtggccaaaaagggcactgaatttgggttcctcgacctccgtggcaagaagtcctgccacaccgggttgggcaaatctgcaggctggaacattcccatcggtaccctggtgactgtgggccagatccaatgggccggcatcgaggacagacctgtggagtcggcggtgagcgacttcttcaatgccagctgtgctccaggagccaatagggattctcaactgtgccagttgtgtatgggagactgctccagatctcacaacgagccctactatgactattccggggccttccagtgcctgaaggatggagcaggagaggttgccttcatcaagcacctgactgtacctgccgcagagaaggcaagctatgagttgctgtgcaaggataacaccagagctcccatcgacagctacaagacctgccacctggccagagtacccgcccacgctgtggtcagccgcaaggaccccaggctggccaatctcatctacagcaagctgatggccgtcacgaacttcaacctgttctcctccgatggttatgctgccaagaacctgatgttcaaggactccactcagaatctagtacagctgccaatgaccaccgactccttcctctacctgggagctgagtacatgagcactatacgctccctgacaaaagcgcaggccacaggtgtcacctccagggccatcaaatggtgtgccgtgggccataaggagaaggtcaagtgtgacgcctggacaatcaacagcttcacagatggtgactccaggatcgaatgccaggacgcacccacagtggatgaatgcatcaagaagatcatgcgtaaagaggcagatgccatagcggtggatggtggggaggtgttcactgctggaaaatgtggtctggtccctgtcatggtggagcagtatgatgaagttcggtgcagcgcccctggtgaggcgtcatcctactttgcggtggcggtggcaaagaggggatctgggttgacctggacaaccctgaagggcaagaggtcatgccacaccggcttgggcaggaccgcaggctggaacatacccatgggtcttatccataggaggactatgaactgcgacttcaccacatacttcagtaagggctgtgctcctggatttgaggtggactctcccttctgtgcccagtgtaagggcagtgggaagtccgtgggaggagatgggtccaagtgcaaagccagctctgaagagcagtactacggctataacggagcattcagatgtctggttgaagatgctggagatgttgccttcattaaacacactattgtaccagagatgactgatggtagtggtccagtttgggcacagaacctgatgtcctctgactttgaactactctgccaggatggtactaccaagccagtcacacatttccgtgagtgccacctggccaaggtgcccgcccatgctgtgataacacgcccagagtcccgtggagaggttgtgtccatccttctggagcagcaggccaggttcggctcaagcggcagcgattcctcatttaatatgttcaagccagattttggaaagaacttgctcttcaaggattccacaaagtgtctccaggagatcccaagcggcaccaaattccagggtttccttggggaagagtacatgatcgccatgcaatcgctcagggagtgctccaacagtacctcagatttggagaaggcatgcactttccattcctgc

SEQ ID NO: 33 - protein, 687 aa, synthetic construct
  REGION 1..687: note = Serotransferrin
MKLLLVSALLGCFATVYAAPAEGMVRWCVKSEKELKKCHDLAANVAGFSCVRRDDSLECIQAIKREEADAITLDGGDIYIAGLHNYNLQPIIAEDYGEDSDTCYYAVAVAKKGTEFGFLDLRGKKSCHTGLGKSAGWNIPIGTLVTVGQIQWAGIEDRPVESAVSDFFNASCAPGANRDSQLCQLCMGDCSRSHNEPYYDYSGAFQCLKDGAGEVAFIKHLTVPAAEKASYELLCKDNTRAPIDSYKTCHLARVPAHAVVSRKDPRLANLIYSKLMAVTNFNLFSSDGYAAKNLMFKDSTQNLVQLPMTTDSFLYLGAEYMSTIRSLTKAQATGVTSRAIKWCAVGHKEKVKCDAWTINSFTDGDSRIECQDAPTVDECIKKIMRKEADAIAVDGGEVFTAGKCGLVPVMVEQYDEVRCSAPGEASSYFAVAVAKRGSGLTWTTLKGKRSCHTGLGRTAGWNIPMGLIHRRTMNCDFTTYFSKGCAPGFEVDSPFCAQCKGSGKSVGGDGSKCKASSEEQYYGYNGAFRCLVEDAGDVAFIKHTIVPEMTDGSGPVWAQNLMSSDFELLCQDGTTKPVTHFRECHLAKVPAHAVITRPESRGEVVSILLEQQARFGSSGSDSSFNMFKPDFGKNLLFKDSTKCLQEIPSGTKFQGFLGEEYMIAMQSLRECSNSTSDLEKACTFHSC

SEQ ID NO: 34 - protein, 12 aa, synthetic construct
  REGION 1..12: note = Serotransferrin fragment
AQATGVTSRAIK

SEQ ID NO: 35 - protein, 10 aa, synthetic construct
  REGION 1..10: note = Serotransferrin fragment
ATGVTSRAIK

SEQ ID NO: 36 - protein, 11 aa, synthetic construct
  REGION 1..11: note = Serotransferrin fragment
DPRLANLIYSK

SEQ ID NO: 37 - protein, 12 aa, synthetic construct
  REGION 1..12: note = Serotransferrin fragment
GTEFGFLDLRGK

SEQ ID NO: 38 - protein, 13 aa, synthetic construct
  REGION 1..13: note = Serotransferrin fragment
KGTEFGFLDLRGK

SEQ ID NO: 39 - protein, 14 aa, synthetic construct
  REGION 1..14: note = Serotransferrin fragment
KGTEFGFLDLRGKK

SEQ ID NO: 40 - protein, 18 aa, synthetic construct
  REGION 1..18: note = Serotransferrin fragment
LMAVTNFNLFSSDGYAAK

SEQ ID NO: 41 - protein, 11 aa, synthetic construct
  REGION 1..11: note = Serotransferrin fragment
RGSGLTWTTLK

SEQ ID NO: 42 - DNA, 1518 bp, synthetic construct
  misc_feature 1..1518: note = Myosin binding protein H-like
  source 1..1518: mol_type = other DNA
atgcctgcgaagcctgcccctatcaagaaggcagccccgaaagagcctgctcctaagaaggaggaagcacctgcacctgcacccgcggaggcggctcctgctgaagttgctccccctgccgaggtcgaagctccaccggcagaggctgcccctgccgaagcagctgcccctgctgaggcagctccaaccgaagaaaaggctcccgcagaacccgcgcctgagcagcccaaagcacccacaccccctcctccagatgagcccaccagtgagcccctggaagtgacagtggacgacaagagcgacacttcagtgaccatcacctggaggcctccaaagactattccagccaactctggcctggatggatacactgtggaaatcaccaaggagggaactgaagactggaaagcagccaacgaggatctgaccgtgtcctgccgttatgtcatcaagaaccagactaccggcgaccgtctgctgatccgagtggttgctgtaaaccctggtggccgcagcccccctgctaccatcgccgaccccgttctggtcaaggaggttggggctcgccccaaagtccgcctccctcgtttcctgaggcagaggtatgtcaagaaagtcggcgagaagatcaacattgtcatcccgtactctggcaaacctaagccagtggttagctggttaaaggatggacagcccctcgactcaaagagggccaacatccgtacatctgacagagacagcatcctgttcatccgtcaagcagagagggtggactctggctcatacgaaatgtgcgtgaaggtggacgactttgaggacaaagctgctatcatcatccagattattgagttacccgggccaccagccagcattaagattgtggatgtctggggattcaacgttgctttggagtggaccgtacccaaagataatggaaacacagagatcacaggctacactgtccagaaagctgacaagaagaccggggactggttcaacattttggagcactacgccagactaaatgccaccatctcagacctcattatgggcaacacctacaccttcagggtctttgcagagaacaagtgcggactcagtgaggaatgtgccgtgaccaagggagaagccaccattgtgaaggaggttattgactacaaacctaccccgttcgtggagcatgacttcaccgaggctcccaagttcaccactgttctgaacgacaggtccaccactgttggctacagcaccaagctgctgtgttctgtgaggggatgccccaagcctaagattatgtggatgaagaacaagatgattctcaacaacatggacaaccccaagtacaggatgatcagcacagggggcatctgcaccctggagatccgtaagcctggcccatacgacggtggtgagtatgtctgcagagccgagaatacactggggaaggtcgacaccggctgcaagctggaggtcagaaagcctgggcaagcagatgctgacaaagacaagaaggaacag

SEQ ID NO: 43 - protein, 506 aa, synthetic construct
  REGION 1..506: note = Myosin binding protein H-like
MPAKPAPIKKAAPKEPAPKKEEAPAPAPAEAAPAEVAPPAEVEAPPAEAAPAEAAAPAEAAPTEEKAPAEPAPEQPKAPTPPPPDEPTSEPLEVTVDDKSDTSVTITWRPPKTIPANSGLDGYTVEITKEGTEDWKAANEDLTVSCRYVIKNQTTGDRLLIRVVAVNPGGRSPPATIADPVLVKEVGARPKVRLPRFLRQRYVKKVGEKINIVIPYSGKPKPVVSWLKDGQPLDSKRANIRTSDRDSILFIRQAERVDSGSYEMCVKVDDFEDKAAIIIQIIELPGPPASIKIVDVWGFNVALEWTVPKDNGNTEITGYTVQKADKKTGDWFNILEHYARLNATISDLIMGNTYTFRVFAENKCGLSEECAVTKGEATIVKEVIDYKPTPFVEHDFTEAPKFTTVLNDRSTTVGYSTKLLCSVRGCPKPKIMWMKNKMILNNMDNPKYRMISTGGICTLEIRKPGPYDGGEYVCRAENTLGKVDTGCKLEVRKPGQADADKDKKEQ

SEQ ID NO: 44 - protein, 18 aa, synthetic construct
  REGION 1..18: note = Myosin binding protein H-like fragment
AAIIIQIIELPGPPASIK

SEQ ID NO: 45 - protein, 18 aa, synthetic construct
  REGION 1..18: note = Myosin binding protein H-like fragment
AAPAEAAAPAEAAPTEEK

SEQ ID NO: 46 - protein, 11 aa, synthetic construct
  REGION 1..11: note = Myosin binding protein H-like fragment
APAEPAPEQPK

SEQ ID NO: 47 - protein, 14 aa, synthetic construct
  REGION 1..14: note = Myosin binding protein H-like fragment
DNGNTEITGYTVQK

SEQ ID NO: 48 - protein, 20 aa, synthetic construct
  REGION 1..20: note = Myosin binding protein H-like fragment
EVIDYKPTPFVEHDFTEAPK

SEQ ID NO: 49 - protein, 17 aa, synthetic construct
  REGION 1..17: note = Myosin binding protein H-like fragment
FTTVLNDRSTTVGYSTK

SEQ ID NO: 50 - protein, 10 aa, synthetic construct
  REGION 1..10: note = Myosin binding protein H-like fragment
INIVIPYSGK

SEQ ID NO: 51 - protein, 19 aa, synthetic construct
  REGION 1..19: note = Myosin binding protein H-like fragment
INIVIPYSGKPKPVVSWLK

SEQ ID NO: 52 - protein, 33 aa, synthetic construct
  REGION 1..33: note = Myosin binding protein H-like fragment
NQTTGDRLLIRVVAVNPGGRSPPATIADPVLVK

SEQ ID NO: 53 - protein, 13 aa, synthetic construct
  REGION 1..13: note = Myosin binding protein H-like fragment
SDTSVTITWRPPK

SEQ ID NO: 54 - protein, 17 aa, synthetic construct
  REGION 1..17: note = Myosin binding protein H-like fragment
TIPANSGLDGYTVEITK

SEQ ID NO: 55 - DNA, 1377 bp, synthetic construct
  misc_feature 1..1377: note = Desmin (fragment)
  source 1..1377: mol_type = other DNA
atttcctcttaccgccgcactttcggttctggtattggatccaccccaggcatgtcctccatgttctcccacggtggacgcggctcctccggctcggcccacatgtcctccagagtctacgagatgaccaaaagctccgcccgccccagctactcctccggcagtattcgctcctcctccggcggcgcgatgcgctcgtacgccgggatgggcgagaagctggacttcaacctggccgacgccactaaccgcgacttcctggacaccagaaccaatgagaaggcagagttgcagcacctgaacgaccggttcgccagctacatcgagaaggtccgtttcctggagcagcagaacgctactctggtggtggagatcgagaggctgaggggtcacgagccgacccgcgtggccgagatgtacgaggaggagatgagagagctgaggcgtcaggtggacggcatgtccaatgaccgagcccgcatggaggtggagagagacaacttggctgacgacctgcagaaactcaaactcagactgcaggaggtgatccaccagagggaagaggcagagaacaacctgtctgccttcagagctgacgtggactctgccacgctggccaggctggacctggagagacgcattgaaagcctgcaggaggagatcaccttcctcaagaagatccacgaggaggagatccatgagctgacgagccagatgcaggagacctcggtgcaggtccagatggacatgtccaaaccagacctgaccgttgccctcagggacatccgtatgcagtacgagggtatcgcagccaagaacatctctgaggcagaggactggtacaagtccaaggtgtcagacctgaaccaggctgtgaacaagaacaacgatgctctgagacaggccaaacaggagagcatggagttcaggcatcagatccagtcctacacctgtgagatcgactctctcaagggaaccaacgagtctctgctgaggcagatgagggagatggaggatcgtctgggaaacgaggctggaggttaccaggactccgtcacccgtctggaggctgagatcgccaagatgaaggatgagatggctcgtcacctcagagagtaccaggacctcctcaatgtcaagatggccctggatatagagatcgctacctacaggaagctactggagggagaggagagccgaatcactgtatctggctccaagtcttctcactctggctcccactctgctgcctcattatattccactgttggattcagagagaccagcccagacgtcggacgatcagcagaggtccactccaagaagactgtcctgatcaagaccatcgagacccgcgatggagaggtcgtcagcgag

SEQ ID NO: 56 - protein, 459 aa, synthetic construct
  REGION 1..459: note = Desmin (fragment)
ISSYRRTFGSGIGSTPGMSSMFSHGGRGSSGSAHMSSRVYEMTKSSARPSYSSGSIRSSSGGAMRSYAGMGEKLDFNLADATNRDFLDTRTNEKAELQHLNDRFASYIEKVRFLEQQNATLVVEIERLRGHEPTRVAEMYEEEMRELRRQVDGMSNDRARMEVERDNLADDLQKLKLRLQEVIHQREEAENNLSAFRADVDSATLARLDLERRIESLQEEITFLKKIHEEEIHELTSQMQETSVQVQMDMSKPDLTVALRDIRMQYEGIAAKNISEAEDWYKSKVSDLNQAVNKNNDALRQAKQESMEFRHQIQSYTCEIDSLKGTNESLLRQMREMEDRLGNEAGGYQDSVTRLEAEIAKMKDEMARHLREYQDLLNVKMALDIEIATYRKLLEGEESRITVSGSKSSHSGSHSAASLYSTVGFRETSPDVGRSAEVHSKKTVLIKTIETRDGEVVSE

SEQ ID NO: 57 - protein, 17 aa, synthetic construct
  REGION 1..17: note = A part of Desmin (fragment)
DEMARHLREYQDLLNVK

SEQ ID NO: 58 - protein, 21 aa, synthetic construct
  REGION 1..21: note = A part of Desmin (fragment)
LDFNLADATNRDFLDTRTNEK

SEQ ID NO: 59 - protein, 10 aa, synthetic construct
  REGION 1..10: note = A part of Desmin (fragment)
NISEAEDWYK

SEQ ID NO: 60 - DNA, 822 bp, synthetic construct
  misc_feature 1..822: note = Capping protein (Actin filament) muscle Z-line beta
  source 1..822: mol_type = other DNA
atgaatgaccagcagctggactgtgctctggacctgatgaggcgtcttcctccccagcagatcgagaagaatctcagtgacctcatcgacctggtgcccagtctgtgtgaggacctactctcctctgtggaccagcctctgaagatcgcccgggacaaggtagtggggaaagactacctgctctgtgactataacagagacggtgactcctacagatccccatggagtaataagtatgagccacccatcgacgatggtgccatgccatctgcccgcctgcgcaaactagaggtggaagccaacaatgcctttgaccagtatagagacctgtactttgagggtggcgtatcgtctgtgtatctgtgggacttggatcatggctttgctggggtcatcctcatcaagaaggctggagacggctccaagaagatcaaaggctgctgggactccatccatgtggtggaggtgcaggagaagtccagcggacggaccgctcactacaaactcacctccaccgtcatgctgtggctccagacgaccaaggccgggtctggaaccatgaacctgggtggcagtctgacaagacagatggagaaagacgagacagttggagagtcttccccacatattgccaacatcggccgcctggtggaggatatggagaataagattcgctccactctcaacgagatctactttgggaagaccaaggacatcgtcaatggtttaagatctattgactctctgcctgataaccaaaagtaccggcagctccagaaggagctgtctcaggtccttacccagcgccagatcttcattgac

SEQ ID NO: 61 - protein, 274 aa, synthetic construct
  REGION 1..274: note = Capping protein (Actin filament) muscle Z-line beta
MNDQQLDCALDLMRRLPPQQIEKNLSDLIDLVPSLCEDLLSSVDQPLKIARDKVVGKDYLLCDYNRDGDSYRSPWSNKYEPPIDDGAMPSARLRKLEVEANNAFDQYRDLYFEGGVSSVYLWDLDHGFAGVILIKKAGDGSKKIKGCWDSIHVVEVQEKSSGRTAHYKLTSTVMLWLQTTKAGSGTMNLGGSLTRQMEKDETVGESSPHIANIGRLVEDMENKIRSTLNEIYFGKTKDIVNGLRSIDSLPDNQKYRQLQKELSQVLTQRQIFID

SEQ ID NO: 62 - protein, 24 aa, synthetic construct
  REGION 1..24: note = Capping protein (Actin filament) muscle Z-line beta fragment
DETVGESSPHIANIGRLVEDMENK

SEQ ID NO: 63 - protein, 17 aa, synthetic construct
  REGION 1..17: note = Capping protein (Actin filament) muscle Z-line beta fragment
DIVNGLRSIDSLPDNQK

SEQ ID NO: 64 - protein, 14 aa, synthetic construct
  REGION 1..14: note = Capping protein (Actin filament) muscle Z-line beta fragment
ELSQVLTQRQIFID

SEQ ID NO: 65 - protein, 12 aa, synthetic construct
  REGION 1..12: note = Capping protein (Actin filament) muscle Z-line beta fragment
IRSTLNEIYFGK

SEQ ID NO: 66 - protein, 13 aa, synthetic construct
  REGION 1..13: note = Capping protein (Actin filament) muscle Z-line beta fragment
LTSTVMLWLQTTK

SEQ ID NO: 67 - protein, 10 aa, synthetic construct
  REGION 1..10: note = Capping protein (Actin filament) muscle Z-line beta fragment
SIDSLPDNQK

SEQ ID NO: 68 - protein, 17 aa, synthetic construct
  REGION 1..17: note = Capping protein (Actin filament) muscle Z-line beta fragment
YEPPIDDGAMPSARLRK

SEQ ID NO: 69 - DNA, 5799 bp, synthetic construct
  misc_feature 1..5799: note = myosin heavy chain, fast skeletal muscle-like
  source 1..5799: mol_type = other DNA
atgagtacggacgcggagatgcaagtctacggcaaggctgccatataccttcgtaagtctgagaaggagaggatggaggcacaagccatgccctttgattcaaagaacgcctgctatgtgacagacaaggtggagctgtaccttaagggtttagtcactgccagggccgacgggaagtgtactgtgacagctttagactttcgtgaaggaaaagagttcaaagatgcagacatctatgagatgaacccccctaagtacgacaagattgaggacatggccatgatgacctacctgaatgaagcctctgtgttgtataacctcaaagagcgttatgcagcatggatgatctatacctactctgggctcttctgtgccacggtgaacccctacaagtggctcccagtgtacgacgaagaggttgtcaacgcctacagagggaagaagagggtggaggctccaccacatatcttctccgtctctgacaacgcctttcagttcatgatgattgataaggagaaccagtccgtcctgattactggagaatccggtgcaggaaagactgtcaacaccaagcgtgtcatccagtactttgccaccattgcagtgtctggtggcaagaaggaagcagaccccaacaaaatgcaggggtctcttgaggatcagatcattgcagctaaccctctgctagagtcttacggtaatgccaagacagtgaggaacgacaactcgtctcgctttggtaaattcatcaggattcacttccaagctggtaaactggctaaagctgacattgagacctacctgctggagaagtccagagtgtccttccaactgcccgatgagagaggctaccacatcttcttccagatgatgacaggccacaaacctgagctagttgaattggcactcctcaccaccaacccctacgacttccccatgtgcagccagggtcagattgctgtggccagcatcaacgacaatgaagagctggatgccacagatgaagccattacaatcctgggcttcactaatgaggagaagcttggaatatacaagctgacaggagctgtagtgcaccatggcaacttgaaattcaagcagaagcagcgtgaggagcaggccgagccagacggcacagaggtggctgataaaatcgcctacctgctgggcctgaactcagctgagatgttgaaagctctgtgctaccccagagtgaaggtcggcaacgagtatgtgaccaagggacagactgtggctcaggttaataactcagtcagtgctctggccaagtccatctatgagaggatgttcttgtggatggtcatccgtatcaatgagatgttggacaccaagaatccaaggcagttctatatcggtgtgcttgacattgccgggtttgagatctttgattacaacagcatggagcagctgtgcatcaacttcaccaatgagaaactgcaacagtttttcaaccacaccatgttcgtcctggagcaagaggagtacaagaaggagggaatcgtctgggccttcattgacttcggcatggatttggctgcctgcattgagcttattgagaagccattgggcatcttctccatccttgaagaggagtgcatgttccccaagtcttcagacactaccttcaaggacaagctgtacgcccagcatcttggtaaaacaaaggcgtttgagaagcccaagcctgccaaaggcaaggcagaggcccacttctccctggtgcattacgccggtactgtggactacaacatcactggctggctggagaagaacaaggaccccttgaacgactcagtttgtcaactgtatgggaagtccggagtcaaaattttggctgccctgtatccccctccccctcctgaggataaagccaagaaaggaggcaagaagaagggtggttccatgcagactgtgtcctcccagttcagggagaacttacataagctgatgaccaacttgaggagcactcatcctcactttgtgcgctgcctgatccccaacgagtcaaagactccaggtctgatggagaacttcctggttatccaccagctcaggtgtaatggtgtactggagggtatcaggatctgcagaaagggcttccccagcagaatcatctatgctgacttcaagcaaaggtacaaagtactgaatgccagcgtcatccctgagggccagttcatggacaacaagaaggcttctgagaagctgcttggatccattgatgtgaatcacgaggattacaagtttggacacaccaaggtgttcttcaaagccggtctgctgggtgtcctggaggagatgagagatgagaagctggcctctctagtcggcatgctccaggctctcagccgtggattcctcatgaggagagagttcaccaagatgatggagaggagagaatcaatttactccatccagtacaacatccgctcattcatgaatgtgaaaacctggccatggatgaagttgtacttcaagatcaagcccctgctgcagagcgctgagactgagaaggagctggccaacatgaaggagaactatgagaaaatgaagacagacctggccaaggctctgtctacaaagaagcaaatggaggagaagttggtgtccctgacgcaggagaagaacgacctggcactccaagtcgcatctgaaggagagagtctgaacgatgctgaggaaaggtgcgaggggctcatcaagagcaagatccagcaggaggccaaactcaaagagacgaccgagaggctggaggatgaggaggagatcaatgctgagttgactgccaagaagaggaagctggaggatgagtgctctgagctgaagaaggacattgatgatctggagctcaccctggccaaagtggagaaggagaagcacgccactgaaaacaaggttaaaaacctgacagaggagatggcgtctatggatgagagtgttgccaagctgaccaaggagaagaaagccctacaagaggcccaccagcagacactggatgacctgcaggcagaggaggacaaagtcaacactctgaccaaggccaagaccaagctggaacagcaagtggacgaccttgagggttctctggagcaagagaagaagctccgcatggaccttgagagatccaagagaaagctggagggagatctgaaactggcccaggagtccataatggacctggagaatgacaagcagcaagctgatgagaaaatcaagaagaaggagtttgagaccactcagctcctcagcaaggttgaggatgagcagtctctgggagctcagctgcagaagaagatcaaggaactccaggcccgtattgaggaactggaggaggaaattgaggctgagcgtgctgccagggctaaggttgagaagcagagggctgatctctccagggaacttgaggagatcagcgagaggctggaggaggccggaggcgccactgctgctcagattgagatgaacaagaagcgtgaggctgagttccagaagctgcgtcgtgatcttgaagagtccaccctgcagcatgaggccacagccgccgctctgcgcaagaagcaggccgacagtgtggctgagctcggggagcagatcgacaacctgcagcgcgtcaagcagaagctggagaaggagaagagcgagtacaagatggagattgatgacctctccagcaacatggaggccgttgccaaggctaagggcaatctagagaagatgtgccgtactcttgaggaccagctgagcgagctcaagactaagaatgatgagaatgttcgccaggtcaacgacatcagcggacagagggccagactcctgacagaaaatggtgagtttggtaggcagctggaggagaaggaagccctggtgtctcagctgaccagaggcaaacaggccttcacccagcaggtggaggagctgaagagggcgattgaggaggaggtcaaggctaaaaatgcactggcccacggagttcagtctgcccgccatgactgtgacctcctgagggagcagtttgaggaggagcaggaggccaaggcagagctgcaacgcggcatgtccaaggccaatagtgaggtggctcagtggaggactaagtatgaaactgatgccatccagcgcacagaggagctggaggaggccaagaagaagctggcccagcgtctgcaggatgccgaggagaccattgaggcgaccaactccaagtgcgcctccctggagaagaccaagcagagactacagggagaggtggaggacctcatgattgatgttgagagagccaacgcattggccgccaacctcgacaagaagcagaggaactttgacaaggttctggcagagtggaagcagaagtatgaggagggtcaggctgagctggaaggagctcagaaggaggctcgctctatgagcactgagctcttcaagatgaagaactcctacgaggaggctctggatcatctggagactctgaagagagagaacaagaacctgcaacaggagatctctgaccttactgagcagattggagagactggcaagagcatccatgagctggagaaggccaagaagaccgtggagacagagaagtctgagatccagaccgctctggaggaggctgagggcacactggagcacgaggaatccaagattctgcgtgtgcagctggagctgaaccagatcaagggtgaggtggacaggaagatcgctgagaaggacgaggagatggagcagatcaagaggaacagccagaggatggttgactccatgcagagcaccctggactctgaggtcaggagcaggaatgatgccctgagggtgaagaagaagatggagggagacctgaacgagatggagatccagctgagccactccaacaggcaggccgctgaggcccagaaacagctgaggaatgtccagggacagctcaaggatgcccaattgcaccttgatgatgccgtccgtgtcgcagaggacatgaaggagcaggcagccatggtggagcgcagaaacggtctgatggtggctgagatcgaggagctgagagttgctctggagcagacagagagaggccgcaaagtggctgagactgagctggtagacgccagcgagcgtgttggactgctgcactcccagaacaccagccttctgaacaccaagaagaagctggagactgacctggtgcaggtgcagggagaggtggatgacatcgtccaggaggccaggaatgcagaagagaaggccaagaaggcaatcactgatgcggcaatgatggctgaggagctgaagaaggagcaggacaccagctctcacctggagagaatgaagaagaacctggagatcacagtcaaggacctgcagcaccgcctggatgaggctgagaatctggccatgaagggaggcaagaaacaactccagaaactggaatccagggtgcgtgagcttgagactgaggtggaggctgagcagagaagaggtgtagacgcggtaaagggagtccgcaagtatgagcgcagagtcaaggagctcacttaccagactgaggaggataagaagaatgttaacagacttcaggacctggtagataagctgcagatgaaagtgaaggcctacaagaggcacgctgaggaagcggaggaagcagcaaaccagcacatgtctaagttcaggaaggttcagcatgagctggaggaggctgaggagcgtgctgacatcgctgagactcaggtcaacaagctcagagccaagacccgtgactctggaaagggaaaagaagttgctgaa

SEQ ID NO: 70 - protein, 1933 aa, synthetic construct
  REGION 1..1933: note = myosin heavy chain, fast skeletal muscle-like
MSTDAEMQVYGKAAIYLRKSEKERMEAQAMPFDSKNACYVTDKVELYLKGLVTARADGKCTVTALDFREGKEFKDADIYEMNPPKYDKIEDMAMMTYLNEASVLYNLKERYAAWMIYTYSGLFCATVNPYKWLPVYDEEVVNAYRGKKRVEAPPHIFSVSDNAFQFMMIDKENQSVLITGESGAGKTVNTKRVIQYFATIAVSGGKKEADPNKMQGSLEDQIIAANPLLESYGNAKTVRNDNSSRFGKFIRIHFQAGKLAKADIETYLLEKSRVSFQLPDERGYHIFFQMMTGHKPELVELALLTTNPYDFPMCSQGQIAVASINDNEELDATDEAITILGFTNEEKLGIYKLTGAVVHHGNLKFKQKQREEQAEPDGTEVADKIAYLLGLNSAEMLKALCYPRVKVGNEYVTKGQTVAQVNNSVSALAKSIYERMFLWMVIRINEMLDTKNPRQFYIGVLDIAGFEIFDYNSMEQLCINFTNEKLQQFFNHTMFVLEQEEYKKEGIVWAFIDFGMDLAACIELIEKPLGIFSILEEECMFPKSSDTTFKDKLYAQHLGKTKAFEKPKPAKGKAEAHFSLVHYAGTVDYNITGWLEKNKDPLNDSVCQLYGKSGVKILAALYPPPPPEDKAKKGGKKKGGSMQTVSSQFRENLHKLMTNLRSTHPHFVRCLIPNESKTPGLMENFLVIHQLRCNGVLEGIRICRKGFPSRIIYADFKQRYKVLNASVIPEGQFMDNKKASEKLLGSIDVNHEDYKFGHTKVFFKAGLLGVLEEMRDEKLASLVGMLQALSRGFLMRREFTKMMERRESIYSIQYNIRSFMNVKTWPWMKLYFKIKPLLQSAETEKELANMKENYEKMKTDLAKALSTKKQMEEKLVSLTQEKNDLALQVASEGESLNDAEERCEGLIKSKIQQEAKLKETTERLEDEEEINAELTAKKRKLEDECSELKKDIDDLELTLAKVEKEKHATENKVKNLTEEMASMDESVAKLTKEKKALQEAHQQTLDDLQAEEDKVNTLTKAKTKLEQQVDDLEGSLEQEKKLRMDLERSKRKLEGDLKLAQESIMDLENDKQQADEKIKKKEFETTQLLSKVEDEQSLGAQLQKKIKELQARIEELEEEIEAERAARAKVEKQRADLSRELEEISERLEEAGGATAAQIEMNKKREAEFQKLRRDLEESTLQHEATAAALRKKQADSVAELGEQIDNLQRVKQKLEKEKSEYKMEIDDLSSNMEAVAKAKGNLEKMCRTLEDQLSELKTKNDENVRQVNDISGQRARLLTENGEFGRQLEEKEALVSQLTRGKQAFTQQVEELKRAIEEEVKAKNALAHGVQSARHDCDLLREQFEEEQEAKAELQRGMSKANSEVAQWRTKYETDAIQRTEELEEAKKKLAQRLQDAEETIEATNSKCASLEKTKQRLQGEVEDLMIDVERANALAANLDKKQRNFDKVLAEWKQKYEEGQAELEGAQKEARSMSTELFKMKNSYEEALDHLETLKRENKNLQQEISDLTEQIGETGKSIHELEKAKKTVETEKSEIQTALEEAEGTLEHEESKILRVQLELNQIKGEVDRKIAEKDEEMEQIKRNSQRMVDSMQSTLDSEVRSRNDALRVKKKMEGDLNEMEIQLSHSNRQAAEAQKQLRNVQGQLKDAQLHLDDAVRVAEDMKEQAAMVERRNGLMVAEIEELRVALEQTERGRKVAETELVDASERVGLLHSQNTSLLNTKKKLETDLVQVQGEVDDIVQEARNAEEKAKKAITDAAMMAEELKKEQDTSSHLERMKKNLEITVKDLQHRLDEAENLAMKGGKKQLQKLESRVRELETEVEAEQRRGVDAVKGVRKYERRVKELTYQTEEDKKNVNRLQDLVDKLQMKVKAYKRHAEEAEEAANQHMSKFRKVQHELEEAEERADIAETQVNKLRAKTRDSGKGKEVAE

SEQ ID NO: 71 - protein, 7 aa, synthetic construct
  REGION 1..7: note = myosin heavy chain, fast skeletal muscle-like fragment
AAIYLRK

SEQ ID NO: 72 - protein, 10 aa, synthetic construct
  REGION 1..10: note = myosin heavy chain, fast skeletal muscle-like fragment
ADIETYLLEK

SEQ ID NO: 73 - protein, 19 aa, synthetic construct
  REGION 1..19: note = myosin heavy chain, fast skeletal muscle-like fragment
ALQEAHQQTLDDLQAEEDK

SEQ ID NO: 74 - protein, 11 aa, synthetic construct
  REGION 1..11: note = myosin heavy chain, fast skeletal muscle-like fragment
DIDDLELTLAK

SEQ ID NO: 75 - protein, 11 aa, synthetic construct
  REGION 1..11: note = myosin heavy chain, fast skeletal muscle-like fragment
EALVSQLTRGK

SEQ ID NO: 76 - protein, 10 aa, synthetic construct
  REGION 1..10: note = myosin heavy chain, fast skeletal muscle-like fragment
EFETTQLLSK

SEQ ID NO: 77 - protein, 10 aa, synthetic construct
  REGION 1..10: note = myosin heavy chain, fast skeletal muscle-like fragment
ELTYQTEEDK

SEQ ID NO: 78 - protein, 15 aa, synthetic construct
  REGION 1..15: note = myosin heavy chain, fast skeletal muscle-like fragment
ENQSVLITGESGAGK

SEQ ID NO: 79 - protein, 19 aa, synthetic construct
  REGION 1..19: note = myosin heavy chain, fast skeletal muscle-like fragment
ETTERLEDEEEINAELTAK

SEQ ID NO: 80 - protein, 10 aa, synthetic construct
  REGION 1..10: note = myosin heavy chain, fast skeletal muscle-like fragment
FIRIHFQAGK

SEQ ID NO: 81 - protein, 12 aa, synthetic construct
  REGION 1..12: note = myosin heavy chain, fast skeletal muscle-like fragment
GFPSRIIYADFK

SEQ ID NO: 82 - protein, 10 aa, synthetic construct
  REGION 1..10: note = myosin heavy chain, fast skeletal muscle-like fragment
GLVTARADGK

SEQ ID NO: 83 - protein, 16 aa, synthetic construct
  REGION 1..16: note = myosin heavy chain, fast skeletal muscle-like fragment
GQTVAQVNNSVSALAK

SEQ ID NO: 84 - protein, 12 aa, synthetic construct
  REGION 1..12: note = myosin heavy chain, fast skeletal muscle-like fragment
IKPLLQSAETEK

SEQ ID NO: 85 - protein, 14 aa, synthetic construct
  REGION 1..14: note = myosin heavy chain, fast skeletal muscle-like fragment
ILAALYPPPPPEDK

SEQ ID NO: 86 - protein, 12 aa, synthetic construct
  REGION 1..12: note = myosin heavy chain, fast skeletal muscle-like fragment
ILRVQLELNQIK

SEQ ID NO: 87 - protein, 18 aa, synthetic construct
  REGION 1..18: note = myosin heavy chain, fast skeletal muscle-like fragment
LAQRLQDAEETIEATNSK

SEQ ID NO: 88 - protein, 16 aa, synthetic construct
  REGION 1..16: note = myosin heavy chain, fast skeletal muscle-like fragment
LEQQVDDLEGSLEQEK

SEQ ID NO: 89 - protein, 24 aa, synthetic construct
  REGION 1..24: note = myosin heavy chain, fast skeletal muscle-like fragment
LESRVRELETEVEAEQRRGVDAVK

SEQ ID NO: 90 - protein, 13 aa, synthetic construct
  REGION 1..13: note = myosin heavy chain, fast skeletal muscle-like fragment
LLGSIDVNHEDYK

SEQ ID NO: 91 - protein, 21 aa, synthetic construct
  REGION 1..21: note = myosin heavy chain, fast skeletal muscle-like fragment
LRRDLEESTLQHEATAAALRK

SEQ ID NO: 92 - protein, 12 aa, synthetic construct
  REGION 1..12: note = myosin heavy chain, fast skeletal muscle-like fragment
LTGAVVHHGNLK

SEQ ID NO: 93 - protein, 32 aa, synthetic construct
  REGION 1..32: note = myosin heavy chain, fast skeletal muscle-like fragment
NDENVRQVNDISGQRARLLTENGEFGRQLEEK

SEQ ID NO: 94 - protein, 7 aa, synthetic construct
  REGION 1..7: note = myosin heavy chain, fast skeletal muscle-like fragment
NLEITVK

SEQ ID NO: 95 - protein, 18 aa, synthetic construct
  REGION 1..18: note = myosin heavy chain, fast skeletal muscle-like fragment
NLQQEISDLTEQIGETGK

SEQ ID NO: 96 - protein, 14 aa, synthetic construct
  REGION 1..14: note = myosin heavy chain, fast skeletal muscle-like fragment
NSYEEALDHLETLK

SEQ ID NO: 97 - protein, 19 aa, synthetic construct
  REGION 1..19: note = myosin heavy chain, fast skeletal muscle-like fragment
QADSVAELGEQIDNLQRVK

SEQ ID NO: 98 - protein, 11 aa, synthetic construct
  REGION 1..11: note = myosin heavy chain, fast skeletal muscle-like fragment
QAFTQQVEELK

SEQ ID NO: 99 - protein, 16 aa, synthetic construct
  REGION 1..16: note = myosin heavy chain, fast skeletal muscle-like fragment
QREEQAEPDGTEVADK

SEQ ID NO: 100 - protein, 8 aa, synthetic construct
  REGION 1..8: note = myosin heavy chain, fast skeletal muscle-like fragment
RAIEEEVK

SEQ ID NO: 101 - protein, 20 aa, synthetic construct
  REGION 1..20: note = myosin heavy chain, fast skeletal muscle-like fragment
SEIQTALEEAEGTLEHEESK

SEQ ID NO: 102 - protein, 12 aa, synthetic construct
  REGION 1..12: note = myosin heavy chain, fast skeletal muscle-like fragment
TVRNDNSSRFGK

SEQ ID NO: 103 - protein, 27 aa, synthetic construct
  REGION 1..27: note = myosin heavy chain, fast skeletal muscle-like fragment
VAETELVDASERVGLLHSQNTSLLNTK

SEQ ID NO: 104 - protein, 13 aa, synthetic construct
  REGION 1..13: note = myosin heavy chain, fast skeletal muscle-like fragment
VEDEQSLGAQLQK

SEQ ID NO: 105 - protein, 8 aa, synthetic construct
  REGION 1..8: note = myosin heavy chain, fast skeletal muscle-like fragment
VGNEYVTK

SEQ ID NO: 106 - protein, 21 aa, synthetic construct
  REGION 1..21: note = myosin heavy chain, fast skeletal muscle-like fragment
VQHELEEAEERADIAETQVNK

SEQ ID NO: 107 - protein, 13 aa, synthetic construct
  REGION 1..13: note = myosin heavy chain, fast skeletal muscle-like fragment
YEEGQAELEGAQK

SEQ ID NO: 108 - protein, 16 aa, synthetic construct
  REGION 1..16: note = myosin heavy chain, fast skeletal muscle-like fragment
YETDAIQRTEELEEAK

SEQ ID NO: 109 - DNA, 2532 bp, synthetic construct
  misc_feature 1..2532: note = glycogen phosphorylase, muscle form-like
  source 1..2532: mol_type = other DNA
atgtcaaaaccattgtcagaccacgataaaaggaagcagatctccgtgagaggtcttgctggtgtggaaaatgtcgcagaactgaaggtcgctttcaacaggcatctccattttacactggtcaaggacagaaatgtggcaaacaaacgggattactactttgctctcgccaacaccgtgcgtgaccacttggtgggcaggtggatcagaacccagcagtactactatgagaaagatcccaaacgtgtgtactacatctccctggagttttatatgggtcgcaccctgcagaacactatggtgaacctggccctggagaacgcttgtgatgaggccatataccagctgggtctggacatggaggagctggaggacatggaagaggatgctggcctgggaaacggtggtcttggccgtcttgccgcatgcttcctggactcaatggcttctctaggccttgctgcctatggctatggtatccgctatgagtttggcatcttcaatcagaagatcgtcaatggctggcaggttgaggaggccgatgattggttgcgttacggcaacccctgggagaaggcccgccctgagtacatgcgccccgtcaagttctatggcaggaccgagcacaccccagatggtgtgaaatgggttgacactcaggtagtgttggctctgccatatgacacccctattcccggctacagaaacaacattgtcaacaccatgagactgtggtctgctaaggccccatgcgacttcaacctgaaagacttcaacgttggtggctacattcaggctgtgttggacagaaacttgtgtgagaacatttcccgcgtgctgtaccccaatgataatttctttgagggcaaggagctgcgtctgaagcaggagtactttgtggtggccgccactctgcaggacgtcgtccgtcgtttcaaggcctctaagtttggctccagagagattgtccgcacagacttcgccgagctaccaaacaaagttgccatccagctgaatgacactcaccctgccatggctattcctgagctgatgagggtcctggttgatgaggagaagctgggttgggacaaggcctgggacgtgtgtgtccgtacctgtgcctacacaaaccacaccgtgctgcctgaggccctggagcgctggcccattgacctgttccatcacctgctgccacgtcacctggagatcatctacgagatcaaccgtcgcttcatggagtatgttgcctcgaagttccctggagacaacgaccgtctgcgtcgcatgtccctgattgaggagggaggatgcaagaaagtcaacatggctcatctgtgtatcgttggttcccatgctgtcaacggcgtggcccgcatccactctgagatcctcatcgccactctgttcaaggacttctatgagttggacccacacaagtttcagaacaagaccaatgggatcaccccccgtcgctggctggttatgtgcaaccccggcttggcggaggtcatcgcagagaaaattggagaggagtttatccgtgaccttgaccagcttaagcgactgttgaagttcgttaatgatgatgctttcatccgtgacatcgccaaagtcaagcaggagaacaagctgaagttcgctgtgcacctggaagagcactacaaggtcaagatcaacccccagtccatgtttgacttccaagtcaaaagaatccacgagtacaaaagacagctgctcaactgtctgcacatgatcacctactacaaccgtatcaagaaggagcccaacaagcactggaccccaagaaccatcatggtcggaggaaaggctgcaccaggctaccacacagccaagatgatcatccgtctcatcacagctatcggtgaggttgtcaaccacgaccccgttgtcggcgaccgcctcaaagttatcttcctggagaactacagagtcaccctggctgagaaagccatcccctctgccgacctgtctgagcagatctctacagctggcaccgaggcctctggcactggtaacatgaagttcatgctgaacggtgctcttaccattggcaccatggatggagccaatgtggagatggccgaggaggccggagaggaaaacttcttcatcttcggtatgagagtggaggatgtggacgcaatggacgccggcaaaggataccacgcctctgagtactacaaccgtattcccgagctgaagcaggccatggaccagattgctggcggcttcttcagtcccaagcagcctgacctcttcaaggaacttgtggacctgctgatgcaccatgacaggttcaaggtgtttgctgactacgaagcctacatcaaatgccaggacaaggtcaaccaactgtacaagaatcccaaggaatggaccaagatggtgatccataacattgcgggctgtggtaaattctccagcgaccgcaccattgcccagtacgcccgagagatctggggcatggagcccagcctggagaagatccctgcccccgatgagaaactcaaa

SEQ ID NO: 110 - protein, 844 aa, synthetic construct
  REGION 1..844: note = glycogen phosphorylase, muscle form-like
MSKPLSDHDKRKQISVRGLAGVENVAELKVAFNRHLHFTLVKDRNVANKRDYYFALANTVRDHLVGRWIRTQQYYYEKDPKRVYYISLEFYMGRTLQNTMVNLALENACDEAIYQLGLDMEELEDMEEDAGLGNGGLGRLAACFLDSMASLGLAAYGYGIRYEFGIFNQKIVNGWQVEEADDWLRYGNPWEKARPEYMRPVKFYGRTEHTPDGVKWVDTQVVLALPYDTPIPGYRNNIVNTMRLWSAKAPCDFNLKDFNVGGYIQAVLDRNLCENISRVLYPNDNFFEGKELRLKQEYFVVAATLQDVVRRFKASKFGSREIVRTDFAELPNKVAIQLNDTHPAMAIPELMRVLVDEEKLGWDKAWDVCVRTCAYTNHTVLPEALERWPIDLFHHLLPRHLEIIYEINRRFMEYVASKFPGDNDRLRRMSLIEEGGCKKVNMAHLCIVGSHAVNGVARIHSEILIATLFKDFYELDPHKFQNKTNGITPRRWLVMCNPGLAEVIAEKIGEEFIRDLDQLKRLLKFVNDDAFIRDIAKVKQENKLKFAVHLEEHYKVKINPQSMFDFQVKRIHEYKRQLLNCLHMITYYNRIKKEPNKHWTPRTIMVGGKAAPGYHTAKMIIRLITAIGEVVNHDPVVGDRLKVIFLENYRVTLAEKAIPSADLSEQISTAGTEASGTGNMKFMLNGALTIGTMDGANVEMAEEAGEENFFIFGMRVEDVDAMDAGKGYHASEYYNRIPELKQAMDQIAGGFFSPKQPDLFKELVDLLMHHDRFKVFADYEAYIKCQDKVNQLYKNPKEWTKMVIHNIAGCGKFSSDRTIAQYAREIWGMEPSLEKIPAPDEKLK

SEQ ID NO: 111 - protein, 9 aa, synthetic construct
  REGION 1..9: note = glycogen phosphorylase, muscle form-like fragment
DFYELDPHK

SEQ ID NO: 112 - protein, 10 aa, synthetic construct
  REGION 1..10: note = glycogen phosphorylase, muscle form-like fragment
FAVHLEEHYK

SEQ ID NO: 113 - protein, 13 aa, synthetic construct
  REGION 1..13: note = glycogen phosphorylase, muscle form-like fragment
FYGRTEHTPDGVK

SEQ ID NO: 114 - protein, 15 aa, synthetic construct
  REGION 1..15: note = glycogen phosphorylase, muscle form-like fragment
GYHASEYYNRIPELK

SEQ ID NO: 115 - protein, 13 aa, synthetic construct
  REGION 1..13: note = glycogen phosphorylase, muscle form-like fragment
IGEEFIRDLDQLK

SEQ ID NO: 116 - protein, 14 aa, synthetic construct
  REGION 1..14: note = glycogen phosphorylase, muscle form-like fragment
QAMDQIAGGFFSPK

SEQ ID NO: 117 - protein, 17 aa, synthetic construct
  REGION 1..17: note = glycogen phosphorylase, muscle form-like fragment
QISVRGLAGVENVAELK

SEQ ID NO: 118 - protein, 10 aa, synthetic construct
  REGION 1..10: note = glycogen phosphorylase, muscle form-like fragment
VFADYEAYIK

SEQ ID NO: 119 - protein, 14 aa, synthetic construct
  REGION 1..14: note = glycogen phosphorylase, muscle form-like fragment
VIFLENYRVTLAEK

SEQ ID NO: 120 - DNA, 3399 bp, synthetic construct
  misc_feature 1..3399: note = myosin-binding protein C, fast-type-like
  source 1..3399: mol_type = other DNA
atgcctgaagccgctgatgcagtgaagccggagggcgaggagggagaagcaccagcagaagccgaagaaaatactgcagaagatgatgagccactgcctgaaggagaggagggtgcccatcagtcacaggagattacaggactgtttcttcagcaccctgagactacggttgccataacaggtacggacattgtgtttactgccaaagtggactctactacactatcaaggaaacccaccattaaatggctgaaggggaagtggctggacctgggcagcaaagcaggaaaacacatgcagttcaaagagacttttgacagagccacaaagatctacacctgggacatgaagataatcaaggtggtccctggtgacgctggggcctacaggtgtgaggtcacctccaaagacaagtgtgacagcagcgctttcgacatctccgtggaggctgtgcaaattgaggaggaacatgattctttggccgcattcaaaagaacggatgctggagaggatgaaggcagtttggatttcagtgctttgctgaaagctaccaagaagaagaagaagccagtcgtagatgaggagaaggcagacgtgtgggaaatcctgaagaacgctcaacccagcgagtatgagaagattgcttttgagtacggcattacagacctgaggggtctgctcaagcgactgaagaagatgaagactgttgagcccaagcacagcgacgctttcctaaagagaatggagtctgcctactctgtggataagggcaagaaaatcgtactgcaggtggaagtggttgacccaaatgcccaggtcaaatggttgaagaacggtcaggagataaaatcatcagccaagtacatcatagaatcagttggcaacatcaggacgctcaccatcaacaagtgtagcctggctgacgacgctgcgtacgagtgtgtgattggggaagagaagtccttcacagaggtgtttgtcaaagagcccccggtcaccatcaccaagctgctggatgattaccacgtggtggtgggagagagagtggagtttgaggtggaggtgtctgtggagggagcacacgttaactggatgtttgaggaccaagaactctccagggacacccataagtaccgctttaagaaggacgggttgaagcacatgctcatcatccaagaggctacgctggatgacattggaatgtactggtgcttcaccaacggcgggcgaaccaaaggagagctagaagttgaagagaaagaactggaggtgttgcagaacatcgctgacctgacggtgaaggccgaggaccaggccatgttcaagtgtgaggtgtctgatgagaaggtaacagggaaatggctcaaagatggcgtggaggtgctgcccagcagtcgcatcaagctgacccacatcggaagaatccatcggcttaccatagacgacgtcaagcccgaggacgctggagactatacctttatcccagacggatatgccctctcactgtccgccaaactcaacttcctggaaattaagatcgactatgtcccccgccaagagccacccaagatccatctagacaccaccggcaacatggtttcccagaacaccatcattgtggtggctggcaacaagctgcgcctggacgtggagatctctggagagcctcctcccactgtcgtctgggcaaagggagacacggcaatcacagctgtggaggggcgtgtgaggactgagagccggaaggacctgagctgcttcgtcatagagggggcagagagagaggatgagggcaactacaccatcattgtcaccaaccctgccggagaggacaaggctcatctgtttgtcaagattgttgatgtgcctgactgccctgagaatgtaaagtgtacctcggtgggagaagactgtgccaccatggtctgggacccacccaagttcgacggaggcgcaccagtcacaggctatctcatggagaggaagaagaagggctccaccaggtggacgaagctcaactttgacgtgtacgagggggtgacgtacgaagccaagaggatgattgagggcgtgctctacgagatgagagtgtacgccgtcaatggcattggcatgtcccagcccagcctcaattccaaacccttcatgcccatcgctgcaaccagtgagcctctgggcctcaaggtgcatgatgtcacagacaccacatgtaccctgaagtggctggcccctgagaagatcggtgccggaggcctggacggatacgttattgagtactgcaaggagggagacacggagtgggtggtggcgaacactgagctggtggagagacagaattacacagtccgcaacctccccaccgcggagaagatcaacttcagagtggtggccgtgaacattgctggacgcagcccccctaccggcctgagccagcccgtcactatccgcgagatcgtggaactgcccaagatccgcctccctcgctacttgagacagaagtacattagaagagtgggcgacaaaatcaacctgaccatccccttccagggtaagccacgtcccaaagtgcaatggtacaaggatggggaagagcttgacactcgagttgccagcatacgcaattccgaagtggactctatcctgttcatccgctcggctgagagatcccactctgggaagtacgagttggtcctgcagattgagaacatggaggccagggctattctggaaatcaggatcgtagaggcacctgggcctcccgaggtagtgaaggtcacagacgtttggggcttcaacgctgcgctggagtggaagccaccgaaggacgacggcaactgcgagatcaccggttacacaatacagaaggcggacaagaagacaaatgaatggttcactatctacgagcacaacaggaggacaaactgcacagcctcagacctgatcatgggaaatgagtacatgttccgtgtcttcagcgagaacctcgttggaaagagtgaggatttctgcctcagcaaggacacagccatcatacctaagataggattggagtacaacccacctccattcaaggagaaggatatgcaaagcgcccccaagttcatacagcccctgcttgacaggtctgtggtggcaggctacagtaccaccatcagctctgctgtcaagacttgtcctaaacctaagatcaggtggttgaggaataagattcccctggacgagaaccctaggttcctgatgcagaacaaccagggggttttgaccctgaacatccgcaagcccagtcagtatgacgggggcaagttcacctgcaaggctatcaactctttgggggaggacgtcgtggagtgcaccctactcgtacgagctctcaaggacaaggaagatggagacgagaaa

SEQ ID NO: 121 - protein, 1133 aa, synthetic construct
  REGION 1..1133: note = myosin-binding protein C, fast-type-like
MPEAADAVKPEGEEGEAPAEAEENTAEDDEPLPEGEEGAHQSQEITGLFLQHPETTVAITGTDIVFTAKVDSTTLSRKPTIKWLKGKWLDLGSKAGKHMQFKETFDRATKIYTWDMKIIKVVPGDAGAYRCEVTSKDKCDSSAFDISVEAVQIEEEHDSLAAFKRTDAGEDEGSLDFSALLKATKKKKKPVVDEEKADVWEILKNAQPSEYEKIAFEYGITDLRGLLKRLKKMKTVEPKHSDAFLKRMESAYSVDKGKKIVLQVEVVDPNAQVKWLKNGQEIKSSAKYIIESVGNIRTLTINKCSLADDAAYECVIGEEKSFTEVFVKEPPVTITKLLDDYHVVVGERVEFEVEVSVEGAHVNWMFEDQELSRDTHKYRFKKDGLKHMLIIQEATLDDIGMYWCFTNGGRTKGELEVEEKELEVLQNIADLTVKAEDQAMFKCEVSDEKVTGKWLKDGVEVLPSSRIKLTHIGRIHRLTIDDVKPEDAGDYTFIPDGYALSLSAKLNFLEIKIDYVPRQEPPKIHLDTTGNMVSQNTIIVVAGNKLRLDVEISGEPPPTVVWAKGDTAITAVEGRVRTESRKDLSCFVIEGAEREDEGNYTIIVTNPAGEDKAHLFVKIVDVPDCPENVKCTSVGEDCATMVWDPPKFDGGAPVTGYLMERKKKGSTRWTKLNFDVYEGVTYEAKRMIEGVLYEMRVYAVNGIGMSQPSLNSKPFMPIAATSEPLGLKVHDVTDTTCTLKWLAPEKIGAGGLDGYVIEYCKEGDTEWVVANTELVERQNYTVRNLPTAEKINFRVVAVNIAGRSPPTGLSQPVTIREIVELPKIRLPRYLRQKYIRRVGDKINLTIPFQGKPRPKVQWYKDGEELDTRVASIRNSEVDSILFIRSAERSHSGKYELVLQIENMEARAILEIRIVEAPGPPEVVKVTDVWGFNAALEWKPPKDDGNCEITGYTIQKADKKTNEWFTIYEHNRRTNCTASDLIMGNEYMFRVFSENLVGKSEDFCLSKDTAIIPKIGLEYNPPPFKEKDMQSAPKFIQPLLDRSVVAGYSTTISSAVKTCPKPKIRWLRNKIPLDENPRFLMQNNQGVLTLNIRKPSQYDGGKFTCKAINSLGEDVVECTLLVRALKDKEDGDEK

SEQ ID NO: 122 - protein, 8 aa, synthetic construct
  REGION 1..8: note = myosin-binding protein C, fast-type-like fragment
ADVWEILK

SEQ ID NO: 123 - protein, 14 aa, synthetic construct
  REGION 1..14: note = myosin-binding protein C, fast-type-like fragment
ELEVLQNIADLTVK

SEQ ID NO: 124 - protein, 8 aa, synthetic construct
  REGION 1..8: note = myosin-binding protein C, fast-type-like fragment
EPPVTITK

SEQ ID NO: 125 - protein, 8 aa, synthetic construct
  REGION 1..8: note = myosin-binding protein C, fast-type-like fragment
GELEVEEK

SEQ ID NO: 126 - protein, 7 aa, synthetic construct
  REGION 1..7: note = myosin-binding protein C, fast-type-like fragment
HSDAFLK

SEQ ID NO: 127 - protein, 15 aa, synthetic construct
  REGION 1..15: note = myosin-binding protein C, fast-type-like fragment
IAFEYGITDLRGLLK

SEQ ID NO: 128 - protein, 11 aa, synthetic construct
  REGION 1..11: note = myosin-binding protein C, fast-type-like fragment
IGLEYNPPPFK

SEQ ID NO: 129 - protein, 14 aa, synthetic construct
  REGION 1..14: note = myosin-binding protein C, fast-type-like fragment
INLTIPFQGKPRPK

SEQ ID NO: 130 - protein, 15 aa, synthetic construct
  REGION 1..15: note = myosin-binding protein C, fast-type-like fragment
IVLQVEVVDPNAQVK

SEQ ID NO: 131 - protein, 14 aa, synthetic construct
  REGION 1..14: note = myosin-binding protein C, fast-type-like fragment
LNFDVYEGVTYEAK

SEQ ID NO: 132 - protein, 7 aa, synthetic construct
  REGION 1..7: note = myosin-binding protein C, fast-type-like fragment
LNFLEIK

SEQ ID NO: 133 - protein, 9 aa, synthetic construct
  REGION 1..9: note = myosin-binding protein C, fast-type-like fragment
NAQPSEYEK

SEQ ID NO: 134 - protein, 18 aa, synthetic construct
  REGION 1..18: note = myosin-binding protein C, fast-type-like fragment
RTDAGEDEGSLDFSALLK

SEQ ID NO: 135 - protein, 8 aa, synthetic construct
  REGION 1..8: note = myosin-binding protein C, fast-type-like fragment
SFTEVFVK

SEQ ID NO: 136 - protein, 13 aa, synthetic construct
  REGION 1..13: note = myosin-binding protein C, fast-type-like fragment
VDSTTLSRKPTIK

SEQ ID NO: 137 - DNA, 1551 bp, synthetic construct
  misc_feature 1..1551: note = ATP synthase subunit beta, mitochondrial
  source 1..1551: mol_type = other DNA
atgttaggagctgtgggacgctgctgcaccggggctctccaggcacttaagcctggggtccagcccctgaaggcccttgtaggatcgccatctgtccttggacgcagagactactctgcacctgctgctgctgccgccttcgcccatggcaggatcgtagcggtcatcggtgccgtcgtcgacgtccagttcgatgagggcctcccacccatcctcaatgccctggaagttgctgggcgtgagtccaggctagttctggaggtggcacagcatcttggtgagaacactgtgcgtaccattgccatggatggtactgaaggtcttgtccgtggacagaaggttgtggacactggcgatcccatcagaatcccagtgggtcccgagaccctaggcagaatcatgaatgtcattggagagcccattgatgagagggggccaatctccaccaagcagactgccgccatccacgctgaggccccagagttcactgacatgagtgtggagcaggagatcctggtaactggcatcaaggtggtagatctgctggctccctatgccaagggaggcaaaatcggtctgttcggtggtgctggtgtgggcaagactgtgttgatcatggagctgatcaacaatgtggccaaggcccatggtggttactctgtgtttgccggagtgggagagcgtacccgcgagggaaatgacttgtaccacgagatgattgagtcgggtgtcatcaacctgaaggatgacacctccaaggtggcgctggtgtacggacaaatgaacgagcccccaggcgcccgtgcccgtgtggctctgactggtctgaccgtggcagagtatttccgtgaccaggagggtcaggatgtgctgctcttcatcgataacatcttccgtttcacccaggctggctccgaggtgtctgccctgctgggtcgtatcccctccgctgtgggttaccagcccaccctggccactgacatgggtaccatgcaggagagaatcaccaccaccaagaagggttccatcacctctgtgcaggccatctatgtgccggctgacgatttgactgatcccgcccctgccaccaccttcgctcacttggacgccaccactgtgctgtcccgtgcaatcgctgagctgggtatctaccctgctgtggatcccctggattccacctcccgtatcatggaccccaacattgtcggcgctgagcactatgatgttgctcgtggcgtgcagaagatcctccaggactacaagtccctgcaggatatcattgccatcctgggtatggatgagttgtctgaggaggacaagctgattgtctctcgcgcccgcaagatccagcgtttcctgtcccagcccttccaggtggcggaggtattcaccggtcatgctggcaagctggtgccactcaaggagaccatcagtggcttccagagcattctaaatggtgagtacgatgccctgcccgagcaggctttctacatggttggacccatcgaggaggtggttgagaaggctgcacagatggccaaggatctcgca

SEQ ID NO: 138 - protein, 517 aa, synthetic construct
  REGION 1..517: note = ATP synthase subunit beta, mitochondrial
MLGAVGRCCTGALQALKPGVQPLKALVGSPSVLGRRDYSAPAAAAAFAHGRIVAVIGAVVDVQFDEGLPPILNALEVAGRESRLVLEVAQHLGENTVRTIAMDGTEGLVRGQKVVDTGDPIRIPVGPETLGRIMNVIGEPIDERGPISTKQTAAIHAEAPEFTDMSVEQEILVTGIKVVDLLAPYAKGGKIGLFGGAGVGKTVLIMELINNVAKAHGGYSVFAGVGERTREGNDLYHEMIESGVINLKDDTSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGAEHYDVARGVQKILQDYKSLQDIIAILGMDELSEEDKLIVSRARKIQRFLSQPFQVAEVFTGHAGKLVPLKETISGFQSILNGEYDALPEQAFYMVGPIEEVVEKAAQMAKDLA

SEQ ID NO: 139 - protein, 11 aa, synthetic construct
  REGION 1..11: note = ATP synthase subunit beta, mitochondrial fragment
IGLFGGAGVGK

SEQ ID NO: 140 - protein, 21 aa, synthetic construct
  REGION 1..21: note = ATP synthase subunit beta, mitochondrial fragment
IQRFLSQPFQVAEVFTGHAGK

SEQ ID NO: 141 - protein, 13 aa, synthetic construct
  REGION 1..13: note = ATP synthase subunit beta, mitochondrial fragment
TVLIMELINNVAK

SEQ ID NO: 142 - protein, 10 aa, synthetic construct
  REGION 1..10: note = ATP synthase subunit beta, mitochondrial fragment
VVDLLAPYAK

SEQ ID NO: 143 - DNA, 996 bp, synthetic construct
  misc_feature 1..996: note = L-lactate dehydrogenase A chain-like
  source 1..996: mol_type = other DNA
atgactaccaaggagaagctgatcacccatgtgttggctggtgagcctgttggctcccggagcaaggtgacagttgttggcgtcggcatggttggcatggcctccgcagtcagcgtcctgctcaaggacctgtgcgatgagctgtgcctgattgacgtgatggaagataaactgaagggtgaggtcatggacctgcagcatggcagcctcttctgcaagactcacaagattgtgggcgacaaggactacagtacgactgcccactccaaggtggtggtggtcacagccggtgctcgtcagcaagagggtgagagccgtctgaacctggtgcagcgtaacgtcaacatcttcaaattcataattccccagatcgtcaagtacagccccaacgccatcctgctggtcgtctccaatcctgttgacatcctaacctacgtggcttggaagctgagtggtttcccccgtcaccgcgtcatcggttccggcaccaacctggactctggtcgtttccgccacctgatgggcgagaagctacaccttcacccatccagctgtcacggctggatcattggagaacacggagactccagcgtgcccgtatggagcggtgtgaatgttgccggtgtttccctgaagggcctgaacccagacatgggcacagacgcagacaaggaggactggaagcacgtccacaagatggtggtcgacggtgcctatgaggtcatcaagctgaagggttacacctcctgggctatcggcatgtccgtcgctgacctggttgagagcatcctgaagaacctccacaaagtccaccctgtgtccaccctggtcaagggaatgcacggtgtgaaggaggaggtgtttctcagcgtgccctgcgtgctgggaaacagcggtctgaccgacgtcatccacatgactctgaagcccgaggaggagaagcagctgagcaacagtgccgagaccctatggggcgtacagaaagagctcaccttg

SEQ ID NO: 144 - protein, 332 aa, synthetic construct
  REGION 1..332: note = L-lactate dehydrogenase A chain-like
MTTKEKLITHVLAGEPVGSRSKVTVVGVGMVGMASAVSVLLKDLCDELCLIDVMEDKLKGEVMDLQHGSLFCKTHKIVGDKDYSTTAHSKVVVVTAGARQQEGESRLNLVQRNVNIFKFIIPQIVKYSPNAILLVVSNPVDILTYVAWKLSGFPRHRVIGSGTNLDSGRFRHLMGEKLHLHPSSCHGWIIGEHGDSSVPVWSGVNVAGVSLKGLNPDMGTDADKEDWKHVHKMVVDGAYEVIKLKGYTSWAIGMSVADLVESILKNLHKVHPVSTLVKGMHGVKEEVFLSVPCVLGNSGLTDVIHMTLKPEEEKQLSNSAETLWGVQKELTL

SEQ ID NO: 145 - protein, 8 aa, synthetic construct
  REGION 1..8: note = L-lactate dehydrogenase A chain-like fragment
FIIPQIVK

SEQ ID NO: 146 - protein, 16 aa, synthetic construct
  REGION 1..16: note = L-lactate dehydrogenase A chain-like fragment
LITHVLAGEPVGSRSK

SEQ ID NO: 147 - protein, 14 aa, synthetic construct
  REGION 1..14: note = L-lactate dehydrogenase A chain-like fragment
THVLAGEPVGSRSK

SEQ ID NO: 148 - protein, 9 aa, synthetic construct
  REGION 1..9: note = L-lactate dehydrogenase A chain-like fragment
VHPVSTLVK

SEQ ID NO: 149 - protein, 28 aa, synthetic construct
  REGION 1..28: note = L-lactate dehydrogenase A chain-like fragment
VVVVTAGARQQEGESRLNLVQRNVNIFK